(19) United States
(12) Patent Application Publication
(10) Pub. No.: US 2001/0051184 A1
(43) Pub. Date: Dec. 13, 2001

(54) METHOD FOR USING SOLUBLE CURCUMIN TO INHIBIT PHOSPHORYLASE KINASE IN INFLAMMATORY DISEASES

(76) Inventor: MADALENE C.Y. HENG, NORTHRIDGE, CA (US)

Correspondence Address: ATTN: DAVID A. FARAH. M.D., SHELDON & MAK, 225 SOUTH LAKE AVENUE, SUITE 900, PASADENA, CA, 91101, US

(21) Appl. No.: 09/315,856

(22) Filed: May 20, 1999

(51) Int. Cl. A61K 9/00; A61K 9/62; A61K 9/26; A61K 9/16; A61K 9/50; A61K 33/26; A61K 33/14; A61K 33/00; A61K 35/78

(52) U.S. Cl. 424/461; 424/469; 424/678; 424/724; 424/492; 424/646; 424/717; 424/728

(57) ABSTRACT

The compound curcumin, derived from turmeric, inhibits phosphorylase kinase and, by doing so, exhibits a number of physiological effects related to the control of inflammation and cellular proliferation. However, curcumin is effective only when in solution. Curcumin is almost completely insoluble in water or in oils, but is soluble in alcohols. Accordingly, a method for treating inflammation in a mammal comprising administering curcumin in a solution containing at least one alcohol to a mammal to detectably inhibit the activity of phosphorylase kinase in the blood of the mammal or in a tissue of the mammal. The alcohol is preferably ethanol, 1-propanol, or 2-propanol; most preferably, it is ethanol. Instead of curcumin, a curcumin derivative or curcuminoid can be administered. The method can further comprise the administration of at least one additional compound that can be (1) vitamin D₃ and vitamin D₃ analogues; (2) vitamin A, vitamin A derivatives, and vitamin A analogues (3) a calmodulin inhibitor; (4) an anti-inflammatory drug; (5) a calcium channel blocker; (6) a H1 or H2 histamine blocker; (7) an antioxidant; (8) a polyphenolic compound; (9) a monoterpene; (10) genistein; (11) a soybean derived lectin; and (12) dehydrozingerone. Another aspect of the present invention is a pharmaceutical composition comprising curcumin, a curcuminoid, or a curcumin derivative in a solution containing at least one alcohol, at least one additional compound as described above, and a pharmaceutically acceptable carrier.

The subject matter of this application is related to the following previous applications by Madalene C. Y. Heng (1) application Ser. No. 09/134,604, filed Aug. 14, 1998, and entitled “Therapy for Psoriasis Based on Modulation of Phosphorylase Kinase Activity”; and (2) application Ser. No. 08/518,991, filed Aug. 24, 1995, and entitled “Method for Treating Psoriasis Using Selected Phosphorylase Kinase Inhibitor and Additional Compounds.” These two applications are incorporated herein in their entirety by reference.

The inventor of this invention is an employee of the Veterans Administration Medical Center in Sepulveda, Calif. The United States government, through the Veterans Administration, may have certain rights in this invention.

### BACKGROUND OF THE INVENTION

This invention is directed to the use of curcumin, curcumin derivatives, or curcuminoids in soluble form to inhibit phosphorylase kinase in inflammatory diseases, thus blocking or inhibiting inflammation and its consequences.

Inflammation is mediated by a number of proinflammatory molecules (inflammatory mediators and cytokines) secreted by activated inflammatory cells (neutrophils, T lymphocytes, and macrophages). The secretion of these proinflammatory molecules is an energetic process requiring a considerable amount of energy in the form of adenosine triphosphate (ATP). Glycogen is the main source of energy used by all cells. A key regulator of glycogen metabolism is phosphorylase kinase. When activated, phosphorylase kinase promotes the breakdown of glycogen (glycogenolysis) by phosphorylating (activating) phosphorylase, i.e. by converting non-active phosphorylase b to active phosphorylase a (Carlson G M, Bechtel P J, Graves D J. Chemical and regulatory properties of phosphorylase kinase and cyclic AMP-dependent protein kinase. Advances in Enzymology and Related Areas of Molecular Biology 1980;50:41-115; Malencik D A, Fischer E H (1982). Structure, function and regulation of phosphorylase kinase. In: Calcium and Cell Function, Vol III, pp 161-188). The result of phosphorylase kinase mediated glycogenolysis leads to accumulation of ATP within the cell, thus providing the necessary source of energy for inflammatory reactions.

Phosphorylase kinase is composed of four non-identical subunits (α,β,γ, and δ) tightly bound in a complex of molecular weight 1.2 million daltons (Carlson et al, 1980; Malencik et al. 1982). The enzyme is activated when calcium ions binds to the δ subunit, and deactivated by phosphorylation of the α subunit, a reaction catalyzed by cyclic AMP-dependent protein kinase (type II). Phosphorylase kinase is best studied in skeletal muscle (Salgiver W J, Lawrence J C Jr. Rat skeletal muscle phosphorylase kinase: turnover and control of isozyme levels in culture. American Journal of Physiology 1986;250:(Cell Physiology 19):C365-373), which requires large amounts of ATP for its function. However, the activity of the enzyme has been found to be increased in many, if not all, active cells (Davidson J J, Ozcelik T, Hamacher C, Willems P J, Francke U, Kilimann M W. cDNA cloning of a liver isoform of the phosphorylase kinase α subunit and mapping of the gene to Xp22.2-p22.1, the region of human X-linked liver glycogenosis. Proceedings of the National Academy of Sciences (USA) 1992;89:2096-2100; Heng M C Y, Song M K, Heng M K. Elevated phosphorylase kinase activity in psoriatic epidermis: correlation with increased phosphorylation and psoriatic activity. Br J Dermatol 1994;130:298-306). Furthermore, the activity of the enzyme is crucial for cellular activity and function.

The human genes for the α and β subunits of phosphorylase kinase have been mapped to their chromosomal locations (U. Francke et al., “Assignment of Human Genes for Phosphorylase Kinase Subunits α (PHKA) to Xq12-q13 and β (PHKB) to 16q12-q13,” Am. J. Hum. Genet. 45: 276-282 (1989)).

In inflammatory diseases, including those induced by hypersensitive/allergic, injurious or infectious stimuli, the magnitude and activity of the inflammatory cell population are important with regard to the quantity of inflammatory mediators secreted, as well as the effects and longevity of the inflammatory response. The molecules generated by the inflammatory response may be both stimulatory and destructive. Examples of destructive molecules include destructive free oxygen radicals, peroxynitrites, and lytic enzymes which lyse and digest tissue. Many of these lytic enzymes are contained within cellular lysosomes. These lysosomal hydrolases include β-glucuronidase, β-N-acetylglucosaminidase, cathepsin B, cathepsin D, and acid phosphatase. Curcumin, by inhibiting the activity of phosphorylase kinase in the inflammatory cell, has also been shown to secondarily inhibit the activity of these lysosomal hydrolases (Nirmala C, Puvanakrishnan R. Effect of curcumin on certain lysosomal hydrolases in isoproterenol-induced myocardial infarction in rats. Biochemical Pharmacology 1996; 51:47-51). Similarly, curcumin inhibits synthesis of cytokines, such as tumor necrosis factor and interleukin-1 (Chan M M. Inhibition of tumor necrosis factor by curcumin: a phytochemical. Biochemical Pharmacology 1995;49:1551-1556). In addition, curcumin has inhibitory effects on cytokine-induced generation of peroxynitrites (Chan M M, Ho X T, Huang H I. Effects of three dietary phytochemicals from tea, rosemary and tumeric on inflammation-induced nitrite production. Cancer Letters 1995;96:23-29), and free radical formation by inflammatory macrophages (Joe B, Lokesh B R. Role of capsaicin, curcumin and dietary n-2 fatty acids in lowering the generation of reactive oxygen species in rat peritoneal macrophages. Biochimica et Biophysica Acta 1994;1224:255-263; Osawa T, Sugiyama Y, Inayoshi M, Kawakishi S. Antioxidative activity of tetrahydrocurcuminoids. Bioscience, Biotechnology and Biochemistry 1995;59:1609-1912). These inhibitory effects observed with curcumin are secondary to upstream suppression of energy supply to the inflammatory cells through phosphorylase kinase inhibition.

An example of the stimulatory effects of the inflammatory response is observed in the role of inflammatory cells in atherosclerosis. Oxygen reactive species (oxygen free radicals) released by inflammatory cells (neutrophils, T lymphocytes and activated macrophages) damage tissues, resulting in secretion of growth factors and stimulatory cytokines, with excessive stimulation of vascular smooth muscle cell proliferation and eventual aggravation of arterial stenosis and atherosclerosis (Ross R. The pathogenesis of atherosclerosis: a perspective for the 1990s. Nature 1993;362:801-809; Jonasson L, Holm J, Skalli O, Bondjers G, Hansson G K. Regional accumulations of T cells, macrophages and smooth muscle cells in the human atherosclerotic plaque. Arteriosclerosis 1986;6:131-138; Heng M K, Heng M C Y. Heat-shock protein 65 and activated (γ/δ T-cells in injured arteries. Lancet 1994;344:921-923). By suppression of phosphorylase kinase activity, and therefore inflammatory cell activity, curcumin may be beneficial in inhibiting the atherosclerotic process. In support of this premise are studies showing that curcumin is able to prevent ischemialinjury-induced changes in the heart (Dikshit M, Rastogi L, Shukla R, Srimal R C. Prevention of ischemia-induced biochemical changes by curcumin and quinidine in the cat heart. Indian Journal of Medical Research 1995; 101:31-35), and to inhibit vascular smooth muscle cell proliferation (Huang H C, Jan T R, Yeh S F. Inhibitory effect of curcumin, an anti-inflammatory agent, on vascular smooth muscle cell proliferation. European Journal of Pharmacology 1992;221:381-384). In rats, curcumin protects against isoproterenol-induced myocardial infarction (Nirmala C, Puvanakrishnan R. Protective role of curcumin against isoproterenol-induced myocardial infarction in rats. Molecular and Cellular Biochemistry 1996; 159:85-93). Thus, curcumin has the potential of being a potent anti-atherosclerotic drug.

In the field of gingivitis and inflammatory bowel disease, bacterial antigens play an important role in initiating the inflammatory response (Watanabe A, Takeshita A, Kitano S, Hanazawa S. The CD14-mediated signal pathway of Porphyromonas gingivalis lipopolysaccharide in human gingival fibroblasts. Infection and Immunity 1996;64:4488-4494; Leung F, Heng M C Y, Allen S, Seno K, Lam K, Leung J W C, Heng M K. Immunological events in dextran sulfate sodium-induced colitis in rats: a morphological, immunocytochemical and ultrastructural study. Accepted for presentation at the Meeting of the Gastroenterological Association, New Orleans, May 1998). It has been shown in these diseases that lipopolysaccharides (LPS), a constituent of the cell membrane of Gram negative bacteria, activate certain genes responsible for the inflammatory response, and that curcumin decreases LPS-induced expression of such genes (Watanabe et al., 1996).

In tumor cells, inflammatory cytokines, such as tumor necrosis factor, have been shown to activate transcription factor (growth-promoting) genes such as NF-κβ. Curcumin has been shown to inhibit the activation of NF-κβ in tumor-necrosis factor-activated malignant cells (Singh S, Aggarwal B B. Activation of transcription factor NF-κβ is suppressed by curcumin (diferuloylmethane). J Biol Chem 1995;270:24995-5000). Curcumin has an antiproliferative effect against human breast tumor cell lines (Mehta K, Pantazis P, McQueen T, Aggawal B B. Antiproliferative effect of curcumin (diferuloylmethane) against breast tumor cell lines. Anti-Cancer Drugs 1997;8:470-481; Verma S P, Salamone E, Goldin B. Curcumin and genistein, plant natural products, show synergistic inhibitory effects on the growth of human breast cancer MCF-2 cells induced by estrogenic pesticides. Biochemical and Biophysics Research Communications 1997;233 :692-696). In chemoprevention, curcumin has been shown to decrease tumor yield in oral and colon cancer (Azuine M A, Bhide S V. Adjuvant chemoprevention of experimental cancer: cachetin and dietary tumeric in forestomach and oral cancer models. Journal of Ethnopharmacology 1994;44:211-217; Rao C V, Rivenson A, Simi B, Reddy B S. Chemoprevention of colon cancer by dietary curcumin. Annals of the New York of Sciences 1995;768:201-204). In addition, curcumin also inhibits the formation of benzopyrene-derived DNA-adducts (Deshpande S S, Maru G B. Effects of curcumin on the formation of benzo[a]pyrene DNA adducts in vitro. Cancer Letters 1995;96:71-80). Both natural and synthetic curcuminoids have been observed to show antimutagenic and anticarcinogenic activity (Anto R J, George J, Babu K V, Rajasekharan K N, Kuttan R. Antimutagenic and anticarcinogenic activity of natural and synthetic curcuminoids. Mutation Research 1996;370:127-131).

In the field of ophthalmology, curcumin has been shown to protect against cataract formation (Awasthi S, Srivatava S K, Piper I T, Chaubey M, Awasthi Y C. Curcumin protects against 4-hydroxy-2-nonenal-induced cataract formation in rat lenses. American Journal of Clinical Nutrition 1996;64:761-766).

In radiation oncology, curcumin protects against radiation-induced toxicity (Thresiamma K C, George J, Kuttan R. Protective effect of curcumin, ellagic acid and bixin on radiation induced toxicity. Indian Journal of Experimental Biology 1996;34:845-847), bleomycin-induced lung injury (Venkatesan N, Punithavathi V, Chandrakasan G. Curcumin protects bleomycin-induced lung injury in rats. Life Sciences 1997;61 :PL51-58), and cyclophosphamide-induced lung injury (Venkatesan N, Chandrakasan G. Modulation of cyclophosphamide-induced early lung injury by curcumin, an anti-inflammatory antioxidant. Molecular and Cellular Biochemistry. 1995;142:79-87).

In rheumatology, curcumin lowers the levels of an acidic glycoprotein (GP A72) in arthritic rats, with concomitant lowering of paw inflammation (Joe B, Rao U J, Lokesh B R. Presence of an acidic glycoprotein in the serum of arthritic rats: modulation by capsaicin and curcumin. Molecular and Cellular Biochemistry 1997;169:125-134).

In patients with infectious disease, curcumin has been shown to inhibit proteases secreted by human immunodeficiency viruses, HIV-1 and HIV-2 (Sui Z, Salto R, Li J, Craik C, Otiz de Montellano P R. Inhibition of HIV-1 and HIV-2 proteases by curcumin and curcumin-boron complexes. Bioorganic and Medicinal Chemistry 1993;1:415-422). Cucumin has also been shown to have nematocidal properties (Kiuchi F, Goto Y, Sugimoto N, Akao N, Kondo K, Tsuda Y. Nematocidal activity of tumeric: synergistic action of curcuminoids. Chemical and Pharmaceutical Bulletin 1993;41:1640-1643).

Despite these properties of curcumin, there is a need for improved compositions and methods for the delivery of curcumin to subjects to be treated. Whether curcumin is in soluble form or not is of critical importance because its anti-phosphorylase kinase activity and its anti-inflammatory effect depends on the presence of curcumin in a dissolved state. This concept is supported by studies showing the lack of inflammatory effect of curcumin when given together with oil products (Reddy A C, Lokesh B R. Studies on anti-inflammatory activity of spice principles and dietary n-3 polyunsaturated fatty acids on carrageenan-induced inflammation in rats. Annals of Nutrition and Metabolism 1994;38:349-358), and its efficacy when given together with an alcohol-based substance such as eugenol (Reddy and Lokesh 1994). Further support is provided by the lack of anti-fungal activity by curcumin in oil (Apisariyakul A, Vanittanakom N, Buddhasukh D. Antifungal activity of tumeric oil extracted from Curcuma longa (Zingiberaceae). Journal of Ethnopharmacology 1995;49:163-169).

Therefore, there is a need for improved compositions and methods for the administration of curcumin to allow the curcumin to remain in solution. There is further a need for improved compositions and methods for the administration of curcumin, curcumin derivatives, and curcuminoids, alone or together with additional compounds, that allow the curcumin, curcumin derivatives, or curcuminoids to remain in solution and do not affect the activity of the additional compounds if present.

### SUMMARY

I have discovered that curcumin is soluble in a solution or a gel that contains an alcohol and that the administration of curcumin in solution greatly improves the activity of curcumin in inhibiting phosphorylase kinase and exerting anti-inflammatory and other physiological effects.

One aspect of the present invention is a method for treating inflammation in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal. The mammal that can be treated can be a human or a socially or economically important animal such as a cow, a horse, a sheep, a goat, a pig, a dog, or a cat.

Typically, the at least one alcohol is selected from the group consisting of alcohols with from 1 to 6 carbon atoms. Preferably, the at least one alcohol is selected from the group consisting of alcohols with from 1 to 3 carbon atoms. Typically, the at least one alcohol is saturated and is monohydric. More preferably, the at least one alcohol is selected from the group consisting of ethanol, 1-propanol, and 2-propanol. Most preferably, the alcohol is ethanol.

In this method, one of the following stages of inflammation can be inhibited by the administration of soluble curcumin: (1) the migration of γ/δ T cells occurring at about 30 minutes to about 4 hours after the inflammatory stress; (2) the migration of neutrophils beginning at about 18-24 hours after the inflammatory stress;(3) the migration of macrophages beginning at about 24 hours after the inflammatory stress; and (4) the migration of α/β T cells and other cells such as eosinophils beginning at about 48 hours to 72 hours after the inflammatory stress.

The curcumin can be administered as a boron complex, or in a liposome.

The boron complex can be one of

(1) a difluoroboron complex;

(2) a mixed complex in which the two fluorine atoms of a difluoroboron complex are replaced with the carboxyl oxygens of oxalic acid;

(3) a mixed complex in which the two fluorine atoms of a difluoroboron complex are replaced with a carboxyl group and a hydroxyl group of citric acid;

(4) a mixed complex in which the two fluorine atoms of a difluoroboron complex are replaced with the two hydroxyl groups of dibenzyl tartramide; and

(5) a mixed complex in which the two fluorine atoms of a difluoroboron complex are replaced with a second molecule of curcumin.

If the curcumin is administered in a liposome, the curcumin can be administered in a preparation selected from the group consisting of a skin preparation, an eye drop preparation, a nasal drop preparation, an oral preparation, a pharyngeal preparation, a rectal preparation, a vaginal preparation, a bladder preparation, a urethral preparation, and a bronchial preparation.

In another alternative, the method can comprise administering a curcuminoid or curcumin derivative instead of or in addition to curcumin. The curcuminoid or curcumin derivative can comprise:

(1) curcumin;

(2) a curcuminoid of formula (I) in which:

(a) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;

(b) R₁ is —H; R₂ is —OH; R₃ is —H or —OH; R₄ is —H, R₅ is —H; R₆ is —OH; and R₇ is —H or —OH;

(c) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;

(d) R₁ is —OH, R₂ is —OH, R₃ is —OH, R₄ is —H or —OH, R₅ is —OH, R₆ is —OH; and R₇ is —OH;

(e) R₁ is —OCH₃; R₂ is —OCH₃; R₃ is —OCH₃; R₄ is —H or —OH; R₅ is —OCH₃; R₆ is —OCH₃; and R₇ is —OCH₃;

(f) R₁ is —H; R₂ is —OCH₃; R₃ is —OCH₃; R₄ is —H or —OH; R₅ is —H; R₆ is —OCH₃; and R₇ is —OCH₃;

(g) R₁ is —H; R₂ is —OH; R₃ is —H; R₄ is —H; R₅ is —H or —OH; R₆ is —OH; and R₇ is —H;

(h) R₁ is —H; R₂ is —OCH₃; R₃ is —H; R₄ is —H; R₅ is —H or —OH; R₆ is —OCH₃; and R₇ is —H; or

(i) R₁ is —OH; R₂ is —OCH₃; R₃ is —H or —OH; R₄ is H or —OH; R₅ is —OH; R₆ is —OCH₃; and R₇ is —H or —OH;

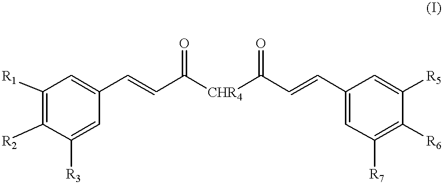

(3) a curcuminoid of formula (II) in which the alternatives for R₁ through R₇ are the same as those recited in paragraph (2);

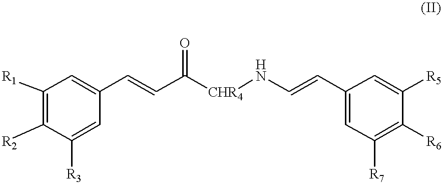

(4) a curcuminoid of formula (III) in which the alternatives for R₁ through R₇ are the same as those recited in paragraph (2);

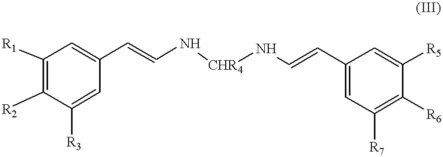

(5) the compound of formula (IV) in which X is —H, the compound being designated furfural curcuminoid;

(6) an analogue of furfural curcuminoid in which X is —OH, ethyl, methyl, or acetyl;

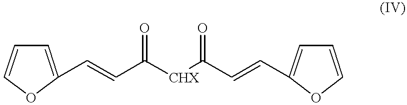

(7) the compound of formula (V) in which X is —H, the compound being designated salicyl curcuminoid;

(8) an analogue of salicyl curcuminoid in which X is —OH, ethyl, methyl, or acetyl;

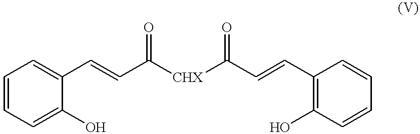

(9) the compound of formula (VI) in which X is —H, the compound being designated veratryl curcuminoid;

(10) an analogue of veratryl curcuminoid in which X is —OH, ethyl, methyl, or acetyl;

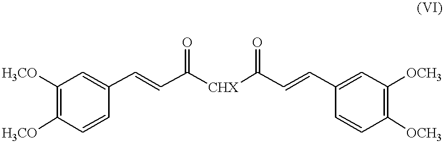

(11) the compound of formula (VII) in which X is —H, the compound being designated p-anisyl curcuminoid;

(12) an analogue of p-anisyl curcuminoid in which X is —OH, ethyl, methyl, or acetyl;

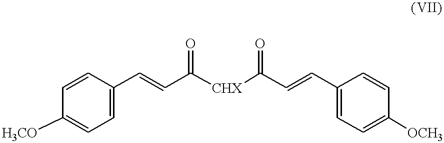

(13 the compound of formula (VIII) in which X is —H, the compound being designated piperonal curcuminoid;

(14) an analogue of piperonal curcuminoid in which X is —OH, ethyl, methyl, or acetyl;

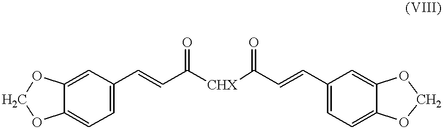

(15) a tetrahydrocurcuminoid of formula (IX) in which the alternatives for R₁ through R₇ are the same as those recited in paragraph (2);

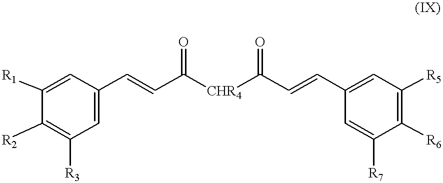

(16) a curcuminoid of formula (X) in which the alternatives for R₁ through R₇ are the same as those recited in paragraph (2);

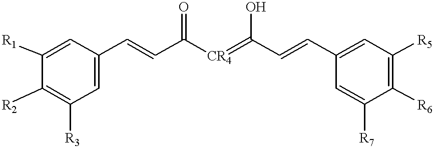

(17) a curcuminoid of formula (XI) in which the alternatives for R₁ through R₇ are the same as those recited in paragraph (2);

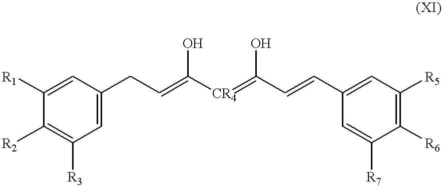

(18) a reduced curcuminoid of formula (XII) in which the alternatives for R₁ through R₇ are the same as those recited in paragraph (2);

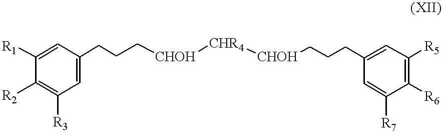

(19) derivatives of the compounds recited in (2) through (18) in which any of the methoxy groups are replaced with lower alkoxy groups selected from the group consisting of ethoxy, n-propoxy, and isopropoxy;

(20) derivatives of the compounds recited in (2) through (18) in which any of the hydroxy groups of the phenolic moieties are substituted with an acyl group selected from the group consisting of acetyl, propionyl, butyryl, and isobutyryl;

(21) analogues of the compounds recited in (2), (3), and (5) through (16) in which one or both of the carbonyl (CO) groups are replaced by amino (NH) groups in analogy with formulas II and III; and

(22) analogues of the compounds recited in (2), (3), and (5) through (16) in which one or both of the oxygens of the carbonyl groups are replaced by sulfur to form thiocarbonyl groups.

Another aspect of the present invention is a method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal. In one alternative, the condition or disease can be selected from the group consisting of psoriasis, skin wounds, burns and scalds, scars, chemical-, radiation-, and sun-induced injury to the skin, smoking-induced injury to the skin, allergic and hypersensitive reactions, hay fever, periodontal disease, gingivitis, eczemas, and skin infections (bacterial, viral, fungal, or mycoplasmal). Alternatively, the condition or disease can be selected from the group consisting of arthritis, systemic lupus erythematosus (SLE), connective tissue diseases, atherosclerosis, Alzheimer's Disease, the inflammatory process that occurs during partial or complete blockage of an artery such as a coronary artery, gastritis, chronic hepatitis, chronic diverticulitis, osteomyelitis, inflammatory bowel diseases, pelvic inflammatory disease, chronic prostatitis, sinusitis, neuritis, neuropathies, and radiation- and smoking-induced injury. In another alternative, the condition or disease can be selected from the group consisting of benign and malignant tumors, including metastatic tumors, of a tissue selected from the group consisting of breast, prostate, lung, skin, melanomas, brain, liver, pancreas, gastric, intestinal, colon, kidney, bladder, cervix, ovary, uterus, central nervous system, sinuses, eye, ear, bone, and thyroid, lymphomas and leukemias. In still another alternative, the condition or disease can be selected from the group consisting of infections caused by bacteria, superficial fungi, deep fungi, viruses, mycoplasmas, and parasites. In yet another alternative, the condition or disease can be diabetes. In another alternative, the condition or disease can be a neurodegenerative condition.

Alternatively, the curcuminoids or curcumin derivatives described above can be used in these methods.

These methods can further comprise administering to the mammal at least one additional compound, the additional compound being selected from the group consisting of:

(1) vitamin D₃ and vitamin D₃ analogues;

(2) vitamin A, vitamin A derivatives, and vitamin A analogues

(3) a calmodulin inhibitor;

(4) an anti-inflammatory drug;

(5) a calcium channel blocker;

(6) a H1 or H2 histamine blocker;

(7) an antioxidant;

(8) a polyphenolic compound;

(9) a monoterpene;

(10) genistein;

(11) a soybean derived lectin; and

(12) dehydrozingerone.

The additional compound can be selected from the group consisting of calcitriol, calcipotriene, calcipotriol, and tacalcitol.

The additional compound can be selected from the group consisting of vitamin A, a vitamin A derivative, and a vitamin A analogue.

The additional compound can be a calmodulin inhibitor selected from the group consisting of zinc, cyclosporin A, anthralin, and trifluoroperazine.

The additional compound can be an anti-inflammatory drug selected from the group consisting of a corticosteroid, a substance P inhibitor, a capsaicin-sensitive vanilloid receptor inhibitor, a cyclo-oxygenase inhibitor, and another non-steroidal anti-inflammatory agent.

The additional compound can be a calcium channel blocker selected from the group consisting of diltiazem, nifedepine, isradipine, and verapamil.

The additional compound can be a H1 histamine blocker or a H2 histamine blocker, wherein the H1 histamine blocker is selected from the group consisting of carbinoxamine maleate, clemastine fumarate, diphenhydramine hydrochloride, dimenhydrinate, pyrilamine maleate, tripelennamine hydrochloride, tripelennamine citrate, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, acrivastine, cetirizine hydrochloride, astemizole, levocabastine hydrochloride, loratadine, and terfenadine, and wherein the H2 histamine blocker is selected from the group consisting of cimetidine, ranitidine, famotidine, and nizatidine.

The additional compound can be the an antioxidant selected from the group consisting of α-tocopherol, β-carotene, superoxide dismutase, catalase, and reduced glutathione.

The additional compound can be a polyphenolic compound selected from the group consisting of (−)epigallocatechin-3-gallate, epigallocatechin, rutin, catechin, epicatechin, naringin, naringenin, and gallotanin.

The additional compound can be a monoterpene selected from the group consisting of d-limonene and perillyl alcohol.

The additional compound can be genistein.

The additional compound can be the soybean derived lectin soybean agglutinin.

The additional compound can be dehydrozingerone.

Another aspect of the present invention is a pharmaceutical composition comprising: pharmaceutical composition comprising:

(1) curcumin, a curcuminoid, or a curcumin derivative in a solution containing at least one alcohol, the curcumin, curcuminoid, or curcumin derivative being present in a quantity sufficient to detectably inhibit the activity of phosphorylase kinase in the blood of the mammal or in a tissue of the mammal to which the composition is administered;

(2) at least one additional compound, the additional compound being selected from the group consisting of:

(a) vitamin D₃ and vitamin D₃ analogues;

(b) vitamin A, vitamin A derivatives, and vitamin A analogues

(c) a calmodulin inhibitor;

(d) an anti-inflammatory drug;

(e) a calcium channel blocker;

(f) a H1 or H2 histamine blocker;

(g) an antioxidant;

(h) a polyphenolic compound;

(i) a monoterpene;

(j) genistein;

(k) a soybean derived lectin; and

(l) dehydrozingerone; and

(3) a pharmaceutically acceptable carrier.

The curcumin, curcumin derivative, or curcuminoid can be present in the form of a boron complex, or in a liposome.

### BRIEF DESCRIPTION OF THE DRAWINGS

These and other features, aspects, and advantages of the present invention will become better understood with reference to the following description, appended claims, and the accompanying drawings where:

FIG. 1 is a diagram of the subunit structure of the enzyme phosphorylase kinase, showing the effects of various drugs on each subunit;

FIG. 2 is a photograph of the skin of a psoriatic patient treated with 1% curcumin in an ointment (oil) base without an alcohol, showing no improvement after 4 weeks of treatment; note the yellow color of the treated skin;

FIG. 3a is a photomicrograph, at ×500 magnification, of an immunohistochemical preparation of rat artery 1 hour post-ligation, showing abundant Hsp60+ protein present both intracellularly (single arrow) and extracellularly (double arrow); the fibrillary nature of the Hsp60+ protein is noted when Hsp60 is secreted extracellularly (double arrows); the protein appears more homogeneous when present intracellularly (single arrows);

FIG. 3b is an immunoelectron microscopic preparation at ×130,000 showing immunogold-labeled Hsp60 (single arrows) co-localizing with a fibrillary tannic acid-staining protein; note immunogold labeling (single arrows) of both the tannic acid stained aggregated protein as well as individual protein strands;

FIG. 4a is a photomicrograph of an immunohistochemical preparation of rat artery 4 hr post-ligation at ×400 showing a TCRγ/δ+ T cell with dendritic processes (single arrow) in the intima;

FIG. 4b is a photomicrograph of an immunohistochemical preparation of rat artery at ×400 24 hr post-ligation showing an activated dendritic γ/δ T cell expressing MHC Class II (RT1b+) molecules (single arrows); note MHC Class II expression of non-dendritic T cells, presumably α/β T cells, in the arterial lumen (double arrows) suggesting that these luminal cells are secondarily cytokine-activated rather than primarily antigen-activated;

FIG. 4c is a photomicrograph at ×400 of an immunohistochemical preparation of rat artery 24 hr post ligation showing activated IL-2R+ dendritic T cells in the intima and upper media (single arrows), providing supporting evidence that these dendritic cells are primarily antigen-activated;

FIG. 5a is an electron micrograph at ×11,500 showing a rat artery 4 hr post ligation showing a dendritic γ/δ T cell; note the presence of the nucleus with dense lymphoid nuclear chromatin (N), the long and thin dendritic process (double arrows) and electron-dense cytoplasmic granules (G) of varying sizes;

FIG. 5b is an inset at ×30,000 magnification of FIG. 5a; the closeness of the surface contact (single arrows) between the fibrillary tannic acid-stained Hsp60 (HSP) and the dendritic γ/δ T cell is better seen at the higher magnification of the inset;

FIG. 6 depicts photomicrographs of immunohistochemical preparations in rat arteries 72 hr post-ligation; (a) ED1+ macrophages exclusively in the adventitia at ×400; (b) sparse TCRα/β T cells (single arrows) scattered among the abundant infiltrate shows by staining of adjacent section (FIG. 6a) to be composed predominantly of macrophages (×100); (c) inset of FIG. 6b showing a magnified view of scattered TCRα/β+ T cells (single arrows) among an abundant infiltrate of predominantly macrophages (×600);

FIG. 7a depicts an electron micrograph at ×7500 of a rat artery 24 hours post-ligation showing a dendritic γ/δ T cell (DT)-macrophage (M) interaction with contact between the plasma membranes of the respective cells (single arrow); note the dendritic morphology of the γ/δ T cell with thin and long dendritic processes (double arrow), cytoplasmic granules (G), and cerebriform nucleus with lymphoid dense-chromatin nuclear pattern; compare these features with that of the macrophage with absence of cytoplasmic granules, a nucleus with thin rim of cytoplasm, and abundant well-developed rough endoplasmic reticulum (R);

FIG. 7b shows an electron micrograph at ×17,000 of a rat artery 72 hours post-ligation showing several smooth muscle cells (SMC) in the neointima, contributing to significant intimal thickening; note the prominent basement membrane (single arrows) characteristic of smooth muscle cells (I=internal elastic lamina);

FIG. 8 shows a light photomicrograph of a rat artery 3 months post-ligation at ×150 showing focal intimal thickening (IT) distal to the site of arterial ligation (single arrow); note the normal intima of the segment proximate to the site of arterial ligation (double arrows);

FIG. 9a shows a photomicrograph at ×200 of an immunohistochemical preparation of pre-curcumin-treated active psoriatic skin; note infiltration of epidermis and dermis by abundant CD3+ T lymphocytes, seen to migrate outside the blood vessels both in the dermis (single arrows) and epidermis (double arrows);

FIG. 9b shows a photomicrograph at ×200 of an immunohistochemical preparation of curcumin-treated resolving psoriatic skin; note lack of T lymphocytes in epidermis; T lymphocytes (CD3+) are present only within dermal blood vessels (single arrows); also note lack of migration of T lymphocytes outside blood vessels in curcumin-treated skin;

FIG. 10 is a graph showing phosphorylase kinase activity in untreated and treated psoriatic epidermis;

FIG. 11 is a graph showing the expression of transferrrin receptors in untreated and treated psoriatic epidermis;

FIG. 12 is a graph showing the existence of parakeratosis in untreated and treated psoriatic epidermis;

FIG. 13 is a graph showing the density of epidermal T cells per high power field (hpf) in untreated and treated psoriatic epidermis;

FIG. 14 shows comparative photomicrographs showing the existence of parakeratosis in the stratum comeum of untreated psoriatic (panel A), curcumin-treated psoriatic (panel B), vitamin D₃-analogue treated (panel C) and normal (panel D) epidermis;

FIG. 15 shows comparative photomicrographs showing the occurrence of CD8+ T cells, detected immunohistochemically, in untreated psoriatic (panel A), curcumin-treated psoriatic (panel B), vitamin D₃-analogue treated (panel C); and untreated psoriatic labeled with CD3 epitope (panel D) epidermis; and

FIG. 16 shows comparative photomicrographs showing the occurrence of cells expressing HLA-DR, detected immunohistochemically, in untreated psoriatic (panel A), vitamin D₃-analogue treated psoriatic (panel B), and curcumin-treated (panel C) epidermis.

### DESCRIPTION

I have developed a new method for treatment of a number of conditions that utilizes the discovery that the activity of the enzyme phosphorylase kinase (PK) is increased in these conditions. In particular, the method uses at least one of the following compounds: (1) the phosphorylase kinase inhibitor curcumin; (2) soluble derivatives of curcumin; (3) soluble curcuminoids; and (4) soluble compounds with similar or related active chemical structure, in soluble form to decrease the activity of phosphorylase kinase.

The conditions that can be treated by this method include, but are not limited to, the following:

(1) dermatological and mucosal inflammatory diseases, such as psoriasis, skin wounds, burns and scalds, scars, chemical-, radiation-, and sun-induced injury to the skin, smoking-induced injury to the skin, allergic and hypersensitive reactions, hay fever, periodontal disease, gingivitis, eczemas, and skin infections (bacterial, viral, fungal, or mycoplasmal);

(2) inflammatory diseases such as arthritis, systemic lupus erythematosus (SLE), connective tissue diseases, atherosclerosis, Alzheimer's Disease, gastritis, chronic hepatitis, chronic diverticulitis, osteomyelitis, inflammatory bowel diseases such as colitis and Crohn's disease, pelvic inflammatory disease, chronic prostatitis, sinusitis, neuritis, neuropathies, and radiation- and smoking-induced injury;

(3) benign and malignant tumors, including metastatic tumors (breast, prostate, lung, skin, melanomas, brain, liver, pancreas, gastric, intestinal, colonic, kidney, bladder, cervix, ovary, uterus, central nervous system, sinuses, eye, ear, bone, and thyroid) or lymphomas and leukemias;

(4) infections, such as infections caused by bacteria, superficial and deep fungi (dermatophytes, sporotrichium, histoplasma, blastomyces), viruses (including herpes simplex virus, varicella zoster virus, adenovirus, and human immunodeficiency virus), mycoplasmas, and parasites (nematodes, other worms, and other pathogenic parasites, such as organisms causing filariasis, schistosomiasis, and malaria);

(5) diabetes;

(6) injury induced by alcohol consumption or administration or consumption of drugs of abuse; and

(7) neurodegenerative conditions.

In the case of the conditions and diseases in category (2), the activity of curcumin may also restore normal activity of lysosomal membranes, thus reducing or blocking the release of hydrolases such as cathepsins that cause tissue damage (C. Nirmala & R. Puvanakrishnan, “Effect of Curcumin on Certain Lysosomal Hydrolases in Isoprotenerol-Induced Myocardial Infarction in Rats,” Biochem. Pharmacol. 51: 47-51 (1996)).

In the case of the conditions and diseases in category (3), the activity of the curcumin or related agent inhibits phosphorylase kinase in the rapidly growing neoplastic cells, thereby slowing down the growth rate of these cells. In the case of the conditions and diseases in category (4), the activity of the curcumin or related agent inhibits phosphorylase kinase in the infectious agent or in cells infected by the infectious agent, in the case of viruses, thereby inhibiting the proliferation of the infectious agent (A. Apisariyakul et al., “Antifungal Activity of Turmeric Oil Extracted from Curcuma longa (Zingiberaceae),” L. Ethnopharmacol. 49: 163-169 (1995)).

Although Applicant does not intend to be bound by this theory, as detailed below, in Example 5, it is believed that the inflammatory process occurs in a number of successive stages. In the first of these stages, the active, migrating cells are predominantly γ/δ T cells. This stage typically occurs at about 30 minutes to about 4 hours after the occurrence of the inflammatory stress. The γ/δ T cells are predominantly responsible for triggering the initiation of the immune inflammatory cascade. The second step involves the activity of neutrophils in secreting leukotrienes. This typically occurs at about 4 to about 24 hours after the inflammatory stress. The third stage involves the migration of neutrophils. This stage typically occurs beginning at about 18-24 hours after the inflammatory stress and continues for some time thereafter. The fourth stage involves the migration of macrophages. This stage typically occurs at about 24 hours after the inflammatory stress. The fifth stage involves the migration of α/β T cells and other cells such as eosinophils. This stage typically occurs beginning at about 48 to 72 hours and continues for some time thereafter.

Phosphorylase kinase inhibitors such as curcumin, curcumin derivatives, and curcuminoids inhibit the migratory activity of inflammatory cells. This includes the first, third, fourth, and fifth stages of inflammation. Accordingly, curcumin, curcumin derivatives, and curcuminoids, alone or together with additional compounds, can block or inhibit any or all of the following stages of inflammation: (1) the migration of γ/δ T cells occurring at about 30 minutes to about 4 hours after the inflammatory stress; (2) the migration of neutrophils beginning at about 18-24 hours after the inflammatory stress; (3) the migration of macrophages at about 24 hours after the inflammatory stress; and (4) the migration of α/β T cells and other cells such as eosinophils beginning at about 48 hours to 72 hours after the inflammatory stress.

Methods according to the present invention can be used to treat both humans and other animals, including economically and socially important animals such as cattle, sheep, horses, goats, pigs, dogs, and cats.

I. Basis of the Activity

The basis of the activity of the curcumin preparations used in the present invention is the inhibition of phosphorylase kinase (PK).

Although this enzyme (PK) is known to be abundant in muscles (E. G. Krebs, “Phosphorylation-Dephosphorylation of Enzymes,” Annu. Rev. Biochem. 48:923-959 (1979); P. Cohen, “The Role of Protein Phosphorylation in Neural and Hormonal Control of Cellular Activity,” Nature 296:613-620 (1982)), it has never previously been reported to be present in human epidermis.

Phosphorylase kinase, also known as ATP-phosphorylase b phosphotransferase (J. J. Davidson et al., “cDNA Cloning of a Liver Isoform of the Phosphorylase Kinase a Subunit and Mapping of the Gene to Xp-22.2-p22.1, the Region of Human X-Linked Liver Glycogenolysis,” Proc. Natl. Acad. Sci. USA 89:2096-2100 (1992)), integrates multiple signal transduction pathways and links them to the degradation of glycogen catalyzed by glycogen phosphorylase, thus generating ATP for subsequent metabolism. Specifically, phosphorylase kinase stimulates glycogenolysis by activating serine moieties in glycogen phosphorylase and by transferring the resulting ATP to convert phosphorylase b to phosphorylase a, which becomes available for phosphorylation-dephosphorylation reactions (J. J. Davidson et al. (1992), supra; P. Cohen (1978), supra; E. G. Krebs (1979), supra; P. Cohen (1982), supra). These ATP-dependent phosphorylation reactions mediated by phosphorylase kinase are: (a) triggered by calcium-calmodulin, because phosphorylase kinase is a calmodulin-containing enzyme; and (b) cAMP-dependent, because its activation status depends on Type I cAMP-dependent protein kinases (J. J. Davidson et al. (1992), supra; P. Cohen (1978), supra; E. G. Krebs (1979), supra; P. Cohen (1982), supra).

In this way, the pathways signaled by injury, including physical injury, infections, and allergic reactions, are linked to ATP-dependent events including increased cell cycling (G. G. Krueger (1981), supra), increased cell division (G. G. Krueger (1981), supra), increased keratinocyte mobility, and increased terminal differentiation (M. C. Y. Heng et al., (1992), supra). These result in psoriasiform hyperproliferation, the clinical manifestation of psoriasis, as well as the response seen in wounds, burns, and eczemas.

Phosphorylase kinase levels are linked to calmodulin levels and to calmodulin/cAMP ratios (D. A. Malencik et al., “Binding of Protein Kinase Substrates by Fluorescently Labeled Calmodulin,” Biochem. Biophys. Res. Commun. 108: 266-272 (1982). These are then related to increased psoriatic activity and to the pathophysiological sequelae of wounds, bums, and eczema, as well as the other pathological conditions recited above.

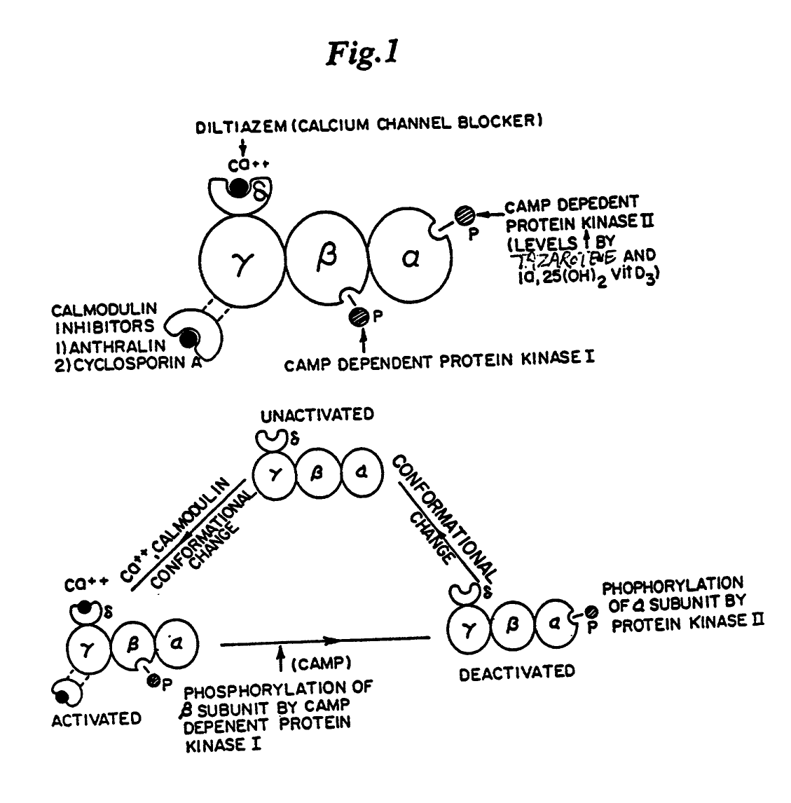

The enzyme phosphorylase kinase (PK) consists of four subunits, with a structure of (αβγδ)₄; the δ subunit is calmodulin. The α and β subunits are the regulatory subunits, with the γ subunit being the catalytic subunit. The enzyme is activated by an influx of calcium ions into the cell from the extracellular fluid, whereupon binding of calcium ions to the calmodulin (δ) subunit results in a conformational change in the molecule, exposing the phosphate binding site on the β subunit to be phosphorylated by cAMP-dependent protein kinase I activating the enzyme. Activated phosphorylase kinase also associates reversibly with another molecule of calmodulin. As cAMP levels rise intracellularly, a second phosphate binding site on the α subunit is phosphorylated by cAMP-dependent protein kinase II, whereupon the molecule undergoes another conformational change, which deactivates the enzyme. Increased activity of phosphorylase kinase may therefore be due to increased influx of calcium ions into the cell, elevated levels of calmodulin, defective deactivation and/or elevated concentrations of the enzyme itself. The level of phosphorylase kinase is under both hormonal and neural control in the intact organism (L. C. Elliott et al., “K252a Is a Potent and Selective Inhibitor of Phosphorylase Kinase,” Biochem. Biophys. Res. Commun. 171: 148-154 (1990)). These various relationships are shown in FIG. 1.

The activity of the skin isoform of phosphorylase kinase has been shown to be present in active psoriatic epidermis (M. C. Y. Heng et al., “Elevated Phosphorylase Kinase in Psoriatic Epidermis: Correlation with Increased Phosphorylation and Glycogenolysis,” Br. J. Dermatol. 130: 298-306 (1994)). This enzyme has recently been shown to have a wider substrate specificity than previously believed (C. J. Yuan et al., “Phosphorylase Kinase, a Metal Ion-Dependent Dual Specificity Kinase,” J. Biol. Chem. 268:17683-17686 (1993)). Besides phosphorylating serine residues on glycogen phosphorylase and phosphorylase b in the presence of Mg²⁺, phosphorylase kinase has been shown to have tyrosine kinase activity in the presence of Mn²⁺ (C. J. Yuan et al. (1993), supra). In addition, phosphorylase kinase is able to phosphorylate threonine residues on troponin I (T. S. Huang et al., FEBS Lett. 42:249-252 (1974)) and even inositol in phosphatidylinositol (Z. Georgoussi & M. G. Heilmayer, Jr., “Evidence that Phosphorylase Kinase Exhibits Phosphatidylinositol Kinase Activity,” Biochemistry 25:3867-3874 (1986)), implicating the involvement of phosphorylase kinase in signaling pathways triggered by external stimuli. The α subunit of phosphorylase kinase has been shown to be homologous with regions in α-tropomyosin and human EGF receptor (T. G. Soutiroudis & T. P. Geladopoulos, “A Domain of the a-Subunit of Rabbit Phosphorylase Kinase Shows Homologies with Regions of Rabbit α-Tropomyosin, Human EGF Receptor, and the α-Chain of Bovine S-100 Protein,” Biosci. Rep. 12: 313-317 (1992)), with biological implications in the regulation of cell locomotion and cell proliferation by this enzyme.

Phosphorylase kinase is thought to link ATP production through stimulation of glycogenolysis to phosphorylation-dephosphorylation processes in calcium-calmodulin triggered hormonal-dependent signaling pathways (J. J. Davidson (1992), supra). By phosphorylating inositol to phosphoinositol (PI), phosphorylase kinase activates PI-triggered signaling pathways. By phorphorylating tyrosine kinase, phosphorylase kinase stimulates cell division and cell cycling. By phosphorylating troponin and elevating ATP levels, phosphorylase kinase affects muscle contraction and cell migration, including migration of cell types such as inflammatory cells, tumor cells, keratinocytes, and smooth muscle cells.

By taking part in ATP-dependent assembly and disassembly of actin fragments and by phosphorylating myosin to expose actin binding sites to form acto-myosin fibers, phosphorylase kinase activity is required for cell locomotion of non-muscle cells (M. F. Carlier, “Actin: Protein Structure and Filament Dynamics,” J. Biol. Chem. 266: 1-4 (1991)), including differentiating keratinocytes and inflammatory cells. Increased activity of phosphorylase kinase in psoriatic epidermis (M. C. Y. Heng et al. (1994), supra), may account, at least in part, for the increased migratory activity of inflammatory cells into uninvolved psoriatic skin following tape-stripping (M. C. Y. Heng et al., “The Sequence of Events in Psoriatic Plaque Formation After Tape-Stripping,” Br. J. Dermatol. 112: 517-532 (1995); M. C. Y. Heng et al., “Electron Microscopic and Immunocytochemical Study of the Sequence of Events in Psoriatic Plaque Formation After Tape Stripping,” Br. J. Dermatol. 125: 548-556 (1991)), and for the increased migratory activity of keratinocytes following experimental trauma (M. C. Y. Heng & S. G. Allen, “Expression of the L-Fucose Moiety in Epidermal Keratinocytes in Psoriasis Induced by the Koebner Phenomenon,” Br. J. Dermatol. 126: 575-581 (1992)).

Thus, although Applicant does not necessarily intend to be bound by this hypothesis, the control of phosphorylase kinase activity appears to have significant implications for the migration of non-muscle cells, in particular the migration of cells involved in inflammation. This migration is believed to play a key role in the propagation and maintenance of the inflammatory condition and inflammatory symptoms.

Although Applicant does not necessarily intend to be bound by this hypothesis, there is some evidence to support a hypothesis that phosphorylase kinase-mediated tyrosine kinase phosphorylation plays a role in the entry of non-cycling cells into the cell cycle. This evidence includes the following: (a) It is known that multiple phosphorylation of S6, a 40S ribosomal protein associated with the initiation of protein synthesis, is important for reentry of non-cycling cells into the cell cycle (A. M. Gressner & I. G. Wool, “The Phosphorylation of Liver Ribosomal Proteins in Vivo,” J. Biol. Chem. 249:6917-6925 (1974)). (b) Changes in calmodulin and its mRNA have been shown to accompany non-cycling (G0) cells into the early G1 phase of the cell cycle (J. G. Chafouleas et al. (1984), supra)). (c) The phosphorylation of S6 peptides has been shown to be modulated by EGF, and to involve signal transduction molecules such as cAMP (J. Martin-Perez et al., “EGF, PGF2 and Insulin Induce the Phosphorylation of Identical S6 Peptides in Swiss Mouse 3T3 Cells: Effect of cAMP on Early Sites of Phosphorylation,” Cell 36:287-294 (1984)). (d) EGF-dependent phosphorylation (J. J. Davidson et al. (1992), supra) has been shown to be triggered by calcium ions (E. J. O'Keefe & R. E. Payne, “Modulation of Epidermal Growth Factor-Receptor of Human Keratinocytes by Calcium Ion,” J. Invest Dermatol. 81:231-235 (1983)).

The role of calcium influx in inducing phosphorylase kinase activity is indicated by the fact that suppression of enzyme activity is induced by the administration of the calcium channel blocker, diltiazem, and the decreased phosphorylase kinase activity is associated with the healing phase of psoriasis. The findings of elevated levels of calmodulin in active and untreated psoriasis is consistent with its postulated role in psoriatic activity. The data discussed below shows a positive relationship between elevated levels of calmodulin and increased phosphorylase kinase activity in active and untreated psoriasis. These findings suggest that elevated calmodulin levels modulate phosphorylase kinase activity. Zinc has been shown to cause reciprocal changes in calmodulin and cAMP levels, which are in keeping with observations of inhibitory effects on calmodulin-stimulated protein kinase II on protein phosphorylation (R. P. Weinberger et al., “Effect of Zinc on Calmodulin-Stimulated Protein Kinase II and Protein Phosphorylation in Rat Cerebral Cortex,” J. Neurochem. 57:605-614 (1991)).

In addition to increased activity of the enzyme resulting from an imbalance of activators as opposed to inhibitors of phosphorylase kinase, the increased activity of the enzyme may be due to increased concentrations of the enzyme. This can be due to either increased synthesis, i.e., increased mRNA production, or decreased degradation, i.e., an increased half-life of the enzyme. An increased mRNA production can be due to an increased expression of the phosphorylase kinase gene or to the presence of multiple copies of the phosphorylase kinase gene in the genome of psoriatic individuals. The phosphorylase kinase gene in psoriatic individuals may have increased susceptibility to induction by viral oncogenes or proto-oncogenes induced by cytokines, thus providing an explanation for the role of the lymphocyte-mediated immune response and the role of external antigens in psoriasis. This may account for the sensitivity of the disease to external environmental factors. The possibility that the psoriatic phosphorylase kinase gene may be inducible by cytokines, with the resulting epidermal proliferation modified by growth factors and their receptors is suggested by the association of psoriasis with T-cell-mediated responses, with resultant cytokine secretions such as IL-8, tumor necrosis factor, and interferon-γ. The resulting enhanced production of growth factors such as transforming growth factor-α and of its ligand, epidermal growth factor receptor, appears to be involved in the hyperproliferative manifestations of the disease.

Two psoriatic susceptibility loci have been identified in the human genome, at 17q and 16q (R. P. Nair et al., “Evidence for Two Psoriatic Susceptibility Loci (HLA and 17q) and Two Novel Candidate Regions (16Q and 20p) by Genome-Wide Scan,” Hum. Mol. Genet. 6:1349-1356 (1997)) The locus at 17q may be linked to cAMP-dependent protein kinase type 1. The locus at 16q, as indicated above, is linked to the β subunit of phosphorylase kinase (Francke et al. (1989), supra).

Although these mechanisms of injury are particular to psoriasis, similar mechanisms are likely to operate in the inflammation accompanying wounds, burns, acne, and eczema, as well as skin damage, including premature aging, resulting from exposure to sunlight and tobacco smoke/nicotine. Thus, agents that decrease phosphorylase kinase activity, such as curcumin, are likely to be effective in preventing inflammation accompanying these conditions and, consequently, their resultant destructive sequelae. Additionally, given the role of PK in cellular proliferation, agents that can decrease PK activity are also useful in blocking cellular proliferation. This provides a basis for the rational therapy of not only inflammatory conditions, but also of inflammatory cytokine-aggravated malignancies and of infections. One mechanism for this is the activity of PK inhibitors such as curcumin in inhibiting ligand-induced activation of epidermal growth factor receptor tyrosine phosphorylation (L. Korutla et al., “Inhibition of Ligand-Induced Activation of Epidermal Growth Factor Receptor Tyrosine Phosphorylation by Curcumin,” Carcinogenesis 16: 1741-1745 (1995)). Another mechanism for this is the downregulation of chemokine expression in cells such as bone marrow stromal cells (Y. X. Xu et al., “Curcumin, a Compound with Anti-Inflammatory and Anti-Oxidant Properties, Down-Regulates Chemokine Expression in Bone Marrow Stromal Cells,” Exp. Hematol. 25: 413-422 (1997)).

Similar mechanisms are likely to exist in other conditions that can be affected by the modulation of phosphorylase kinase activity. These conditions include diabetes (H. Liu & J. H. McNeill, “Effects of Vanadium Treatment on the Alterations of Cardiac Glycogen Phosphorylase and Phosphorylase Kinase in Streptozotocin-Induced Chronic Diabetic Rats,” Can. J. Physiol. Pharmacol. 72: 1537-1543 (1994); T. G. Sotiroudis & T. P. Geladopoulos, “A Domain of the cc-Subunit of Rabbit Phosphorylase Kinase Shows Homologies with Regions of Rabbit α-Tropomyosin, Human EGF Receptor, and the cc Chain of Bovine S-100 Protein,” Biosci. Rep. 12: 313-317 (1992)). These conditions also include injury from consumption of alcohol or consumption or administration of drugs of abuse (S. W. French, “The Mechanism of Organ Injury to Alcoholics: Implications for Therapy,” Alcohol. Alcohol. Suppl. 1: 57-63 (1991)). These conditions further include neurodegenerative conditions such as Alzheimer's disease (C. G. Gong et al., “Phosphoprotein Phosphatase Activities in Alzheimer Disease Brain,” J. Neurochem. 61: 921-927 (1993); H. K. Paudel et al., “Phosphorylase Kinase Phosphorylates the Calmodulin-Binding Regulatory Regions of Neuronal Tissue-Specific Proteins B-50 (GAP-43) and Neurogranin,” J. Biol. Chem. 268: 6207-6213 (1993); H. K. Paudel, “The Regulatory Ser262 of Microtubule-Associated Protein τ Is Phosphorylated by Phosphorylase Kinase,” J. Biol. Chem. 272: 1777-1785 (1997); C. X. Gong et al., “Inhibition of Protein Phosphatase-2B (Calcineurin) Activity Towards Alzheimer Abnormally Phosphorylated τ by Neuroleptics,” Brain Res. 74: 95-102 (1996).

Accordingly, the present invention encompasses methods for inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription. These methods are directed to treating, controlling, or preventing inflammation and its sequelae, as discussed above.

II. Active Agents and Compositions for Administering them

A. Curcumin

Curcumin has the structure shown in (I)

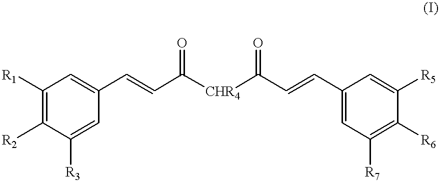

where R₁ is —OCH₃; R₂ is —OH; R₃ is —H; R₄ is —H; R₅ is —OCH₃; R₆ is —OH, and R₇ is H. Curcumin has the chemical name (E, E) 1,7-bis(4-hydroxy-3-methoxyphenyl)-1,6-heptadiene-3,5-dione. In natural curcumin, the carbon-carbon double bonds are in the trans configuration.

Curcumin (diferuloylmethane) is a major active component of the food flavor, turmeric (Curcuma longa; S. Reddy S & B. B. Aggarwal, “Curcumin Is a Non-Competitive and Selective inhibitor of Phosphorylase Kinase,” FEBS Lett. 341:19-22 (1994)). The anti-proliferative properties of curcumin in animals has been demonstrated by its inhibition of tumor initiation induced by benzo[a]pyrene and 7,12 dimethylbenz[a]anthracene (M. T. Huang et al., Carcinogenesis 13:2183-2186 (1992); M. A. Azuine & S. V. Bhide, Nutr. Cancer 17:77-83 (1992); M. A. Azuine & S. V. Bhide, Int. J. Cancer 51:412-415 (1992); M. Nagabhushan & S. V. Bhide, J. Am. Coll. Nutr. 11: 192-198 (1992)), and inhibition of populations of various cell types (H. P. Ammon & M. A. Wahl, Planta Med. 57:1-7 (1991); R. R. Satoskar et al., Int. J. Clin. Pharmacol. Res. 24:651-654 (1986); T. N. B. Shankar et al., Indian J. Exp. Biol. 18:73-75 (1980); R. Srivastava, Agents Action 38:298-303 (1989); H. C. Huang et al., Eur. J. Pharmacol. 221:381-384 (1992)). In addition, curcumin inhibits the tumor promotion caused by phorbol esters (M. T. Huang et al., Cancer Res. 48:5941-5946 (1988); A. H. Conney et al., Adv. Enzyme Regul. 31: 385-396 (1991); Y. P. Lu et al., “Effect of Curcumin on 12-O-Tetradecanoylphorbol-13-Acetate- and Ultraviolet B Light-Induced Expression of c-Jun and c-Fos in JB6 Cells and in Mouse Epidermis,” Carcinogenesis 15: 2263-2270 (1994)). Recently, curcumin has been shown to inhibit pp60src (epidermal growth factor equivalent) tyrosine kinase via inhibition of phosphorylase kinase (S. Reddy & B. B. Aggarwal (1994), supra). It is possible that both the anti-tumor and anti-proliferative properties of curcumin may be mediated via its selective and non-competitive inhibition of phosphorylase kinase (S. Reddy & B. B. Aggarwal (1994), supra).

Curcumin is an inhibitor of Type I cyclic AMP-dependent protein kinase, the enzyme mainly responsible for activating phosphorylase kinase. The inhibition is competitive with respect to both ATP and the substrate (M. Hasmeda & G. M. Polya, “Inhibition of Cyclic AMP-Dependent Protein Kinase by Curcumin,” Phytochemistry 42: 599-605 (1996)). Phosphorylase kinase, in turn, increases the migration of inflammatory cells, tumor cells, smooth muscle cells, and other cell types, as discussed above, as well as infectious organisms, increasing both the destructive and proliferative sequelae of the inflammatory response.

Accordingly, an improved method of treatment of wounds, burns, acne, and eczema, as well as skin damage resulting from exposure to sunlight or exposure to cigarette smoke or nicotine, utilizes inhibition of phosphorylase kinase activity in the affected skin in a mammal, particularly a human. A particularly suitable reagent for inhibiting phosphorylase kinase activity is curcumin.

Similarly, an improved method of treatment of inflammation utilizes inhibition of phosphorylase kinase activity in a mammal suffering from inflammation. The method comprises the step of treating the mammal affected with inflammation with curcumin in a quantity sufficient to detectably inhibit phosphorylase kinase activity. The method addresses and ameliorates the systemic consequences of inflammation as well as many of the dermatological and pathological consequences. In particular, this method ameliorates the effect of inflammatory changes occurring in the vascular system as the consequence of such conditions as hypoxia, ischemia, or exposure to cigarette smoke.

Curcumin is administered in a quantity sufficient to reduce the activity of phosphorylase kinase as measured by phosphorylation of a suitable substrate. Typically, phosphorylase kinase activity is measured by determining the conversion rate of phosphorylase b to phosphorylase a, measuring radioactive phosphate transferred from [³²P]ATP to the phosphorylase b. Other assay methods are also known to the art.

The method of the present invention is effective in preventing inflammation in the affected epidermis, in drying the lesions, and in promoting healing without excessive scar formation. Similarly, the method of the present invention is effective in preventing the systemic consequences of inflammation, including the effects of inflammation on the vascular system.

The dosages to be administered can be determined by one of ordinary skill in the art depending on the clinical severity of the disease, the age and weight of the patient, the exposure of the patient to conditions that may precipitate outbreaks of psoriasis or other dermatological or systemic inflammatory conditions, or other conditions that modulate the activity of phosphorylase kinase, the degree of exposure to such conditions as sunlight or tobacco smoke, and other pharmacokinetic factors generally understood in the art, such as liver and kidney metabolism. The interrelationship of dosages for animals of various sizes and species and humans based on mg/m³ of surface area is described by E. J. Freireich et al., “Quantitative Comparison of Toxicity of Anticancer Agents in Mouse, Rat, Hamster, Dog, Monkey and Man,” Cancer Chemother. Rep. 50: 219-244 (1966). Adjustments in the dosage regimen can be made to optimize the therapeutic response. Doses can be divided and administered on a daily basis or the dose can be reduced proportionally depending on the therapeutic situation.

Typically, curcumin is administered topically, particularly for skin and mucosal diseases; alternatively, it can be administered in conventional pill or liquid form for treatment of severe skin and systemic disease. If administered in pill form, it can be administered in conventional formulations with excipients, fillers, preservatives, and other typical ingredients used in pharmaceutical formations in pill form. Typically, curcumin is administered in a conventional pharmaceutically acceptable formulation, typically including a carrier. Conventional pharmaceutically acceptable carriers known in the art can include alcohols, e.g., ethyl alcohol, serum proteins, cholesterol, human serum albumin, liposomes, buffers such as phosphates, water, sterile saline or other salts, electrolytes, glycerol, hydroxymethylcellulose, propylene glycol, polyethylene glycol, polyoxyethylenesorbitan, other surface active agents, vegetable oils, and conventional anti-bacterial or anti-fungal agents, such as parabens, chlorobutanol, phenol, sorbic acid, thimerosal, and the like. A pharmaceutically-acceptable carrier within the scope of the present invention meets industry standards for sterility, isotonicity, stability, and non-pyrogenicity.

The pharmaceutically acceptable formulation can also be in pill, tablet, or lozenge form as is known in the art, and can include excipients or other ingredients for greater stability or acceptability. For the tablets, the excipients can be inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc, along with the curcumin, curcumin derivatives, or curcuminoids, the substances, such as alcohols, for controlling the solubility of the curcumin, curcumin derivatives, or curcuminoids, and other ingredients.

Curcumin can also be administered in liquid form in conventional formulations, that can include preservatives, stabilizers, coloring, flavoring, and other generally accepted pharmaceutical ingredients. Typically, when curcumin is administered in liquid form, it is in alcoholic solution, which can also contain water. The alcoholic solution typically contains alcohols such as ethyl alcohol or other pharmaceutically tolerated compounds, and can contain buffers. As discussed below, it is particularly preferred to administer curcumin in a solution containing an alcohol.

Alternatively, curcumin can be administered by injection by one of several routes well known in the art. It is, however, generally preferred for the treatment of skin conditions, to administer curcumin topically, such as in a 1% gel. Typically, the 1% curcumin gel is in an aloe vera base and contains at least one alcohol, as described below. For topical applications, preferably, the alcohol is ethyl alcohol or isopropyl alcohol. For systemic administration, most preferably, the alcohol is ethyl alcohol. Other suitable cosmetic carriers, excipients, stabilizers, other conventional ingredients used in pharmaceutical gels, and the like can also be present. Formulations for topical gels suitable for administration of curcumin are well known in the art; one suitable formulation uses an aloe vera gel base, as indicated above. A particularly suitable aloe vera-containing gel base contains aloe vera, ethanol, glycerol, triethanolamine-carbomer 940, tetrasodium EDTA, benzophenone-4, and sodium hydroxymethylglycinate.

Curcumin can be administered from once per day to up to at least five times per day, depending on the severity of the disease, the total dosage to be administered, and the judgment of the treating physician. In some cases, curcumin need not be administered on a daily basis, but can be administered every other day, every third day, or on other such schedules. However, it is generally preferred to administer curcumin daily.

Typically, curcumin is administered orally in a dose of about 250 mg to about 2 g daily, or in a topical gel at about 0.1% to about 2% concentration. However, curcumin can also be administered in dosages outside these ranges, as appropriate for the particular patient and condition.

Curcumin can be administered alone, or, as described further below, in combination with other drugs.

Curcumin can be administered by a variety of routes, including orally or as a gargle for the throat, topically for the skin and the mucous membranes, intraocularly for the eye, intraaurally for the ear, intranasally for the nose and the nasal sinuses, or by intraesophageal, intragastric, intestinal, anal, colonic, intravaginal, intramuscular, intrauterine, intra-bladder, intraureter, intraurethral, or parenteral (intravenous or intraperitoneal) routes according to the dosage desired, the nature of the condition to be treated, and the response of the patient. Soluble curcumin can also be administered locally on the gingiva by local injection for dental diseases.

It is particularly preferred to administer curcumin in an alcoholic solution or a gel base containing an alcohol. The term “alcohol” as used herein refers to a lower alcohol, typically an alcohol of 1 to 12 carbon atoms, preferably an alcohol of 1 to 6 carbon atoms, more preferably an alcohol of 2 or 3 carbon atoms. The alcohol can be saturated or unsaturated; preferably it is saturated. The alcohol can be monohydric or polyhydric; preferably it is monohydric. The alcohol can contain other substituents such as halo, carboxylic acid, or nitro, and can be cyclic; however, these alternatives are not generally preferred. Particularly preferred alcohols are ethanol, 1-propanol, and 2-propanol (isopropyl alcohol); a most particularly preferred alcohol is ethanol.

Preferably, the concentration of alcohol in the solution or gel base is at least about 1%. When the curcumin is administered in gel form, the concentration of alcohol is preferably is about 10-30%. Alternatively, when the curcumin is administered in capsule form, the concentration of alcohol is preferably about 50-80%.

In one embodiment according to the present invention, the solution or gel base in which the curcumin is administered contains an antioxidant. The antioxidant can be selected from the group consisting of reduced glutathione, N-acetyl-L-cysteine, β-carotene, or ascorbic acid (S. Oetari et al., “Effects of Curcumin on Cytochrome P450 and Glutathione S-Transferase Activities in Rat Liver,” Biochem. Pharmacol. 51: 39-45 (1995)). Alternatively, the antioxidant can be a free radical quencher such as catalase or superoxide dismutase.

In another embodiment according to the present invention, the solution or gel base in which the curcumin is administered is packaged in a liposome, such as a phosphatidyl choline liposome (D. V. Rajakumar & M. N. Rao, “Antioxidant Properties of Phenyl Styryl Ketones,” Free Radical Res. 22: 309-317 (1995)). The preparation and use of liposomes to deliver drugs and other physiologically active compounds is well known in the art and need not be described further here. Phosphorylase kinase inhibitors according to the present invention, including curcumin, curcumin derivatives, and curcuminoids, can be packaged in liposomes in skin preparations for cutaneous administration. Alternatively, they can be packaged for administration to mucous membranes, such as in eye drops, nasal drops, oral or pharyngeal preparations, rectal or vaginal preparations, or bladder or urethral preparations. As another alternative, they can be packaged for administration as bronchial preparations.

In yet another embodiment according to the present invention, the curcumin, curcumin derivative, or curcuminoid is administered in the form of a boron complex (Z. Sui et al., “Inhibition of the HIV-1 and HIV-2 Proteases by Curcumin and Curcumin Boron Complexes,” Bioorg. & Med. Chem. 1: 415-422 (1993)). These boron complexes of curcumin, curcumin derivatives, or curcuminoids can include, but are not necessarily limited to, difluoroboron complexes and mixed complexes in which the two fluorine atoms of difluoroboron complexes are replaced with: (1) the carboxyl oxygens of oxalic acid; (2) a carboxyl group and a hydroxyl group of citric acid; (3) the two hydroxyl groups of dibenzyl tartramide; or (4) a second molecule of curcumin, a curcumin derivative, or a curcuminoid.

B. Curcuminoids and Derivatives of Curcumin

Also within the scope of the present invention is the use of curcumin derivatives or curcuminoids in place of curcumin itself or in addition to curcumin.

Among the curcuminoids that can be used in methods according to the present invention are curcuminoids of formula (I) with the following alternative combinations of substituents:

(A) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;

(B) R₁ is —H; R₂ is —OH; R₃ is —H or —OH; R₄ is —H, R₅ is —H; R₆ is —OH; and R₇is —H or —OH;

(C) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;

(D) R₁ is —OH, R₂ is —OH, R₃ is —OH, R₄ is —H or —OH, R₅ is —OH, R₆ is —OH; and R₇ is —OH;

(E) R₁ is —OCH₃; R₂ is —OCH₃; R₃ is —OCH₃; R₄ is —H or —OH; R₅ is —OCH₃; R₆ is —OCH₃; and R₇ is —OCH₃;

(F) R₁ is —H; R₂ is —OCH₃; R₃ is —OCH₃; R₄ is —H or —OH; R₅ is —H; R₆ is —OCH₃; and R₇ is —OCH₃;

(G) R₁ is —H; R₂ is —OH; R₃ is —H; R₄ is —H; R₅ is —H or —OH; R₆ is —OH; and R₇ is —H;

(H) R₁ is —H; R₂ is —OCH₃; R₃ is —H; R₄ is —H; R₅ is —H or —OH; R₆ is —OCH₃; and R₇ is —H; or

(I) R₁ is —OH; R₂ is —OCH₃; R₃ is —H or —OH; R₄ is H or —OH; R₅ is —OH; R₆ is —OCH₃; and R₇ is —H or —OH.

Among the additional curcuminoids that can be used in methods according to the present invention are curcuminoids of formula (II):

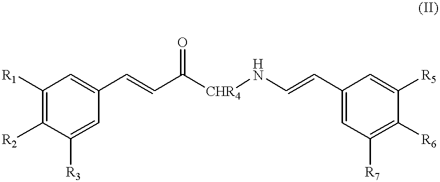

in which R₁ through R₇ can be as in curcumin or as in alternatives (A) through (1) above.

Also among the curcuminoids that can be used in methods according to the present invention are curcuminoids of formula (III):

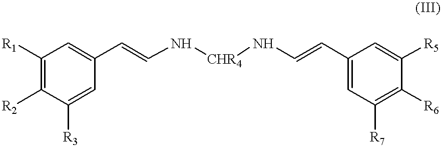

in which R₁ through R₇ can be as in curcumin or as in alternatives (A) through (I) above.

Additionally, among the curcuminoids that can be used in methods according to the present invention are analogues of curcuminoids of formulas (I) or (II) in which either or both of the oxygens of the carbonyl (CO) groups are replaced with sulfur to form a thiocarbonyl (CS) group.

Also among the curcuminoids that can be used in methods according to the present invention are furfural curcuminoid (formula IV) (X=—H); derivatives of furfural curcuminoid in which X is —OH, ethyl, methyl, or acetyl; salicyl curcuminoid (formula V) (X=—H), derivatives of salicyl curcuminoid in which X is —OH, ethyl, methyl, or acetyl; veratryl curcuminoid (formula VI) (X=—H); derivatives of veratryl curcuminoid in which X is —OH, ethyl, methyl, or acetyl; p-anisyl curcuminoid (formula VII) (X=—H); derivatives of p-anisyl curcuminoid in which X is —OH, ethyl, methyl, or acetyl; and piperonal curcuminoid (formula VIII) (X=—H); and derivatives of piperonal curcuminoid in which X is —OH, ethyl, methyl, or acetyl (R. J. Anto et al., “Antimutagenic and Anticarcinogenic Activity of Natural and Synthetic Curcuminoids,” Mutation Res. 370: 127-131 (1996)). Additionally among the curcuminoids that can be used in methods according to the present invention are compounds that are analogues to the curcuminoids of formulas IV through VIII in which one or both of the carbonyl (CO) groups are replaced by amino (NH) groups in analogy with formulas II and III, or in which one or both of the oxygens of the carbonyl groups are replaced by sulfur to form thiocarbonyl groups.

Additionally among the curcuminoids that can be used in methods according to the present invention are tetrahydrocurcuminoids of formula IX produced by reducing curcuminoids by hydrogenation with a PtO₂ catalyst, in which R₁ through R₇ can be as in curcumin or as in alternatives (A) through (I) above. Additionally among the curcuminoids that can be used in methods according to the present invention are compounds that are analogues to the curcuminoids of formula IX in which one or both of the carbonyl (CO) groups are replaced by amino (NH) groups in analogy with formulas II and III, or in which one or both of the oxygens of the carbonyl groups are replaced by sulfur to form thiocarbonyl groups.

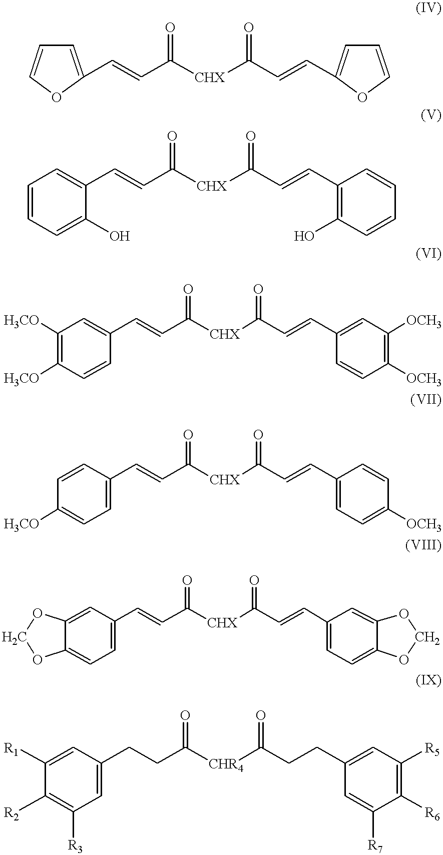

(T. Osawa et al., “Antioxidative Activity of Tetrahydrocurcuminoids,” Biosci. Biotech. Biochem. 59: 1609-1612 (1995)).

In natural curcumin, the stereochemistry at each of the double bonds is E (trans), so the molecule has the stereochemistry (E,E). However, the other geometrical isomers (Z, Z), (Z, E), and (E, Z), where these last two exist separately because of the nonsymmetric nature of the molecule, also can be prepared, and the use of these geometrical isomers is within the scope of the present invention. This includes the geometrical isomers of any of the molecules represented by Formulas I through IX, and recitation of these formulas in the specification and the claims of the present application includes both cis and trans geometrical isomers unless one of the geometrical isomers is explicitly specified.

Additionally, within the scope of the present invention are tautomers of the above structures in which one or both of the keto moieties located at the center portion of the molecule are replaced with enol moieties. These include the following: (1) molecules of Formula X in which in which R₁ through R₇ can be as in curcumin or as in alternatives (A) through (I) above; and (2) molecules of Formula XI in which R₁ through R₇ can be as in curcumin or as in alternatives (A) through (I) above. In Formulas X and XI, the double bonds are in the trans configuration, but also within the scope of the present invention are molecules in which one or more of the double bonds are in the cis configuration, as indicated above; for these formulas as well, their recitation in the specification and the claims of the present application includes both cis and trans geometrical isomers unless one of the geometrical isomers is explicitly specified. Additionally, among the curcuminoids that can be used in methods according to the present invention are analogues of curcuminoids according to formula (X) in which the carbonyl (CO) group is replaced with an amino (NH) group or in which the oxygen of the carbonyl group is replaced with a sulfur atom.

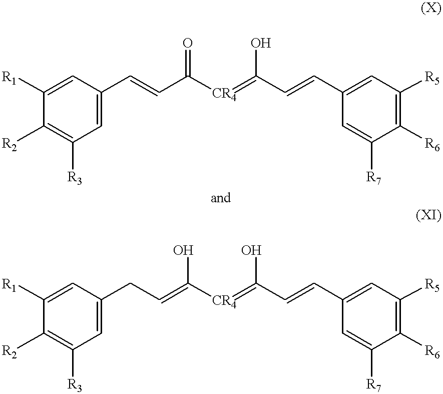

Additionally, within the scope of the present invention are reduced derivatives of curcumin represented by Formula XII in which R₁ through R₇ can be as in curcumin or as in alternatives (A) through (1) above.

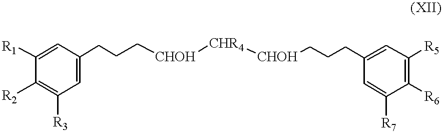

Also within the scope of the present invention are derivatives of curcumin or curcuminoids in which any of the methoxy groups are replaced with lower alkoxy groups such as ethoxy, n-propoxy, or isopropoxy. Additionally, within the scope of the present invention are derivatives of curcumin or curcuminoids in which any of the phenolic hydroxy groups in the structure are acylated with acyl substitutents such as acetyl, propionyl, butyryl, or isobutyryl (Sreejayan & M. N. Rao, “Curcuminoids as Potent Inhibitors of Lipid Peroxidation,” J. Pharm. Pharmacol. 46: 1013-1016 (1994)).

Also, additionally within the scope of the present invention are derivatives of curcumin or corcuminoids in which the hydrogens of one or more of the —OH groups present are replaced by an alkali metal, preferably sodium. When the hydrogens of the —OH groups in curcumin are replaced by sodium, the result is sodium curcuminate.

These curcuminoids and curcuminoid derivatives are administered in the same way as described above for curcumin. Preferably, these compounds are administered in a solution or gel base containing an alcohol, as described above.

III. Use of Curcumin, Curcumin Derivatives, or Curcuminoids Along with Other Compounds

Another aspect of the present invention is the use of curcumin, curcumin derivatives, or curcuminoids along with one or more additional compounds. These additional compounds can include the following:

(1) vitamin D₃ or vitamin D₃ analogues such as calcipotriol, calcipotriene, or 1α,24-dihydroxyvitamin D₃;

(2) vitamin A or vitamin A derivatives or analogues such as P-carotene or retinoids;

(3) calmodulin inhibitors such as cyclosporin A, zinc, anthralin, or trifluoroperazine;

(4) other anti-inflammatory drugs such as corticosteroids, substance P inhibitors such as capsaicin, capsaicin-sensitive vanilloid receptor inhibitors such as capsazepine, cyclo-oxygenase inhibitors such as acetylsalicylic acid, and other non-steroidal anti-inflammatory agents such as naproxen;

(5) calcium channel blockers such as diltiazem;

(6) H1 and H2 histamine blockers;

(7) vitamin E (α-tocopherol) and other antioxidants and free radical quenchers such as β-carotene, reduced glutathione, superoxide dismutase, and catalase;

(8) polyphenolic compounds such as rutin, catechin, epicatechin, naringin, naringenin, gallotanin and epigallotanin;

(9) monoterpenes;

(10) genistein;

(11) soybean derived lectins such as soybean agglutinin; and

(12) dehydrozingerone.

These additional compounds can be administered separately but simultaneously with curcumin, curcumin derivatives, or curcuminoids, or can be administered in a combined formulation with curcumin, curcumin derivatives, or curcuminoids.

A. Vitamin D₃ and Analogues of Vitamin D₃

Vitamin D₃ and its analogues are cAMP-dependent protein kinase II activators. Typically, a vitamin D₃ analogue such as 1α,25-dihydroxy vitamin D₃, also known as calcitriol (M. J. Gerritsen et al., “Transglutaminase-Positive Cells in Psoriatic Epidermis During Treatment with Calcitriol (1α,25 dihydroxy vitamin D₃) and Tacalcitol (1α,24 dihydroxy vitamin D₃),” Br. J. Dermatol. 133: 656-659 (1995)) is used. Another suitable analogue is calcipotriene, which can be is administered at a concentration of about 0.005% in the form of an ointment. An available calcipotriene ointment that is suitable is Donovex; it can be administered twice daily. These vitamin D₃ analogues can be administered orally or by additional routes. In many applications, vitamin D₃ analogues are preferred additional compounds because of their lack of toxicity. Other vitamin D₃ analogues usable in methods according to the present invention include calcipotriol (J. Reichrath et al., “Biologic Effects of Topical Calcipotriol (MC 903) Treatment in Psoriatic Skin,” J. Am. Acad. Dermatol. 36: 19-28 (1997)), and tacalcitol (1α,24-dihydroxy vitamin D₃), as well as derivatives of these compounds. Other analogues and derivatives of vitamin D₃ are known in the art and can be used in methods according to the present invention.

B. Vitamin A and its Derivatives and Analogues

Vitamin A and its derivatives and analogues, such as retinoids and β-carotene, can be administered as additional compounds. These compounds are also active as cAMP-dependent protein kinase II activators. Vitamin A and its derivatives and analogues can be administered orally or by other routes. An example of a vitamin A analogue is tazarotene. Tazarotene can be administered in a topical gel at a concentration of from about 0.001% to about 1% once to three times daily. Preferred concentrations of tazarotene in the topical gel are about 0.05% and about 0.1%. Although Applicant does not intend to be bound by this theory, one possible mechanism of action of retinoids is to reverse a defect or defects in cAMP-dependent protein kinases, namely in the regulatory subunits (S. Tournier et al., “Retinoylation of the Type II cAMP-Binding Regulatory Subunit of cAMP-Dependent Protein Kinase Is Increased in Psoriatic Human Fibroblasts,” J. Cell. Physiol. 167: 196-203 (1996); S. Tournier et al., “Post-Translational Abnormality of the Type II Cyclic AMP-Dependent Protein Kinase in Psoriasis: Modulation by Retinoic Acid,” J. Cell. Biochem. 57: 647-654 (1995)).

C. Calmodulin Inhibitors

Calmodulin inhibitors include zinc, cyclosporin, anthralin, and trifluoroperazine (N. Bouquin et al., “Resistance to Trifluoroperazine, a Calmodulin Inhibitor, Maps to the fabD Locus in Escherichia coli,” Mol. Gen. Genet. 246: 628-637 (1995)). Anthralin can be administered in the form of an ointment or paste at a concentration of from about 0.1% to about 3% once or more daily, typically once or twice daily. Cyclosporin can be administered orally or by other routes.

D. Other Anti-inflammatory Drugs

Other anti-inflammatory drugs can be used as additional compounds. These include: (1) corticosteroids; (2) substance P inhibitors such as capsaicin; (3) capsaicin-sensitive vanilloid receptor inhibitors such as capsazepine; (4) cyclo-oxygenase inhibitors such as acetylsalicylic acid, and (5) other non-steroidal anti-inflammatory agents such as naproxen.

1. Corticosteroids

Corticosteroids are well-known as anti-inflammatory agents. Among the corticosteroids with anti-inflammatory activity are cortisone and its derivatives and salts such as cortisone acetate, hydrocortisone and its derivatives and salts such as hydrocortisone acetate, hydrocortisone cypionate, hydrocortamate hydrochloride, hydrocortisone sodium succinate, hydrocortisone sodium phosphate, fludrocortisone and its derivatives and analogues such as fludrocortisone acetate and the 9α-bromo analogue of fludrocortisone, prednisone, prednisolone, prednisolone acetate, prednisolone t-butylacetate, prednisolone sodium phosphate, methylprednisolone, methylprednisolone 21-acetate, methylprednisolone sodium succinate, triamcinolone, triamcinolone acetonide, dexamethasone, betamethasone, paramethasone, fluprednisolone, flurandrenolone, fluorometholone, fluocinolone, and their derivatives. Other anti-inflammatory steroids are well known in the art. These anti-inflammatory steroids can be administered orally, by intravenous or intradermal injection, topically, and by other routes.

2. Substance P Inhibitors

Another class of compounds that can be used as additional compounds in methods according to the present invention is Substance P inhibitors such as capsaicin. Substance P is an 11-residue peptide that is derived from protachykinin β precursor and is a tachykinin. Capsaicin has been shown to have anti-inflammatory activity, lowers Ca²⁺, Mg²⁺-ATPase activity associated with macrophage membranes, and acts as an inhibitor of the generation of reactive oxygen and nitrogen intermediates by macrophages (B. Joe & B. R. Lokesh, “Role of Capsaicin, Curcumin and Dietary n-3 Fatty Acids in Lowering the Generation of Reactive Oxygen Species in Rat Peritoneal Macrophages,” Biochim. Biophys. Acta 1224: 255-263 (1994); T. Biro et al., “Recent Advances in Understanding of Vanilloid Receptors: A Therapeutic Target for Treatment of Pain and Inflammation in Skin,” J. Invest. Dermatol. Symp. Proc. 2: 56-60 (1997)). Capsaicin also modulates the presence of an acidic glycoprotein that is characteristic of an inflammatory response in the serum of rats with adjuvant induced arthritis (B. Joe et al., “Presence of an Acidic Glycoprotein in the Serum of Arthritic Rats: Modulation by Capsaicin and Curcumin,” Mol. Cell. Biol. 169: 125-134 (1997)) Capsaicin can be administered orally, topically, and by other routes.

In addition to capsaicin, other additional compounds include the capsaicin analogue resiniferatoxin (T. Biro et al. (1997), supra) and such capsaicin analogues as substituted benzylnonanamides, N-octyl-substituted phenylacetamides, N-(4-hydroxy-3-methoxybenzyl)-N′-octylthiourea, and vanillylamides and vanillylthioureas with hydrophobic side chains (C. S. J. Walpole, “Analogues of Capsaicin with Agonist Activity as Novel Analgesic Agents; Structure-Activity Studies. 1. The Aromatic ‘A-Region,’” J. Med. Chem. 36: 2362-2372 (1993); C. S. J. Walpole, “Analogues of Capsaicin with Agonist Activity as Novel Analgesic Agents; Structure-Activity Studies. 2. The Amide Bond ‘B-Region,’” J. Med. Chem. 36: 2373-2380 (1993); C. S. J. Walpole, “Analogues of Capsaicin with Agonist Activity as Novel Analgesic Agents; Structure-Activity Studies. 3. The Hydrophobic Side-Chain ‘C.-Region,’” J. Med. Chem 36: 2381-2389 (1993)).

3. Capsaicin-sensitive Vanilloid Receptor Inhibitors

Another class of additional compounds is capsaicin-sensitive vanilloid receptor inhibitors such as capsazepine, which acts as an antagonist of capsaicin. (S. Bevan et al., “Capsazepine: A Competitive Antagonist of the Sensory Neurone Excitant Capsaicin,” Br. J. Pharmacol. 107: 544-552 (1992); T. Ohkubo & K. Kitamura, “Eugenol Activates Ca²⁺-Permeable Currents in Rat Dorsal Root Ganglion Cells,” J. Dent. Res. 76: 1737-1744 (1997); T. Ohkubo & M. Shibata, “The Selective Capsaicin Antagonist Capsazepine Abolishes the Antinociceptive Action of Eugenol and Guaiacol,” J. Dent. Res. 76: 848-851 (1997)). Capsazepine can be administered orally and by other routes.

4. Acetylsalicylic Acid and Other Cyclo-oxygenase Inhibitors

Another class of additional compounds that can be used in methods according to the present invention is acetylsalicylic acid and other cyclo-oxygenase inhibitors (I and II) (Cox I and II inhibitors). These compounds inhibit the production of prostaglandins, thromboxanes, and leukotrienes by inhibiting the activity of the cyclo-oxygenase enzymes. They exhibit anti-inflammatory activity. In addition to acetylsalicylic acid, related compounds can be used in methods according to the present invention, such as sodium salicylate, choline salicylate, salicylamide, salsalate, 3-methylacetylsalicylic acid, 3-methylsalicylic acid, 5-(2,4-difluorophenyl)salicylic acid, and benorylate. These compounds can be administered orally, topically, and by other routes.

5. Other Non-steroidal Anti-inflammatory Agents

Another class of additional compounds that can be used in methods according to the present invention is other non-steroidal anti-inflammatory agents. Like acetylsalicylic acid and its derivatives, these compounds also are believed to suppress inflammation by inhibiting the activity of the cyclo-oxygenase enzyme. These compounds include antipyrine, phenylbutazone, oxyphenbutazone, sulfinpyrazone, mefenamic acid, meclofenamic acid, flufenamic acid, indomethacin, sulindac, tolmetin, zomepirac, ibuprofen, fenoprofen, ketoprofen, suprofen, naproxen, piroxecam, and other compounds. These compounds are typically administered orally, but can be administered by other routes.

E. Calcium-channel Blockers

Another class of additional compounds useful in methods according to the present invention is calcium-channel blockers such as diltiazem. Diltiazem can be administered orally in a dose of from about 30 mg three times daily to about 90 mg three times daily. A preferred dose of dilitiazem is 60 mg three times daily. The equivalent dose can be given in a long-acting preparation once or twice daily. Other calcium-channel blockers can be used. These include nifedepine, isradipine, and verapamil.

F. H1 and H2 Histamine Blockers

Another class of additional compounds useful in methods according to the present invention is H1 and H2 histamine blockers. Many such compounds are known. Representative H1 histamine blockers include carbinoxamine maleate, clemastine fumarate, diphenhydramine hydrochloride, dimenhydrinate, pyrilamine maleate, tripelennamine hydrochloride, tripelennamine citrate, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, acrivastine, cetirizine hydrochloride, astemizole, levocabastine hydrochloride, loratadine, and terfenadine. Representative H2 histamine blockers include cimetidine, ranitidine, famotidine, and nizatidine. Both H1 and H2 histamine blockers are typically administered orally or topically; other routes of administration are possible. The dosages and routes of administration of these histamine blockers are described in J. G. Hardman & L. E. Limbird, eds., “Goodman & Gilman's The Pharmacological Basis of Therapeutics” (9th ed., McGraw-Hill, New York, 1996), pp. 581-600, incorporated herein by this reference.

G. Vitamin E (α-tocopherol) and Other Anti-oxidants

Another class of additional compounds that can be used in methods according to the present invention is vitamin E (α-tocopherol) and other anti-oxidants. These compounds can be administered orally, topically, and by other routes. Anti-oxidants also include β-carotene, whose use is described above.

H. Polyphenolic Compounds

Another class of additional compounds that can be used in methods according to the present invention is polyphenolic compounds. This class of compounds includes (−)epigallocatechin-3-gallate, rutin, catechin, epicatechin, naringin, naringenin, and gallotanin (L. G. Menon et al., “Inhibition of Lung Metastasis in Mice Induced by B 16F10 Melanoma Cells by Polyphenolic Compounds,” Cancer Lett. 95: 221-225 (1995); G. D. Stoner & H. Mukhtar, “Polyphenols as Cancer Preventative Agents,” J. Cell. Biochem. Suppl. 22:169-180 (1995); M. M. Chan et al., “Effects of Three Dietary Phytochemicals from Tea, Rosemary and Turmeric on Inflammation-Induced Nitrite Production,” Cancer Lett. 96: 23-29 (1995)). These polyphenolic compounds can be administered orally or by other routes.

I. Monoterpenes

Another class of additional compounds useful in methods according to the present invention is monoterpenes such as d-limonene and perillyl alcohol (X. Chen et al., “Inhibition of Farnesyl Protein Transferase by Monoterpene, Curcumin Derivatives and Gallotannin,” Anticancer Res. 17:2555-2564 (1997)). These compounds inhibit the enzyme farnesyl protein transferase, which is crucial in the isoprenylation of the Ras proteins. These compounds can be administered orally, intravenously, and by other routes.

J. Genistein

Another compound useful in methods according to the present invention is genistein (S. P. Verma et al., “Curcumin and Genistein, Plant Natural Products, Show Synergistic Inhibitory Effects on the Growth of Human Breast Cancer MCF-7 Cells Induced by Estrogenic Pesticides,” Biochem. Biophys. Res. Commun. 233: 692-696 (1997)). Genistein is a natural product found in soybeans. Genistein can be administered orally or by other routes.

K. Soybean Derived Lectins

Another class of additional compounds that can be used in methods according to the present invention is soybean derived lectins such as soybean agglutinin (S. Terashima et al., “Soybean Agglutinin Binding as a Useful Prognostic Indicator in Stomach Cancer,” Surg. Today 27: 293-297 (1997)). Soybean agglutinin can be administered orally and by other routes. Results show that these lectins may block bacteria-induced inflammation by binding to glycoprotein moieties which serve as receptors for activating bacterial superantigens (M. C. Y. Heng, unpublished data, 1998).

L. Dehydrozingerone

Another class of additional compounds that can be used in methods according to the present invention is the antioxidant dehydrozingerone and derivatives of dehydrozingerone (D. V. Rajakumar & M. N. Rao, “Antioxidant Properties of Dehydrozingerone and Curcumin in Rat Brain Homogenates,” Mol. Cell. Biochem. 140: 73-79 (1994).

The dosages and routes of administration of these additional compounds can be determined by the treating physician depending on the severity of the disease, the response to therapy, and other underlying medical conditions that are present.

IV. Pharmaceutical Compositions for Combined Therapy

Yet another aspect of the present invention is pharmaceutical compositions for combined therapy. These pharmaceutical compositions contain curcumin, a curcumin derivative or a curcuminoid that is in a solution containing one together with one or more of the following active agents:

(1) vitamin D₃ or vitamin D₃ analogues such as calcipotriene;

(2) vitamin A or vitamin A derivatives or analogues such as β-carotene or retinoids;

(3) calmodulin inhibitors such as zinc, cyclosporin A, anthralin, or trifluoroperazine;

(4) other anti-inflammatory drugs such as corticosteroids, substance P inhibitors such as capsaicin, resiniferatoxin, or capsaicin analogues, capsaicin-sensitive vanilloid receptor inhibitors such as capsazepine, cyclo-oxygenase inhibitors such as acetylsalicylic acid, and other non-steroidal anti-inflammatory agents such as naproxen;

(5) calcium channel blockers such as diltiazem;

(6) H1 and H2 histamine blockers;

(7) vitamin E (α-tocopherol) and other antioxidants and free radical scavengers such as reduced glutathione, β-carotene, catalase, and superoxide dismutase;

(8) polyphenolic compounds such as rutin, catechin, epicatechin, naringin, naringenin, gallotanin, and epigallotanin;

(9) monoterpenes;

(10) genistein;

(11) soybean derived lectins such as soybean agglutinin; and

(12) dehydrozingerone and its derivatives.

The compositions further comprise a pharmaceutically acceptable carrier.

In the composition, the curcumin, curcuminoid, or curcumin derivative is present in a quantity sufficient to detectably inhibit the activity of phosphorylase kinase in the blood of the mammal or in a tissue of the mammal to which the composition is administered as measured by phosphorylation of a suitable substrate, such as phosphorylase.

These compositions are useful for administration of two or more of these agents at the same time by the same route. Typically, the route is oral or topical, but can also be parenteral.

The other compound or compounds in the composition are present in a physiologically active quantity.

The dosage of each of the two or more pharmaceutically active agents in the combined pharmaceutical composition can be adjusted to meet clinical requirements and dosage ranges as described above.

A pharmaceutical composition for combined therapy according to the present invention is preferably in liquid or gel form, as discussed above for dosage forms for the individual therapeutic agents. However, other forms are possible. The curcumin, curcumin derivative or curcuminoid is present in a solution containing at least one alcohol, as detailed above.

The curcumin, curcumin derivative, or curcuminoid in the pharmaceutical composition can be in the form of a boron complex. The boron complex can be one of:

(1) a difluoroboron complex;

(2) a mixed complex in which the two fluorine atoms of a difluoroboron complex are replaced with the carboxyl oxygens of oxalic acid;

(3) a mixed complex in which the two fluorine atoms of a difluoroboron complex are replaced with a carboxyl group and a hydroxyl group of citric acid;

(4) a mixed complex in which the two fluorine atoms of a difluoroboron complex are replaced with the two hydroxyl groups of dibenzyl tartramide; and

(5) a mixed complex in which the two fluorine atoms of a difluoroboron complex are replaced with a second molecule of curcumin, the curcumin derivative, or the curcuminoid.

In a pharmaceutical composition according to the present invention, the curcumin, curcumin derivative, or curcuminoid can be present in a liposome. When the curcumin, curcumin derivative, or curcuminoid is present in a liposome, the preparation can be a preparation selected from the group consisting of a skin preparation, an eye drop preparation, a nasal drop preparation, an oral preparation, a pharyngeal preparation, a rectal preparation, a vaginal preparation, a bladder preparation, a urethral preparation, a parenteral preparation, and a bronchial preparation.

The invention is illustrated by the following Examples. These Examples are for illustrative purposes only and are not intended to limit the invention.

### EXAMPLE 1

### Solubility of Curcumin

Table 1 reports the results of a solubility study on curcumin. These results show that curcumin is not soluble in cold water or hot water. Curcumin is also not soluble in cold mineral oil or hot mineral oil. However, curcumin is soluble in 70% isopropyl alcohol to at least the extent of 0.5 g/50 ml.

TABLE 1
SOLUBILITY OF CURCUMIN

| Solvent | Volume | Result^{a} |
|---|---|---|
| Cold water | 5 ml | Not soluble (precipitate present) |
| Cold water | 50 ml | Not soluble (precipitate present) |
| Cold water | 500 ml | Not soluble (precipitate present) |
| Water (200° F.) | 5 ml | Not soluble (precipitate present) |
| Water (200° F.) | 50 ml | Not soluble (precipitate present) |
| Water (200° F.) | 500 ml | Not soluble (precipitate present) |
| Mineral oil | 5 ml | Not soluble (precipitate present) |
| Mineral oil | 50 ml | Not soluble (precipitate present) |
| Mineral oil (140° F.) | 5 ml | Not soluble (precipitate present) |
| Mineral oil (140° F.) | 50 ml | Not soluble (precipitate present) |
| 70% isopropyl alcohol | 5 ml | Soluble (some precipitate? |
| | | impurities) |
| 70% isopropyl alcohol | 50 ml | Soluble (no precipitate) |

^{a}0.5 g of curcumin used per experiment

### EXAMPLE 2

### Relative Effectiveness of Curcumin in Alcohol-based Preparations and in Oil-based Preparations

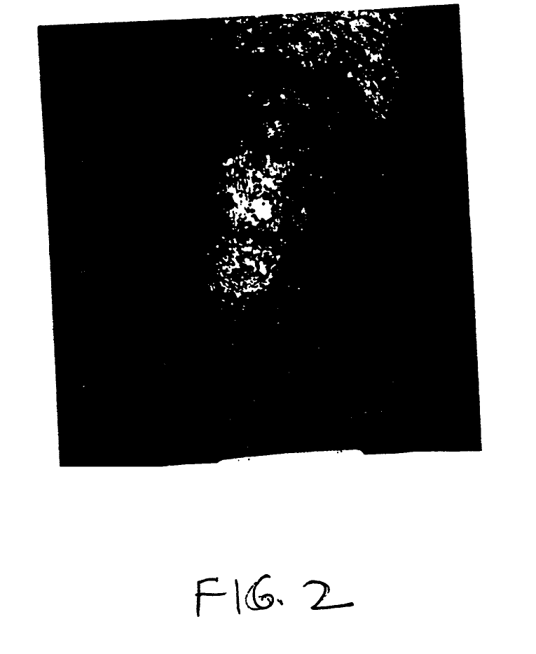

A study was performed to determine the relative effectiveness of curcumin in alcohol-based preparations and in oil-based preparations. Curcumin dissolved in a petrolatum or oil base was not effective in treating eczema and psoriasis (see FIG. 2).

To confirm this ineffectiveness of curcumin in a petrolatum or oil base in biochemical terms, the effect of curcumin in a petrolatum (oil) base and curcumin in a gel (alcoholic) base on phosphorylase kinase activity levels in psoriatic skin was determined. The results from two biopsies were compared, one before and one after treatment. The results are summarized in Table 2. Curcumin in an alcoholic gel base decreased phosphorylase kinase activity from 1254±473 units/mg protein before treatment to 198.5±91.5 units/mg protein after treatment. This difference was highly significant (p<0.010). On the other hand, curcumin in a petrolatum base failed to decrease phosphorylase kinase levels in psoriatic skin; phosphorylase kinase levels of pretreated skin (367±210 units/mg protein) were not decreased in treated skin, which showed phosphorylase kinase levels of 694±430 units/mg protein (p>0.05); the differences were not significant.

In conclusion, for curcumin to be effective as a phosphorylase kinase inhibitor, it must be in a soluble form; i.e., it must be dissolved in an alcohol or an alcoholic base such as a gel.

TABLE 2
INHIBITION OF PHOSPHORYLASE KINASE BY
CURCUMIN IN ALCOHOLIC GEL BUT NOT BY
CURCUMIN IN OIL BASE

| | Phosphorylase kinase | |
|---|---|---|
| | activity (units/mg protein) | |
| | Before curcumin | After curcumin |
| Curcumin in alcohol/gel base | 1254 ± 473 | 198.5 ± 91.5 |
| (n = 10) | | |
| Curcumin in petrolatum/oil | 367 ± 210 | 694 ± 430 |
| base (n = 6) | | |

### EXAMPLE 3

### Increase of Phosphorylase Kinase Activity in Dermatologic and Non-dermatologic Conditions

Phosphorylase kinase activity was increased in a large variety of dermatologic and non-dermatologic conditions, including inflammatory conditions. The results are shown in Table 3.

The results were obtained by the following methods:

Cytosolic Preparation of Epidermal Cells.

The biopsy samples were placed in a glass tube to which 3 ml Tris-HCl buffer (10 mM Tris-HCl, pH 7.8, 1 mM dithiothreitol (DTT), 3 mM MgSO₄ and 1 mM EGTA) was added, and homogenized vigorously with a Teflon plunger in a Tris-R model K41 homogenizer for 1 sec. Homogenization was repeated if necessary. The lysate of tissue cells was in the cytosolic solution. The lysates were centrifuged at 3,000×g for 15 min. Membranes and other cytosolic organelles which formed a pellet at the bottom of the tube were removed. The supernatant, which contained the cytosolic component of tissue cells, was then subjected to biochemical analysis.

Assay of Phosphorylase Kinase Activity.

Phosphorylase kinase (PK) activity was assayed by measuring the conversion rate of phosphorylase-b to phosphorylase-a according to a modification of the method of Cohen (P. Cohen, “Phosphorylase Kinase from Rabbit Skeletal Muscle,” Meth. Enzymol. 99: 243-250 (1983) and previously used (M. C. Y. Heng et al., “Elevated Phosphorylase Kinase Activity in Psoriatic Epidermis: Correlation with Increased Phosphorylation and Psoriatic Activity,” Br. J. Dermatol. 130: 298-306 (1994)).

Briefly, PK was assayed by measuring radioactive phosphate transferred from [³²P]ATP (DuPont Co., Wilmington, Del., USA) to the phosphorylase-b, suspended in 30 mM cysteine solution, pH 7.0, in the process of conversion to the phosphorylase-a form. Forty microliters of 30 mM cysteine solution, 50 μl of 0.25 M β-glycerophosphate solution, 50 μl of phosphorylase-b solution, and 20 μl of either standard solution or cytosolic samples were transferred to each 5.0 ml polypropylene test tube. The tubes were incubated at 30° C. for 3-5 min to equilibrate temperatures. At 0 min, 40 μl of [³²P]ATP solution was added to each tube, mixed thoroughly by vortexing, and incubated at 30° C. for 15 minutes. One milliliter of ice-cold trichloroacetic acid (TCA) solution was then added to each tube. The tubes were then placed in ice, and cooled for 10 min or more. Each reaction mixture was then filtered through a Millipore filter paper (pore size 0.45 μm) and washed three times with 2 ml of cold 5% TCA solution. The filter paper containing the phosphorylase-b was counted in a liquid scintillation counter. Enzyme activity was determined based on a standard curve prepared with phosphorylase kinase of known activity supplied by Sigma Co. (St. Louis, Mo., USA).

TABLE 3
PHOSPHORYLASE KINASE ACTIVITY IN NORMAL SKIN
AND MISCELLANEOUS DERMATOLOGICAL AND
NON-SKIN INFLAMMATORY CONDITIONS

| Condition | PK Activity (Units/mg protein) |
|---|---|
| Normal conditions | 7.2 |
| | 1.8 |
| | 3.1 |
| | 0.43 |
| | 1.51 |
| | 4.49 |
| Psoriasis | 2214.6 |
| | 182.1 |
| | 182.8 |
| | 146.3 |
| | 187.9 |
| | 208.8 |
| Acne | 14.9 |
| | 17.7 |
| Infected Eczema | 84.4 |
| | 13.1 |
| | 74.1 |
| | 50.5 |
| Sunburn | 17.64 |
| | 17.6 |
| Sun-damaged Skin (Premature Aging) | 11.29 |
| | 32.86 |
| | 19.88 |
| Skin Cancers | |
| Miscellaneous Inflammatory | |
| Conditions: | |
| Gastritis | 6.49 |
| Arthritis | 14.85 |
| AIDS (viral infection) | 110.4 |
| Wart (viral infection) | 8.06 |
| Lichenoid drug eruption | 7.64 |
| Esophagitis | 8.1 |
| Ulcerative Colitis | 2077.0 |

### EXAMPLE 4

### Increase of Phosphorylase Kinase Activity Following Traumatic Stimulus to Skin

Table 4 gives results showing that phosphorylase kinase activity can be elevated by a traumatic stimulus, such as tape-stripping. Elevations of phosphorylase kinase activity were observed in all tissues studied as early as 1 minute following injury.

The method for inducing trauma is the method of tape-stripping using repeated applications of tape to the skin to lift off the superficial layers of the skin. This was carried out in the skin of both quiescent psoriatic patients and patients without skin diseases. Biopsies of the normal/undamaged skin (controls) and tape-stripped skin (biopsied 1 min following tape-stripping) were assayed for phosphorylase kinase activity, using the assay as described in Example 3.

The results are shown in Table 4.

TABLE 4
PHOSPHORYLASE KINASE ACTIVITY IN
UNDAMAGED AND INJURED (TAPE-STRIPPED)
SKIN OF PSORIATIC AND NORMAL CONTROLS

| | Phosphorylase kinase activity (units/mg protein) | | |
|---|---|---|---|
| | Undamaged | Tape-Stripped | |
| | Skin | Skin | p Value |
| Psoriatic | 263 I 88.2 (n = 3) | 1489.3 I 1037.6 (n = 6) | p < 0.05 |
| Patients (qui- | | | |
| escent | | | |
| disease) | | | |
| Patients with | 38.1 I 12.5 (n = 4) | 1209 I 694 (n = 6) | p < 0.1 |
| no skin | | | |
| disease | | | |

### EXAMPLE 5

### Hsp60-Associated γδ T-cell Activation Precedes Smooth Muscle Cell Migration in Injured Arteries

Atherogenesis is currently believed to be an inflammatory response to acute or chronic endothelial injury (R. Ross, “The Pathogenesis of Atherogenesis: A Perspective for the 1990's,” Nature 362: 801-809 (1993)). The molecular and cellular mechanisms in atherogenesis are consistent with intimate involvement of cellular immune mechanisms in the inflammatory process (R. Ross (1993), supra; P. Libby & G. K. Hansson, “Biology of Disease: Involvement of the Immune System in Human Atherogenesis: Current Knowledge and Unanswered Questions,” Lab. Invest. 64: 5-15 (1991)). While it is known that the two most important cell types associated with cellular immunity, i.e. T-cells and macrophages, are ubiquitously present in atherosclerotic plaques (R. Ross (1993), supra; P. Libby & G. K. Hansson (1991), supra; J. M. Munro et al., “An Immunohistochemical Analysis of Human Aortic Fatty Streaks,” Human Pathol. 18: 375-380 (1987); E. E. Emerson & A. L. Robertson, Jr., “T Lymphocytes in Aortic and Coronary Intimas: Their Potential Role in Atherogenesis,” Am. J. Pathol. 130: 369-376 (1988); A. C. van der Wal et al., “Atherosclerotic Lesions in Humans: In Situ Immunophenotypic Analysis Suggesting an Immune Mediated Response,” Lab. Invest. 61: 166-170 (1989); G. K. Hansson & J. Holm, “Detection of Activated T Lymphocytes in the Human Atherosclerotic Plaque,” Am. J. Pathol. 135: 169-175 (1989)), the role of T-cells in the development of atherogenesis remains unclear. Among the unanswered questions regarding the role of T-cells are (a) whether an antigen-dependent T-cell response is responsible, in part, for starting the inflammatory process in atherogenesis; (b) if such a reaction does occur, which of the two known lineages of T-cells is primarily involved; (c) what is the antigen recognized by this T cell lineage; and (d) what is the early sequential relationship between the inflammatory response and the intimal migration of SMCs.

T-cells bearing the α/β− and γ/δ− T-cell antigen receptor (TCR) have been identified among the lymphocyte population of atherosclerotic plaques (R. Kleindienst et al., “Immunology of Atherosclerosis: Demonstration of Heat Shock Protein 60 Expression and T Lymphocytes Bearing α/β or γ/δ Receptor in Human Atherosclerotic Lesions,” Am. J. Pathol. 142: 1927-1937 (1993)). While the roles of the two T-cell lineages with respect to the questions raised above have not been fully clarified, several recent studies have suggested that γ/δ T-cells may play a pivotal role in atherogenesis, especially in the early stages of the inflammatory process. T-cells bearing TCR-γ/δ are found in early atherosclerotic lesions in densities which exceed their numbers in more mature plaques (R. Kleindienst et al. (1993), supra). Previous work has shown that the activated γ/δ T-cell is the earliest inflammatory cell detected in the adventitia and intima after arterial ligation injury in human arteries (M. K. Heng & M. C. Y. Heng, “Heat Shock Protein 65 and Activated γ/δ T-Cells in Injured Arteries,” Lancet 344: 921-923 (1994); M. C. Y. Heng et al., “Early Infiltration of Arterial Intima by Activated Dendritic γ/δ T Cells in Ligated Human Arteries: An Ultrastructural and Immunocytochemical Study,” Int. J. Angiol. 6: 167-172 (1997)). This T-cell was found within 4 hrs of arterial ligation and preceded the infiltration of macrophages and α/β T-cells into the site of injury (M. C. Y. Heng et al. (1997), supra).

The involvement of γ/δ T-cells in early atherosclerotic lesions is consistent with current concepts of their function in immune responses after tissue injury. While still the subject of debate, γ/δ T-cells appear to function as a first line of defense in the host inflammatory response to tissue injury (J. A. Bluestone et al., “TCR gamma/delta cells: A Specialized T-Cell Subset in the Immune System,” Ann. Rev. Cell Dev. Biol. 11: 307-353 (1995)), with the ability to respond within hours of the injury stimulus (M. K. Heng & M. C. Y. Heng (1994), supra; M. C. Y. Heng et al. (1997), supra; J. A. Bluestone et al. (1995), supra). This contrasts with the lag period of 3-7 days that occurs between antigenic stimulation and observed clonal expansion exhibited by most α/β T-cells (J. A. Bluestone et al. (1995), supra). The lag period of 3-7 days is also observed for α/β T-cell infiltration into injured sites after vascular and non-vascular injury (R. Ross & E. P. Benditt, “Wound Healing and Collagen Formation. 1. Sequential Changes in Components of Guinea Pig Skin Wounds Observed in the Electron Microscope,” J. Biophysiol. Biochem. Cytol. 11:677-700 (1961); R. S. Fishel et al., “Lymphocyte Participation in Wound Healing: Morphologic Assessment Using Monoclonal Antibodies,” Ann. Surg. 206:25-29 (1987)). The hypothesis that γ/δ T-cells are involved in the early events of atherogenesis is further supported by observations that γ/δ T-cells recognize stress proteins, specifically Hsp65 (M. C. Y. Heng et al. (1997), supra; A. Haregewoin et al., “Human Gamma/Delta T Cells Respond to Mycobacterial He at Shock Protein,” Nature 340:309-312 (1989); R. L. O'Brien et al., “Stimulation of a Major Subset of Lymphocytes Expressing T Cell Receptor Gamma/Delta by an Antigen Derived from Mycobacterium tuberculosis,” Cell 57:668-674 (1989)). An immune response mounted by γ/δ T-cells against Hsp65 expressed by the injured arterial wall may account for the early activation of this subset after arterial injury (M. K. Heng & M. C. Y. Heng (1994), supra; M. C. Y. Heng et al. (1997), supra). Of further interest in this regard is that experimental atherosclerosis has been produced by immunization of rabbits with Hsp65, and this is associated with infiltration of γ/δ T-cells in atherosclerotic lesions (Q. Xu et al., “Induction of Arteriosclerosis in Normocholesterolemic Rabbits by Immunization with Heat Shock Protein 65,” Arterioscler. Throm. 12: 789-799 (1992); Q. Xu et al., “Increased Expression of Heat Shock Protein 65 Coincides with a Population of Infiltrating T Lymphocytes in Atherosclerotic Lesions of Rabbits Specifically Responding to Heat Shock Protein 65,” J. Clin. Invest. 91:2693-2702 (1993)).

The question as to whether a T-cell/antigen reaction occurs in atherogenesis is not clear from review of the current literature. In atherosclerotic plaques, activated T-cells bearing both TCR lineages have been found among the inflammatory cell population of the lesions (R. Kleindienst et al. (1993), supra). Patterns of TCR gene utilization may be helpful in determining whether T-cells are activated by antigen-dependent or cytokine-dependent mechanisms. Thus, antigen-stimulated clonal expansion of α/β T-cells produces specific monoclonal patterns of Vβ gene utilization. An example is the demonstration of Vβ9 clonal expansion with T-cell stimulated by staphylococcal antigens (J. Kappler et al., “V Beta Stimulation of Human T Cells by Staphylococcal Toxins,” Science 244:811-813 (1989); Y. W. Choi et al., “Residues of the Variable Region of the T-Cell Receptor Beta-Chain that Interact with S. aureus Toxin Superantigens,” Nature 346:471-473 (1990)). By contrast, cytokine activation of α/β T-cells produces polyclonal patterns of Vβ gene utilization. In advanced human atherosclerotic lesions, the Vβ TCR gene utilization show a polyclonal pattern, with utilization of the 16 of 18 Vβ gene sequences, suggesting that activation of the α/β T-cells is probably cytokine rather than antigen dependent (S. J. Swanson et al., “Diversity of T-Cell Antigen Receptor Vβ Gene Utilization in Advanced Human Atheroma,” Arterioscler. Thromb. 14:1210-1214 (1994)). In an earlier study, α/β T-cells in human carotid plaques were reported to show total heterogeneity of the TCR genetic rearrangement, again suggesting a polyclonal origin and cytokine expansion of the αβ T-cell population (S. Stemme et al., “Polyclonal Origin of T Lymphocytes in Human Atherosclerotic Plaques,” Lab. Invest. 65: 654-660 (1991)).

The primary goal of the work reported in this Example was to test the hypothesis that a cellular immune reaction involving γ/δ T-cells occurs soon after induced arterial injury, that Hsp60 expressed by the arterial wall is the putative antigen recognized by this T-cell subset, and that the resulting immunological response, which includes activation of macrophages and α/β T-cells, is connected to the migration of smooth muscle cells from the media into the intima leading to intimal thickening.

Materials and Methods

(a) Rat Carotid Model for Hsp65 Production

In male Sprague-Dawley rats, arterial injury was produced by transient ligation of the carotid artery. The animals were anesthetized with Nembutal 40 mg/kg, and the right common carotid artery exposed through a mid-line incision. The lumen of the right common carotid artery was occluded with a “0” silk ligature for 15 mins, after which the suture was loosened sufficiently to reestablish blood flow, and left in place to mark the site of arterial ligation. A curved hemostat was placed between the ligature and the posterior wall of the artery. This minimized the pressure exerted by the ligature so that crush injury was minimized, while at the same time sufficient to completely occlude the lumen. In the sham-operated controls, the animals received only the mid-line skin incision, which was closed with sutures without an attempt to isolate and ligate the carotid vessels.

(b) Animals and Arterial Specimens

Fifty four animals, weighing about 600-800 gm each, were divided into the following groups: 36 in the ligated group and 18 in the sham-operated control group. In the ligated group, the animals (n=6 in each ligated subgroup) were sacrificed at 1, 4, 24, 48, 72 hrs and 3 months after carotid arterial ligation. Sham-operated controls (n=3 in each sub-group) were treated in the same manner. The part of the carotid artery 1 mm proximal to the ligated site to 1 cm distal of the ligature was processed for immunohistochemistry, electron microscopy and immunoelectron microscopy. Arterial specimens harvested 3 months after arterial ligation were processed only for light microscopy to determine extent of intimal thickening since we were interested mainly in the early inflammatory events.

(c) Immunohistochemistry

Arterial specimens were embedded in OCT compound (Tissue-Tek), snap-frozen and stored at −70° C. 4 μm sections were using a Tissue Tek II cryostat, mounted on poly-L-lysine-coated slides, air-dried at room temperature for 2 hrs to overnight, and air-dried after fixing in acetone at 4° C. Standard immunohistochemical techniques were carried out using the monoclonal antibodies listed in Table 5.

TABLE 5
MONOCLONAL ANTIBODIES DIRECTED AGAINST
RAT ANTIGENS FOR EXAMPLE 7

| | CLUSTER | | |
|---|---|---|---|
| | DESIGNATION/ | | |
| ANTIGENIC | CLONE/ | | |
| SPECIFIC | ISOTYPE | DILUTION | SOURCE |
| T lymphocytes | CD3/64.18/Ig63 | 1:100 | PharMingen |
| T lymphocytes | CD4/OX35/Ig62a | 1:200 | PharMingen |
| T lymphocytes | CD8a/0X8/IgG1 | 1:200 | PharMingen |
| Class II MHC | RTIB/OX6/IgG1 | 1:200 | PharMingen |
| IL-2 receptor | CD25/0X39/Ig62b | 1:200 | PharMingen |
| γ/δ T cells | TCR γ/δ/V65/IgG1 | 1:200 | PharMingen |
| α/β T cells | TCR α/β/R73/IgG1 | 1:200 | PharMingen |
| Hsp60 | LK-1/IgG1 | 1:200 | StressGen Biotech |
| Macrophages, | ED1/IC7/IgG1 | 1:100 | PharMingen |
| monocytes | | | |

TABLE 6
MORPHOMETRIC ANALYSIS OF LIGATED RAT
CAROTID ARTERIES FOR EXAMPLE 5

| SPECIMENS | 1 HR | 4 HR | 24 HR | 48 HR | 72 HR |
|---|---|---|---|---|---|
| Dendritic γ/δ T cells | 0-1 | 2.8 ± 0.8* | 9.0 ± 2.1** | 11.8 ± 2.5** | 18.9 ± 4.2** |
| (No./2 mm section) | | | | | |
| HSP-DTC interactions | 0-3 | 15.8 ± 2.8** | 65.5 ± 10.3** | 45.0 ± 6.4*** | 22.4 ± 35*** |
| (No./2 mm section) | | | | | |
| DTC-macrophage | 0 | 0-1 | 3.0 ± 1.4* | 11.8 ± 2.5* | 13.5 ± 2.4* |
| interactions (No./2 mm | | | | | |
| section) | | | | | |
| α/β T cells (No./hpf) | 0 | 0 | 0 | 0-1 | 5.6 ± 1.5* |
| Macrophages (No./hpf) | 0 | 0-1 | 3.3 ± 1.0 | 16.9 ± 4.5** | 32.7 ± 8.6** |
| Intimal SMC (No./2 mm | 0 | 0 | 0-1 | 2.8 ± 1.0* | 8.3 ± 2.5** |
| section) | | | | | |

HSP=heat shock protein, DTC=dendritic T cell, SMC=smooth muscle cell, hpf=high power field

N=6 per group per time point for ligated arteries and

N=3 per group per time point for sham-operated controls

*(p<0.05); **(p<0.01); ***(p<0.001) compared to controls

TCR γ/δ+ T cells, ED1+ macrophages, TCR α/β+ T cells, and intimal SMC were not consistently observed in control arteries

Slides were immunostained using DAKO LSAB2 kit as follows: (1) PBS wash (20 mins); (2) endogenous biotin block technique using DAKO Biotin Block system; (3) PBS wash (10 mins); (4) incubation with monoclonal antibody (Table 1) for 48 hours at 4° C.; (5) PBS wash (10 mins); (6) incubation with biotinylated anti-mouse immunoglobulin; (7) PBS wash (10 mins); (8) incubation with either streptavidin-conjugated peroxidase or strepavidin-conjugated phosphatase; (9) PBS wash (10 mins); (10) labeling with either diaminobenzidine or Fast Red (with levamisole to suppress endogenous alkaline phosphatase); (11) counterstaining with Mayer's hematoxylin.

(d) Electron-microscopy

Arterial specimens were fixed in glutaryldehyde, buffered to pH 7.3 with 0.1M sodium cacodylate, post-fixed in 1% osmium tetroxide, treated with tannic acid, dehydrated in alcohol and propylene oxide, embedded in a mixture of Epon 812 and Araldite 502. Sections (2×2 mm; 70-80 nm thick) were cut with an ultramicrotome with a diamond knife (Dupont), mounted onto 200 mesh grids, stained with lead acetate and examined under a Philips 201 electron microscope.

(e) Immunoelectron Microscopy

Immunoelectron microscopy using immunogold-labeled was carried out using a post-embedding method using streptavidin-labeled colloidal gold particles and a biotinylated second antibody in post-fixed specimens processed for electron microscopy as previously described (M. K. Heng & M. C. Y. Heng (1994), supra). Briefly, ultrathin sections were fixed in 2.5% glutaraldehyde, post-fixed with osmium tetroxide, and stained with tannic acid. They were mounted onto uncoated nickel grids and incubated with mouse monoclonal antibody to Hsp60 (LK-1/IgGl, StressGen Biotech) in dilutions of 1:200. Next, they were washed with PBS, incubated with biotinylated anti-mouse IgG, and washed again with PBS. The sections were then incubated with colloidal gold particles (5 nm) bound to streptavidin (1 in 5 dilution), washed with PBS, stained with uranyl acetate and lead citrate, and examined under a Philips EM201 electron microscope.

(f) Morphometry

The dendritic γ/δ T-cells, identified ultrastructurally by the presence of lymphoid nuclei with dense chromatin, long dendritic processes (P. R. Bergstresser et al., “Dendritic T Cells: Lesson from Mice for Humans,” J. Invest. Dermatol. 100: 80s-83s (1993); C. E. Grossi et al., “Human T Cell Expressing the γ/δ T-cell Receptor (TcR-1): C-γ1- and C-γ2-Encoded Forms of the Receptor Correlate with Distinctive Morphology, Cytoskeletal Organization and Growth Characteristics,” Proc. Natl. Acad. Sci. USA 86:1619-1623 (1989)) and dense cytoplasmic granules (H. Koizumi et al., “Expression of Perforin and Serine Esterases by Human Gamma/Delta T Cells,” J. Exp. Med. 173:499-502 (1991); M. Nakata et al., “Expression of Perforin and Cytolytic Potential of Human Blood Lymphocyte Subpopulations,” Int. Immunol. 4:1049-1054 (1992)), and confirmed by TCR-γ/δ positivity on immunohistochemical sections, were quantified as the number/2 mm section of arterial wall (mean of 10 interrupted sections examined per 2 mm block). Additionally, ten or more consecutive sections were used to determine the cell type when only dendritic processes were noted and the nucleus or cytoplasmic granules were not visualized. Consecutive sections were also used to determine the cells hidden behind the grid bars. Smooth muscle cells were identified ultrastructurally by the presence of characteristically fine 10K filaments and by the presence of basement membrane. The data obtained from each specimen constitutes the number of intimal SMCs/2 mm section. Hsp60-dendritic T-cell interactions and macrophage-lymphocyte interactions were quantified ultrastructurally as the number of interactions/2 mm section. Recruited α/β T-cells and macrophages were assessed in immunohistological sections by the presence of TCRα/β+ and ED1+ antigen (for monocytes and macrophages) respectively, and quantified as the number/2 mm section and number/hpf respectively.

(g) Statistical Analysis

Comparison of the differences between mean measurements of the different groups were by the two tailed Student's t test. The results were considered statistically significant when p<0.05. Results are expressed as mean (SD).

Results

To test the hypothesis that activation of γδ T-cells by autologous hsp60 is partly responsible for the inflammatory response in the injured arteries, the sequential appearance of hsp60, γ/δ T-cells, α/β T-cells, macrophages and SMC in the intima was noted. Because activated T-cells interact with macrophages, T-cell-macrophage interactions were also counted to determine which of the T-cell lineages was involved in early activation of the immune response. The number of cells and cellular interactions per section or high power field (hpf) are shown in Table 6.

(1) Sequence of Appearance and Identification of Hsp60 in Injured Rat Arteries

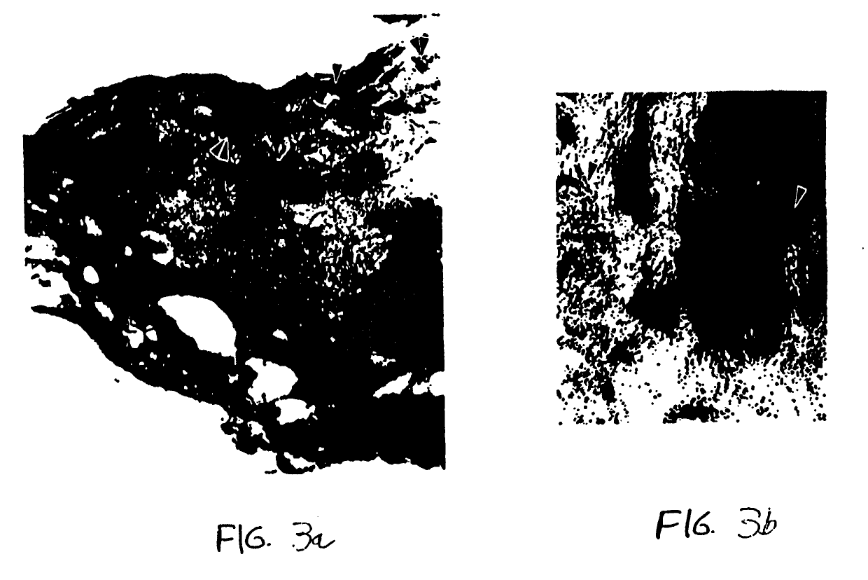

Expression of Hsp60 at the site of injury was the earliest change noted in ligated arteries. Hsp60 was detected at 1 hr post-ligation in ligated arteries but not in any of the sham-operated controls. Positive staining for Hsp60 was found by immunohistochemistry to be present both intracellularly (FIG. 3a) and to accumulate extracellularly as a fibrillary protein (FIG. 3a). The fibrillary morphology of the protein is best observed ultrastructurally with tannic acid staining (FIG. 3a, FIG. 3b). Immunogold labeling by immunoelectron microscopy identified this fibrillary protein tannic acid-stained protein as Hsp60 (FIG. 3b). Tannic acid staining was thus used as a marker to screen for the presence of hsp65 in subsequent ultrastructural sections.

(2) Infiltration of Ligated Arteries by Dendritic γδ T-cells

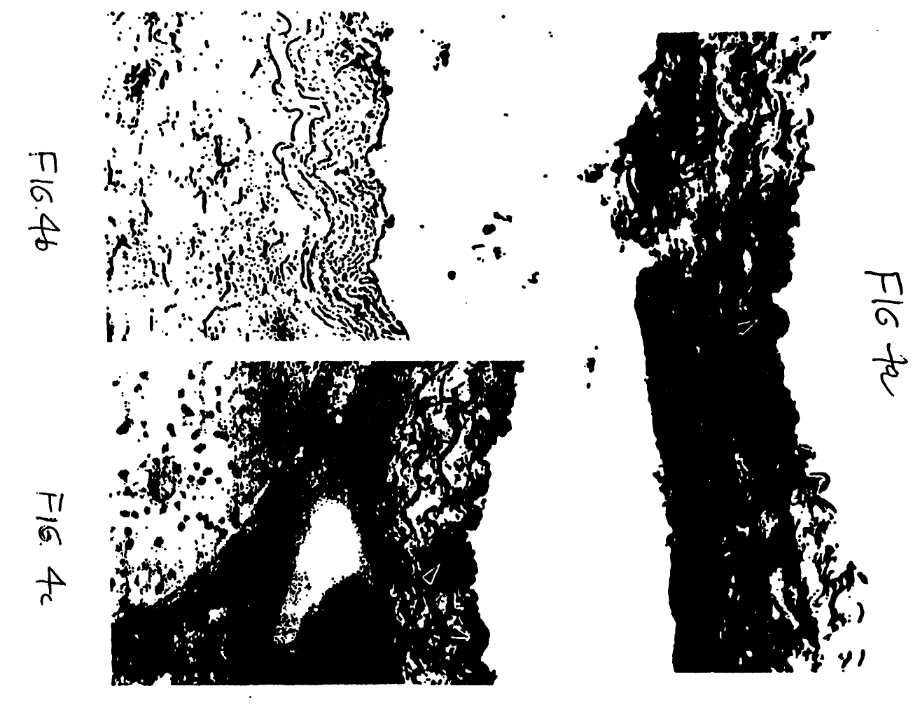

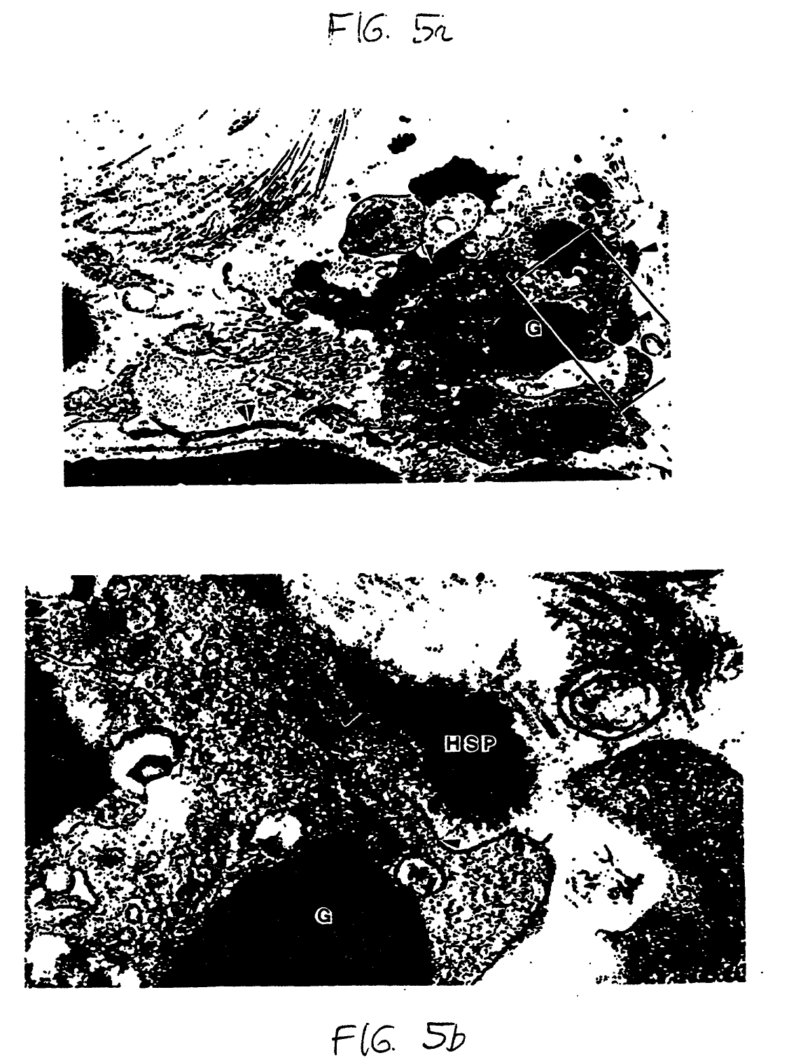

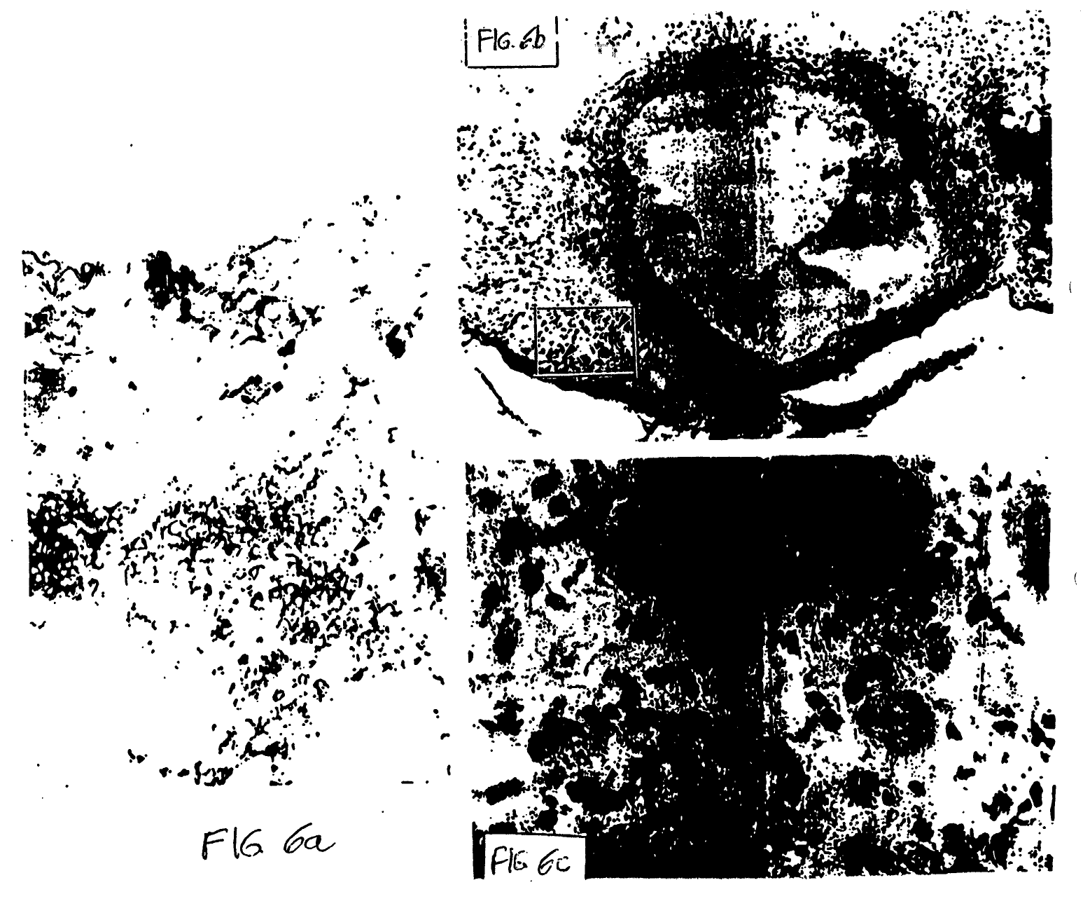

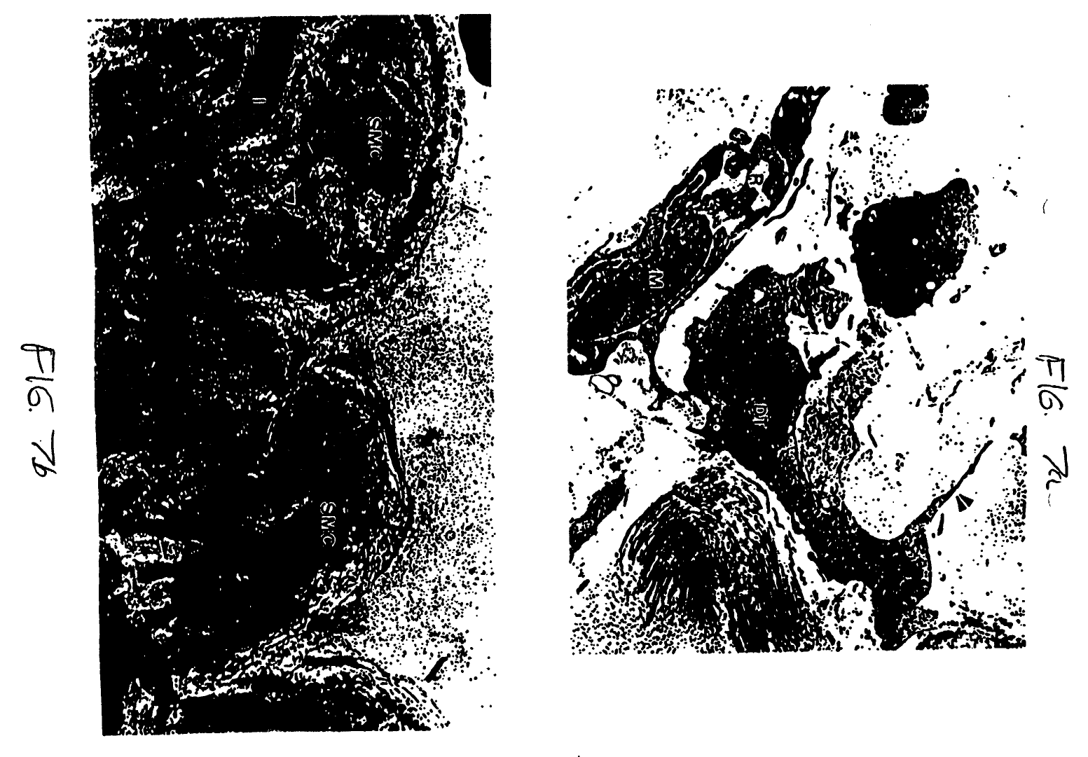

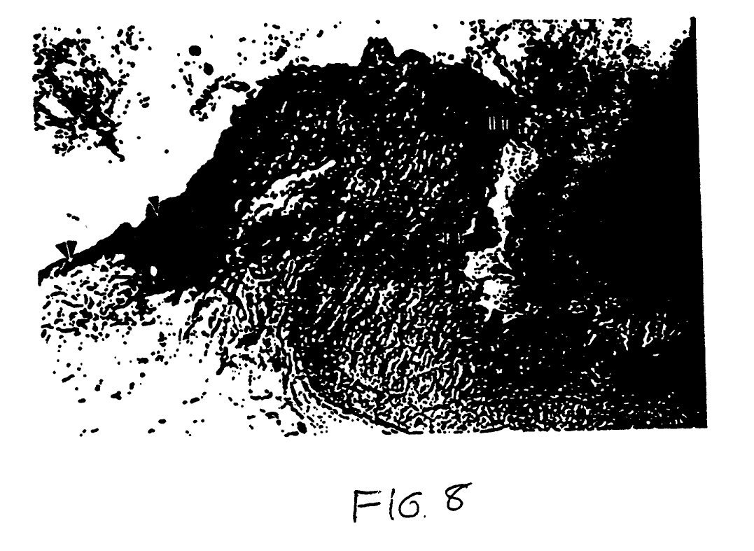

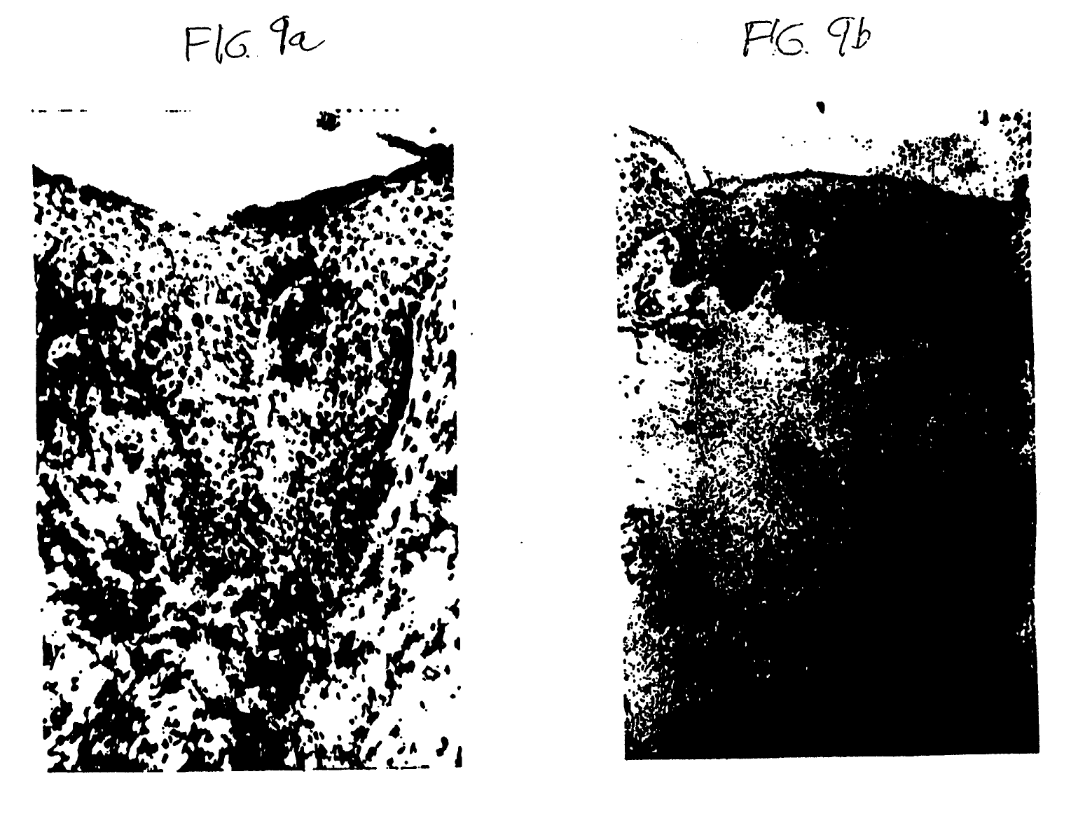

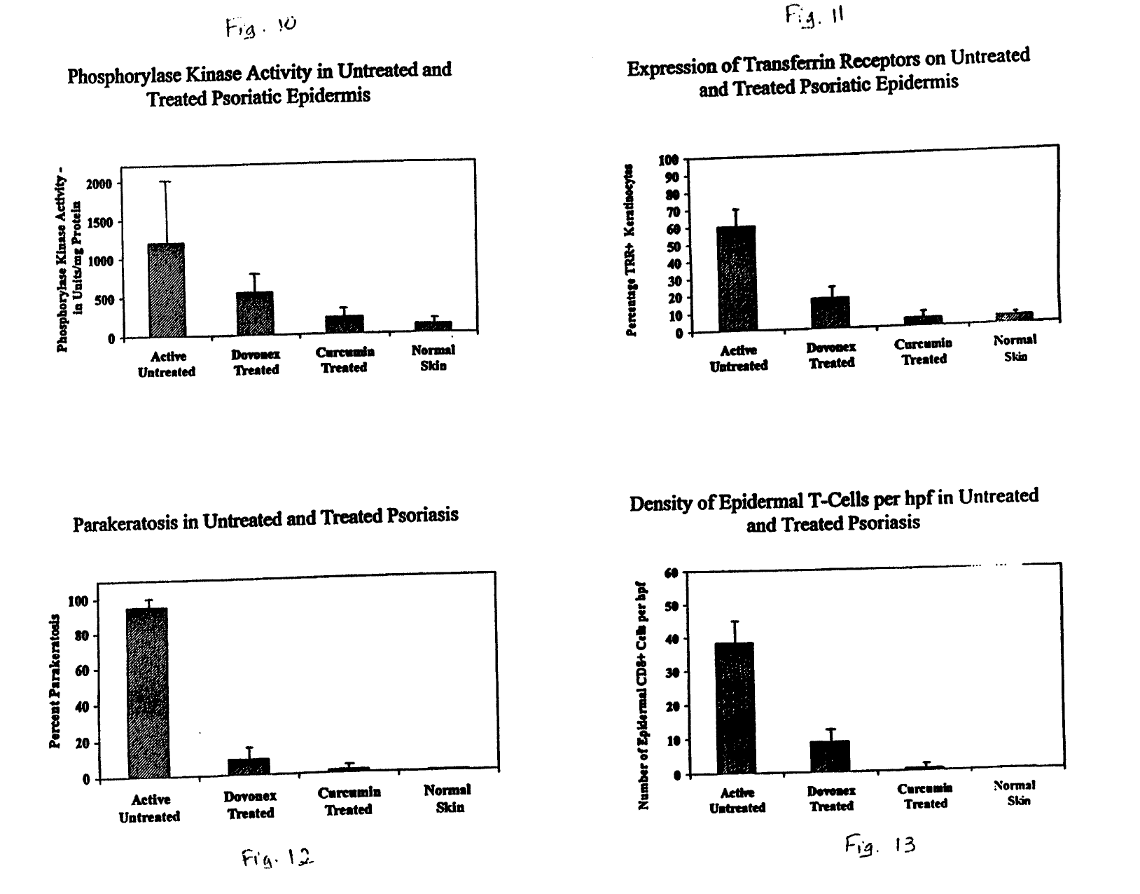

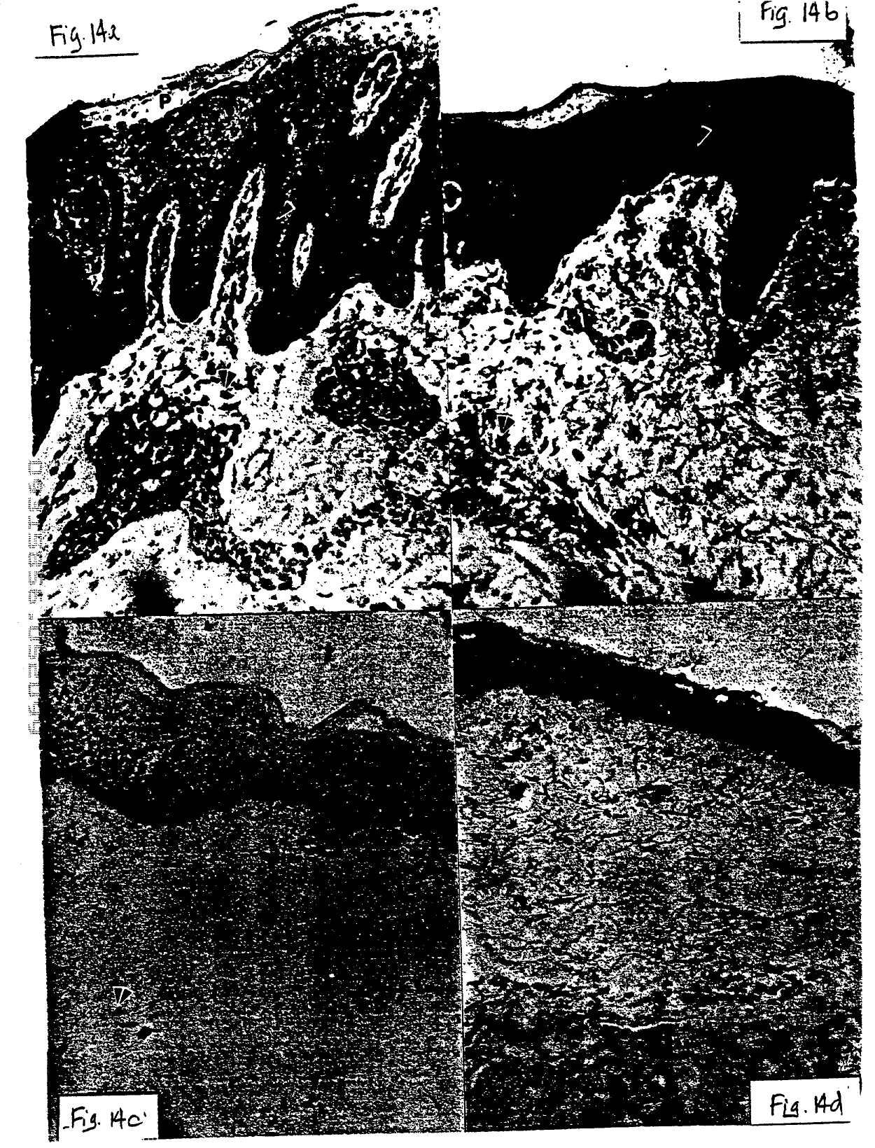

An infiltrate of TCRγ/δ+ T-cells in the intima and adventitia of ligated arteries was observed to be clearly present 4 hrs post-ligation by immunohistochemical techniques (FIG. 4a, Table 6). These cells are characterized by long dendritic processes (FIG. 4b, FIG. 5a), as well as by the presence of electron-dense intracytoplasmic perforin granules (FIG. 5a). The number of these cells increased over the 72 hours following arterial ligation (Table 6). γ/δ T-cells were not found in the sham-operated control arteries.

(3) Close Contact Between Tannic Acid-stained HsR60 and Dendritic γδ T-cells is Followed by Activation of Dendritic γδ T-cells in Ligated Arteries

Because surface contact between the antigen and the TCR (contact-interactions) is necessary for antigen recognition by the TCR, close apposition of Hsp60 to T-cells was used as a marker for T-cell-antigen interaction in the recognition process. Contact-interaction between tannic acid stained hsp60 and a dendritic subset of granular T-cells (FIGS. 5a, 5b) was clearly observed at 4 hrs post-ligation, peaked at 24 hrs and then declined (Table 6). The closeness of the contact points between the tannic acid-stained fibrillary Hsp60 and the cell surface of dendritic T-cells is shown in FIG. 5b. These dendritic T-cells were shown by immunohistochemical studies to be TCR-γ/δ+(FIG. 4a), CD3+(weak), CD4−, CD8− and TCRα/β−. The subsequent activated status of the dendritic T-cells was shown by their expression of activation markers such as MHC class II (RT1B; FIG. 4b) molecules, and IL-2R (CD25; FIG. 4c). These activation markers were detected as early as 4-24 hrs post ligation in the injured ligated arteries (FIG. 4b) but not in any of the sham-operated control specimens.

(4) Sequential Infiltration of αβ T-cells, Macrophages and Intimal SMCs

The presence of monocytes/macrophages was confirmed by immunohistochemical labeling using anti-ED1 monoclonal antibodies. Infiltration of macrophages into ligated arteries was clearly established at 24 hours, and their numbers increased over 72 hours (Table 6). While numerous in the adventitia at 72 hours (FIG. 6a), macrophages were rarely seen in the intima. Macrophages were absent or sparse (<1/hpf) at all time points in sham-operated controls.

Ultrastructurally, these macrophages were characterized by the presence of sparse nuclear chromatin pattern, and by the presence of well-developed rough endoplasmic reticulum (FIG. 7a). Macrophages were observed ultrastructurally to have surface contact (interactions) with dendritic T-cells (FIG. 7a). These macrophage-dendritic T-cell contact-interactions were sparse at 24 hours, but numerous at 72 hrs in the ligated arteries, but not seen in sham-operated controls.

The least numerous immunocompetent cell-type observed were lymphocytes of the α/β T-cell lineage. These cells were identified immunohistochemically by their TCRα/β positivity.

TCRα/β+ cell infiltration into ligated arteries occurred late, and were clearly established only at 72 hours (Table 6). They were mainly observed within the adventitia (FIGS. 6b, 6c), and occasionally at the luminal-endothelial junction but were not observed within the intima.

Vascular SMCs in the process of migrating from the media into the intima were observed in the 48- and 72-hr ligated arteries (FIG. 7b), but not seen in any of the control arteries. SMC migration was established at 48 hours (Table 6) and increased thereafter to result in intimal hyperproliferation at 3 months (FIG. 8). In the 3-month arterial specimens, focal intimal thickening (2.5-10×) was observed immediately distal to the site of arterial ligation (FIG. 8) in 6/6 ligated rats. Focal intimal thickening was not observed in controls at 3 months. A sharp demarcation between the normal intima and the thickened intima, separated by the site of arterial ligation (FIG. 8), was observed in the ligated specimens.

Discussion

γδ T-cells and Other Immunocompetent Cells in Smooth Muscle Cell Proliferation and Intimal Thickening

While the inflammatory process in atherogenesis is clearly associated with close involvement of immune competent cells i.e. T-cells and macrophages (R. Ross (1993), supra; P. Libby & G. K. Hansson (1991), supra; J. M. Munro et al. (1987), supra; E. E. Emerson & A. L. Robertson, Jr. (1988), supra; A. C. van der Wal (1989), supra; G. K. Hansson & J. Holm (1989), supra), the specific subset of T-cells involved in initiation of the immune response and the antigen they recognize have not been identified. The findings reported in this Example support the role of an immune-mediated inflammatory response being mechanistically related to intimal migration of smooth muscle cells at 48-72 hours and their subsequent proliferation, leading to intimal thickening at 3 months. The early intimal appearance of the γ/δ T-cells during the period when α/β T-cells were not detected suggests that it is activation of γ/δ T-cells by Hsp60 expressed by arterial injury that may be responsible for initiating the inflammatory response.

Hsp60 Expression is Caused by Arterial Injury

The findings of this study are consistent with the above hypothesis. Carotid artery injury in rats was followed by early Hsp60 expression (1 hr), and sequential infiltration of γ/δ T-cells (4 hrs), macrophages (24 hrs), and α/β T-cells (72 hrs). Of special interest is the appearance of the immunolabeled Hsp60 in the rat artery, which was remarkably similar to that reported earlier in human ligated arteries (M. K. Heng & M. C. Y. Heng (1994), supra; M. C. Y. Heng et al. (1997), supra). Expression of Hsp60 after carotid ligation in the model reported in this Example is a phenomenon seen in all in eukaryotic cells, which produce Hsp in response to a variety of tissue injury (R. A. Young & T. J. Elliot, “Stress Proteins, Infection and Immune Surveillance,” Cell 59:5-8 (1989)). What is especially significant in the context of atherogenesis, however, is a growing awareness that Hsps are the dominant antigens in immune responses to external and internal agents, e.g. infections and autoimmune diseases (R. A. Young & T. J. Elliot (1989), supra; T. M. Shinnick, “Heat Shock Proteins as Antigens of Bacterial and Parasitic Pathogens,” Curr. Top. Microbiol. Immunol. 167:145-160 (1991)). Recent evidence implicates Hsps as antigens involved in vascular immune reactions in atherogenesis. Immunization of normocholesterolemic rabbits with mycobacterial Hsp65 has been shown to induce experimental atherosclerosis (Q. Xu et al. (1992), supra; Q. Xu et al. (1993), supra). In humans, Hsp70 has been detected in atherosclerotic plaques (P. A. Berberian et al., “Immunohistochemical Localization of Heat Shock Protein-70 in Normal Appearing and Atherosclerotic Specimens of Human Arteries,” Am. J. Pathol. 136:71-80 (1990)), and titers of antibodies to Hsp60, the human homologue of mycobacterial Hsp65 (W. Jarjour et al., “Constitutive Expression of a Gro-EL-Related Protein on the Surface of Human γδ T-Cells,” J. Exp. Med. 172:1857-1860 (1990)), correlate with severity of carotid atherosclerosis (Q. Xu et al. (1993), supra). Accordingly, an immune response to Hsp60 produced after vascular injury may be responsible, at least in part, for the inflammatory response to atherosclerosis.

Presence of Activated γ/δ T-cells

It was also found that γ/δ T-cell activation and recruitment into the ligated arteries preceded that of other inflammatory cells, i.e., macrophages and α/β T-cells. γ/δ T-cells were detected by monoclonal antibodies specific for TCRγ/δ, and by ultrastructural features reported previously (P. R. Bergstresser et al. (1993), supra; C. E. Grossi et al. (1989), supra; H. Koizumi et al. (1991), supra; M. Nakata et al. (1992), supra). Contact interactions between γ/δ T-cells and Hsp60, with evidence of subsequent γ/δ T-cell activation indicate that γ/δ T-cells may recognize Hsp60 produced after arterial ligation in the model reported in this Example. There is clear evidence from previous studies that γ/δ T-cells react with focused specificity to Hsp60 (A. Haregewoin et al. (1989), supra; R. L. O'Brien et al. (1989), supra).

The rapidity with which activated γ/δ T-cells were detected in the ligated arteries in the study reported in this Example, i.e., within 4 hr, is consistent with current knowledge of the biology of this unique lymphocyte. The dendritic γ/δ T-cell is functionally and morphologically distinct from the α/β T-cell (J. A. Bluestone et al. (1995), supra; P. R. Bergstresser et al. (1993), supra; C. E. Grossi et al. (1989), supra; H. Koizumi et al. (1991), supra; M. Nakata et al. (1992), supra). Functionally, some γ/δ T-cell clones appear to recognize antigens differently from α/β T-cells, i.e. they are able to recognize antigens at the cell-surface without prior intracellular processing by an antigen presenting cell (Y. H. Chien et al., “Recognition by γ/δ T-Cells,” Annu. Rev. Immunol. 14:511-532 (1996); R. Sciammas et al., “Unique Antigen Recognition by a Herpesvirus-Specific TCRγ-δ cell,” J. Immunol. 153:3051-3058 (1994); B. C. Weintraub et al., “γ-δ T-Cells Can Recognize Non-Classical MHC in the Absence of Conventional Antigenic Peptides,” J. Immunol. 153:3051-3058 (1994)). This allows rapid activation of these T-cells, which may serve an important biologic function in rapid immune surveillance and defense. Bluestone suggests that because of the lag period of 3-7 days between antigenic stimulation and observed clonal expansion by α/β T-cells, γ/δ T-cells may function as the means employed by the immune system to mount an aggressive early immune response during periods of extreme stress (J. A. Bluestone et al. (1995), supra).

Morphologically, the γ/δ T-cells found in the study reported in this Example were characterized by long dendritic cytoplasmic processes of filopodia (P. R. Bergstresser et al. (1993), supra; C. E. Grossi et al. (1989), supra) and electron-dense intracytoplasmic perforin granules (H. Koizumi et al. (1991), supra; M. Nakata et al. (1992), supra). Monoclonal antibodies directed against TCRγ/δ have identified two non-overlapping subsets of γδ T-cells—a dendritic subset with long dendritic filopodia and enhanced spreading properties (C. E. Grossi et al. (1989), supra; FIGS. 4b, 5a), and a blood-borne subset in which the filopodia is much less marked (C. Bottino et al., “Two Subsets of Human Lymphocytes Expressing γ/δ Antigen Receptor Are Identifiable by Monoclonal Antibodies Directed to Two Distinct Molecular Forms of the Receptor,”. J. Exp. Med. 168:491-505 (1988)). The dendritic γ/δ T-cell may be a subpopulation particularly adapted for tissue infiltration. The electron dense cytoplasmic granules, which contain perforin and proteolytic enzymes, have been noted by others to be characteristic ultrastructural markers of γ/δ T-cells (H. Koizumi et al. (1991), supra; M. Nakata et al. (1992), supra).

Sequence of Cellular Interaction and Migration

The sequence of cellular activity after arterial injury suggests an inflammatory process initiated and amplified by Hsp60 activation of the γ/δ T-cell. On ultrastructural examination, numerous contact interactions were observed between the γ/δ T-cell dendritic processes with Hsp60 and macrophage in ligated arteries. Contact interactions between T-cells and its cognate antigen are believed essential to stimulation and activation of the lymphocyte (K. Inaba & R. K. Steinman, “Accessory Cell T-Lymphocyte Interactions. Antigen-Dependent and Independent Clustering,” J. Exp. Med. 163:247-261 (1986)). Interactions between γ/δ T-cells and Hsp60 were well established by 4 hrs post-ligation in the model reported in this Example. The observation that interactions between hsp60 and γ/δ T-cells were followed by rapid (within hours) MHC class H molecules and IL-2R expression by the lymphocytes suggests direct activation of γ/δ T-cells by Hsp60 without intracellular antigen processing (Y. H. Chien et al. (1996), supra; R. Sciammas et al. (1994), supra; B. C. Weintraub et al. (1994), supra). The number of these interactions peaked at 24 hrs and declined after this time, possibly due to diminishing Hsp production 24 hrs after arterial injury. The biologic processes that occurred during contact interactions between γ/δ T cells and macrophages are less clear. While some γ/δ T-cell clones are capable of recognizing unprocessed antigens without the requirement for MHC molecules (Y. H. Chien et al. (1996), supra; R. Sciammas et al. (1994), supra; B. C. Weintraub et al. (1994), supra), others have been shown to recognize processed antigens in the context of MHC molecules (H. Schild et al., “The Nature of Major Histocompatibility Complex Recognition by γδ T-Cells,” Cell 76:29-37 (1994)). It is, therefore, possible that the observed γ/δ T-cell-macrophage interaction represents recognition by TCRγ/δ of processed antigen by macrophages. This process, which may occur in addition to recognition by the TCRγ/δ of unprocessed antigen, may serve to further enhance the inflammatory response. In contrast, contact interaction was not observed between α/β T-cells with macrophages in ligated arteries, at least within the first 72 hours after arterial injury. This suggests that activation of the α/β T-cell subset, unlike the γ/δ T-cells, is probably not antigen-dependent in this setting. This hypothesis is consistent with previous studies that have concluded that activation of α/β T-cells in advanced atherosclerotic arteries appear to be cytokine rather than antigen-dependent (S. J. Swanson et al. (1994), supra; S. Stemme et al. (1991), supra).

In conclusion, the results in this Example showed that Hsp60 expression and γ/δ T-cell migration and activation was the earliest inflammatory activity noted after arterial ligation. This was followed by infiltration of macrophages, vascular SMCS, and α/β T-cells into the injured arterial wall. The density of all four cell types increased over the 72 hrs of study. This sequence of events suggests that a cellular immune response initiated by activation of migrated γ/δ T-cells by Hsp60 leads to amplification of the inflammatory response presumably through cytokines produced by immune-competent inflammatory cells.

### EXAMPLE 6

### Correlation of Phosphorylase Kinase Activity with Chararacteristic Markers of Psoriatic Activity

To test the hypothesis that increased phosphorylase kinase activity may underlie psoriatic activity, including activity related to the increased migratory activity of inflammatory cells into uninvolved psoriatic skin and also activity related to the increased migratory activity of keratinocytes, phosphorylase kinase activity was assayed in skin biopsies in a number of patients. Skin biopsies taken from 10 patients each with: (a) untreated/active psoriasis; (b) resolving psoriasis treated with curcumin; and (c) resolving psoriasis treated with the vitamin D₃ analogue Donovex. In all three groups, phosphorylase kinase levels were correlated with psoriatic activity as assessed by the following: (a) TRR+, a marker for DNA synthetic (cycling) keratinocyte population; (b) severity of parakeratosis, a marker of the migratory capacity of an immature keratinocyte population migrating from the basal layers to the stratum comeum; (c) CD8+ lymphocytes within the epidermis, to reflect T cells which have migrated from the bloodstream into the epidermis; and (d) HLA-DR expression, a marker of cytokine-activated (C. E. Griffiths et al., “Characterization of Intercellular Adhesion Molecule-1 and HLA-DR Expression in Normal and Inflamed Skin; Modulation by Recombinant Gamma Interferon and Tumor Necrosis Factor,” J. Am. Acad. Dermatol. 25: 778-786 (1991)) inflammatory and non-inflammatory cells.

Methods

(A) Participants:

With consent, punch biopsies were collected (6 mm) from active plaques of 10 patients with active untreated psoriasis, from resolving plaques from 10 patients each treated with curcumin alcoholic gel (1%) for 4-6 weeks, and with Dovonex ointment for 6-18 months. Normal skin was also biopsied from 10 non-psoriatic patients to monitor the results.

(B) Cytosolic Preparation of Epidermal Cells:

From each site, skin biopsy samples (6 mm punch) were stored at −70° C., and processed thereafter as previously described (Heng et al. (1994), supra). Briefly, each frozen sample, with the epidermal surface facing upwards, was placed in a glass tube to which 3 ml Tris-HCl buffer (10 mM Tris-HCl, pH 7.8, 1 mM dithiothreitol (DDT), 3 mM MgSO₄ and 1 mM EGTA) was added. This was homogenized vigorously with a Teflon plunger in a TRIS-R model K41 homogenizer for 1 sec. Homogenization was repeated if necessary. The lysate of epidermal cells, separated from the dermis, was in the cytosolic solution. The fibroblasts that were inadvertently detached, together with the relatively intact piece of dermis, were removed by decanting the cytosolic solution into a 5 ml capacity polypropylene test tube. The lysates were centrifuged at 3,000×g for 15 mins. Membranes and other cytosolic organelles which formed a pellet at the bottom were removed. The supernatants, which contained mainly the cytosolic component of epidermal cells, with some contaminant dermal cells, were then subjected to biochemical analysis.

(C) Assay of Phosphorylase Kinase Activity:

The serine kinase activity of phosphorylase kinase was assayed by measuring the incorporation of ³²P into phosphorylase-b according to a modification of the method of Cohen as previously described (Heng et al. (1994), supra). Briefly, phosphorylase kinase was assayed by measuring radioactive phosphaste transferred from [³²P]ATP (Dupont Co, Wilmington, Del.) to phosphorylase-b, suspended in 30 mM cysteine solution, pH 7.0, in the process of conversion to the phosphorylase-a. To each 5.0 ml polypropylene test tube, 40 T1 of 30 mM cysteine solution, 50 T1 of 0.25 M l-glycerophosphate solution, 50 T1 phosphorylase-b solution, and 20 T1 of either phosphorylase kinase standard solution or cytosolic samples were transferred. The tubes were incubated at 30° C. exactly for 15 mins. Then, 1.0 ml of ice cold 5% trichloracetic acid (TCA) solution was added to each tube. The tubes were placed in ice and cooled for 10 mins or more. Each reaction mixture was then filtered through a Millipore filter paper (pore size 0.45 Tm), and washed 3 times with 2 ml of cold 5% TCA solution. The filter paper containing phosphorylase-b was counted in a liquid scintillation counter. Enzyme activity was determined based on the standard curve prepared with phosphorylase kinase of known activity supplied by Sigma Co (St. Louis, Mo.).

(D) Immunocytochemistry:

Biopsy specimens for immunocytochemistry were embedded in OCT compound (Tissue-Tek), snap-frozen in liquid nitrogen and stored at −70° C. Serial cryostat sections (4 Tm) were mounted on gelatin-coated slides and air-dried for 30 mins at room temperature. The mounted sections were then freeze-dried for 4 hours, fixed in acetone for 20 mins at room temperature, and air-dried for 5 mins prior to immunostaining. Monoclonal antibodies (as listed at Table 7), were added to the sections, which were then incubated for 60 mins at room temperature, washed in phosphate-buffered saline (PBS) pH 7.6, and overlaid with peroxidase conjugate. All monoclonal antibodies had been obtained from Becton-Dickinson and were used at 1:100 dilution. The sections were then washed with PBS and the peroxidase color developed using diaminobenzidine. Negative controls from all patients were processed as above, omitting the monoclonal antibodies. Further controls were stained with kappa chain monoclonal antibodies and cytokeratin to monitor the procedure.

TABLE 7
MONOCLONAL ANTIBODIES USED IN EXAMPLE 6

| | Clone/Cluster | |
|---|---|---|
| Antibody | Designation | Specificity |
| Leu 2a | SK1/mouse IgG1/CD8 | Cytotoxicity/suppressor T |
| | | cells, certain NK cell subsets |
| Leu 3a | SK3/mouse IgG1/CD4 | Helper/inducer T cells, |
| | | activated macrophages |
| Leu 4 | SK7/mouse IgGl/CD3 | Mature T lymphocytes |
| Anti-HLA- | L243/mouse IgG2a/HLA- | Activated T cells, |
| DR | DR/MHC class II | macrophages, Langerhans |
| | | cells, cytokine-activated target |
| | | cells |
| Anti-TRR | L01.1/mouse | Cycling/DNA-synthetic cells |
| | IgG2a/transferrin receptors | |

(E) Assessment of Transferrin Receptor (TRR) Expression by Keratinocytes:

The expression of TRR on basal and suprabasal keratinocytes was quantified as the percentage of TRR+ keratinocytes per rete ridge. The keratinocytes of 10 consecutive rete ridges were assessed in for each biopsy and the results averaged.

(F) Assessment of Parakeratosis:

Parakeratosis, assessed histologically by the loss of granular layer and the presence of nuclei of immature keratinocytes within the stratum comeum, reflects in part the migratory capacity of basal keratinocytes towards the stratum comeum. The severity of parakeratosis is assessed as % involvement of a 4 mm linear strip of stratum comeum. An average of three sections per biopsy specimen was used for data analysis.

(G) Assessment of Epidermal T-cell Density:

The T-cell (CD8+ subset) population within the epidermal compartment represents the activated cytokine-secreting CD8+ T-cell population which has migrated from the vascular compartment in the dermis, across the basement membrane, into the epidermis. The density of the epidermal CD8+ T-cells was enumerated and quantified as the number of epidermal CD8+ T cells per hpf. This figure was the average of 10 hpf measurements per specimen.

(H) Assessment of HLA-DR+ Cells:

T-cell activation results in the expression of HLA-DR/MHC class II molecules by inflammatory cells (T-cells, macrophages, Langerhans cells). HLA-DR is also expressed by cytokine-activated target cells (C. E. Griffiths et al. (1991), supra). The activated HLA+ inflammatory cell population was assessed as abundant (200-500 or more cells/hpf), moderate (50-200 cells/hpf), and sparse (1-50/hpf). This figure was the average of 10 hpf measurements per specimen. Cytokine-activated non-inflammatory cells which express HLA-DR molecules in untreated psoriasis include epidermal keratinocytes and capillary endothelium. The expression of HLA-DR molecules on endothelial cells and/or keratinocytes was quantified as: (a) strongly positive (clumps of endothelial cells and/or keratinocytes showing HLA-DR positivity), (b) weakly positive (occasional endothelial cells and/or keratinocytes showing HLA-DR positivity) and (c) negative (no HLA-DR+ observed) based on assessments of 10 hpf/specimen.

(I) Statistical Analysis:

Comparison of the differences between mean measurements of the three patient groups were performed by analysis of variance (ANOVA) and Tukey's post-hoc test. The results were considered statistically significant when p<0.05 for the two-tailed test. All results in this report are expressed as mean±standard deviation (SD).

Results

Elevated phosphorylase kinase activity has been previously reported to correlate with increased psoriatic activity (M. C. Y. Heng et al. (1994), supra). To further study the role of phosphorylase kinase in active psoriasis with respect to PhK-dependent activities, i.e. cell cycling and cell migration, the experiments reported in this Example used (a) transferrin receptor (TRR)+ keratinocytes as a measure of keratinocyte cycling cell population); (b) parakeratosis as a marker of “surface” migration of immature keratinocyte population; (c) epidermal CD8+ T-cell population as a measure of migrated T-cell population. HLA-DR+/MHC class II+ on inflammatory and non-inflammatory cells was also assessed as a marker of cytokine-activity in psoriatic skin.

(A) Phosphorylase Kinase Activity in Active and Treated Psoriasis

The results are summarized in FIG. 10. Phosphorylase kinase activity was highest (1204.6±804.3 units/mg protein, mean±SD), in active/untreated psoriasis, with significantly lower levels in curcumin-treated resolving psoriasis (207.2±97.6 units/mg protein, p<0.0001), in Dovonex-treated resolved psoriasis (550.7±192.9 units/mg protein, p<0.01), and normal skin (105.4±44.6 units/mg protein, p<0.0001). Although the differnce did not reach statistical significance (p=0.11 for the two tailed test), phosphorylase kinase levels tended lower in curcumin-treated psoriasis than in Dovonex-treated psoriasis.

(B) Transferrin Receptor Positive (TRR+) Keratinocytes in Active and Treated Psoriasis

Transferrin and iron are required for the function of ribonucleotide reductase in the S phase of DNA sythesis. That TRR expression serves as a marker for DNA sythetic cells, i.e. cycling cells, is supported by studies showing that iron is required by ribonucleotide reductase for the S phase of DNA synthesis (C. E. Griffiths et al. (1991), supra; S. Eriksson et al., “Cell-Cycle Dependent Regulation of Mammalian Ribonucleotide Reductases. The S Phase Correlated Increase in Subunit M2 Is Regulated by De Novo Protein Synthesis,” J. Biol. Chem. 259: 11695-11700 (1984)). In activated T-cells, TRR expression is a prerequisite for stimulation of DNA synthesis by the T-cell growth factor, IL-2 (J. Laskey et al., “Evidence That Transferrin Supports Cell Proliferation by Supplying Iron for DNA Synthesis,” Exp. Cell Res. 176: 87-95 (1988)).

TRR+ keratinocytes were evaluated as a marker of cycling cells in active untreated psoriasis (n=10), and in resolving psoriatic lesions after treatment by the selective phosphorylase kinase inhibitor, curcumin (n=10), in resolving psoriatic lesions after treatment by the putative Type IIcAMP-dependent protein kinase stimulator, Dovonex (n=10), and in normal non-psoriatic skin (n=10). The percentage of TRR+ keratinocytes/rete ridge was highest (Mean=60.1%, DS=96.3; FIG. 11) in active/untreated psoriasis, with significantly lower values in curcumin-treated psoriasis (Mean-4.3%, SD=2.2), p<0.0001; FIG. 11), Dovonex-treated psoriasis (Mean=17.0%, SD=6.1), p<0.0001; FIG. 11), and normal skin (Mean=4.3%, SD=1.2), p<0.0001; FIG. 11). The TRR expression was significantly lower in curcumin-treated psoriasis than in Dovonex-treated psoriasis (p<0.001). The results are summarized in FIG. 11.

(C) Severity of Parakeratosis in Active and Treated Psoriasis

Marked parakeratosis (FIG. 14 (Panel A) was observed in all active psoriatic biopsises (n=10), with 94.6±4.3% (mean±SD) linear involvement of stratum corneum in untreated/active psoriasis, and absent to minimal involvement in curcumin-treated (1.4±2.4% (SD); p<0.0001; FIG. 14 (Panel B)), Dovonex-treated (8.4±6.8% (SD)); p<0.0001; FIG. 14 (Panel C)) psoriatic specimens, and absent in normal skin (FIG. 14 (Panel D)). The improvement in parakeratosis was significantly greater for the curcumin group than for the Dovonex group (p<0.01). The results are summarized in FIG. 12.

(D) Compartmentalized (Epidermal) T-cell Population in Active and Treated Psoriasis:

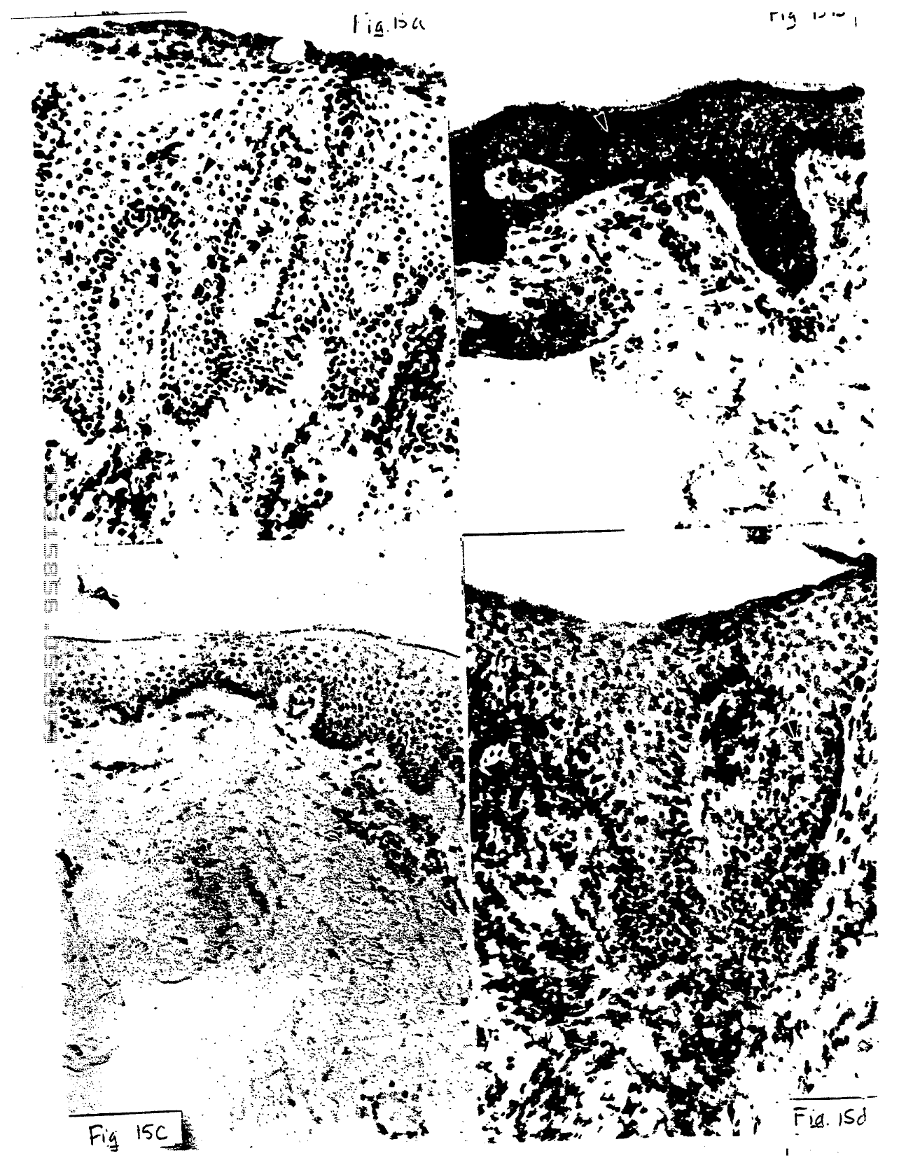

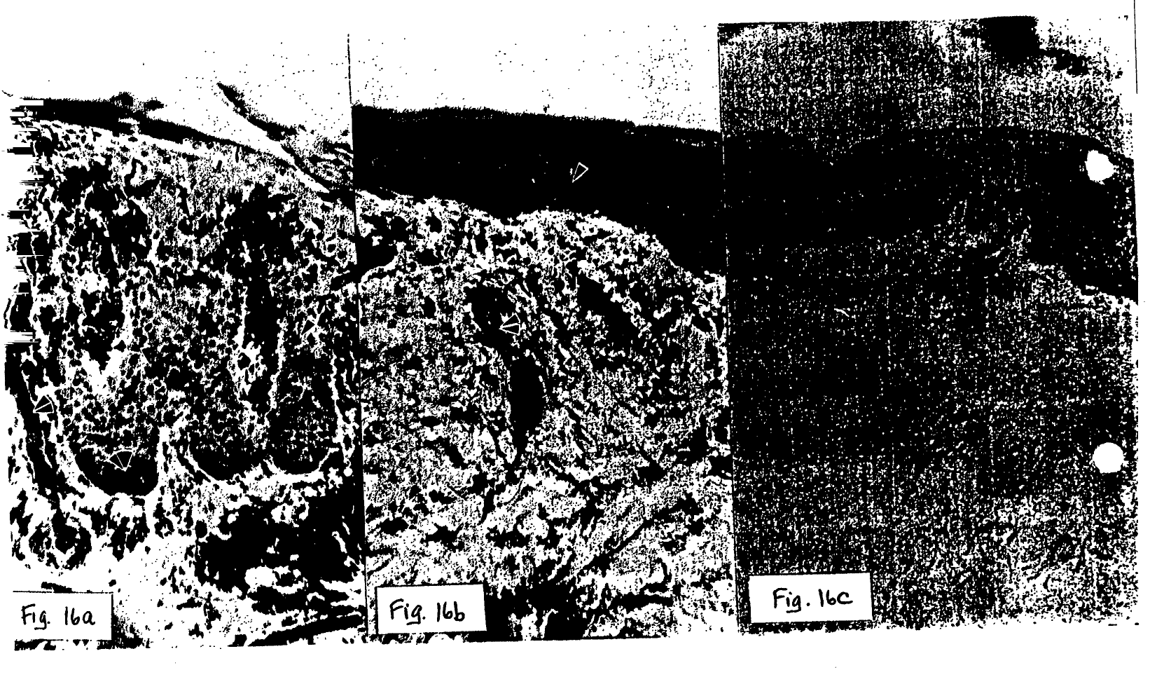

In evaluating cell-locomotion (phosphorylase kinase-dependent activity) in psoriatic biopsies, the compartmentalized (epidermal) CD8+ T-cell population was used as an indicator of T cells that migrated from the dermal vasculature into the epidermis. Since it has been reported that activated compartmentalized (epidermal) T cells in psoriatic skin release lymphokines that induce the psoriatic keratinocyte phenotype (O. Baadsgaard et al., “UM4D4+ (Cdw60) T Cells Are Compartmentalized in Psoriatic Skin and Release Lymphokines That Induce a Keratinocyte Phenotype Expressed in Psoriatic Skin Lesions,” J. Invest. Dermatol. 95: 275-282 (1990)), the epidermal CD8+ T-cell population can be used to assess the cytokine-secreting T cell population important in inducing the psoriatic phenotype. The density of the migratory T-cell population (number of epidermal CD8+ cells/hpf) was highest in active/untreated psoriasis (Mean=38.2, SD=6.1; FIG. 15 (Panel A)), with significantly decreased numbers in curcumin-treated resolving psoriasis (Mean=0.6, SD=0.8, p<0.0001; FIG. 15 (Panel B), and in Donovex-treated resolved psoriasis (Mean=8.7, SD=3.7, p<0.0001; FIG. 15 (Panel C). There were significantly fewer CD8+ lymphocytes in the curcumin group than in the Donovex group (p<0.0001). CD8+ cells were not observed in normal epidermis. Labeling with CD3 epitope shows that most of the CD3+ cells within the epidermis belong to the CD8+ subset (FIG. 15 (Panel D). The results are summarized in FIG. 13.

(E) HLA-DR Expression on Activated Inflammatory and Non-inflammatory Cells in Active and Treated Psoriasis

The expression of HLA-DR molecules is a reflection of the presence of cytokines, in particular interferon-K (C. E. Griffiths (1991), supra)), a cytokine secreted by activated T cells. In active untreated psoriatic skin, HLA-DR expression was strongly positive on both endothelial cells (FIG. 16 (Panel A)) and keratinocytes (FIG. 16 (Panel A)). HLA-DR expression on endothelial cells and keratinocytes was weakly positive in Dovonex-treated psoriasis (FIG. 16 (Panel B)), and not observed in curcumin-treated (FIG. 16 (Panel C) and normal skin. Overall, HLA-DR expression was as follows: (a) abundant in 10/10 skin specimens (FIG. 16 (Panel A)) from untreated psoriasis; (b) moderately abundant (FIG. 16 (Panel B) in 8/10 specimens, abundant in 1/10 specimens and sparse in 1/10 specimens from Dovonex-treated psoriasis; and (c) sparse (FIG. 16 (Panel C)) in 10/10 specimens from curcumin-treated psoriasis. HLA-DR+ cells were not observed in normal non-psoriatic skin.

Discussion

Psoriasis is an inherited disease, the casual mechanisms of which are still unclear. Recent studies suggest that at least two genes are implicated in the manifestations of psoriasis in predisposed individuals (H. Sigmundsdottir et al., “Circulating T Cells of Patients with Active Psoriasis Respond to Streptococcal M-Peptides Sharing Sequences with Human Epidermal Keratins,” Scand. J. Immunol. 45: 688-697 (1997); J. T. Elder et al., “The Genetics of Psoriasis,” Arch. Dermatol. 130: 216-224 (1994)). One of these is mapped to the short arm of the 6th chromosome encoding HLA genes (J. T. Elder et al. (1994), supra) and the other to the distal end of chromosme 17q (J. Tomfohrde et al., “Gene for Familial Psoriasis Susceptibility Mapped to the Distal End of Human Chromosome 17q,” Science 264: 1141-1145 (1994); R. P. Nair et al., “Evidence for Two Psoriasis Susceptibility Loci (HLA and 17q) and Two Novel Candidate Regions (16Q and 20p) by Genome-Wide Scan,” Hum. Mol. Genet. 6: 1349-1356 (1997)). Although details of the basic genetic defect remains to be identified, increased phosphorylase kinase activity in psoriatic epidermis has been previously reported to correlate with increased psoriatic activity (M. C. Y. Heng et al. (1994), supra. In addition, cAMP-dependence (C. L. Marcelo & J. J. Voorhees, “Cyclic Nucleotides and the Control of Psoriatic Cell Function,” Adv. Cyclic Nucleotide Res. 12: 1229-1237 (1980); J. Mendelsohn et al., “Inhibition of Human Lymphocyte Proliferation by Monoclonal Antibody to Transferrin Receptor,” Blood 62: 821-826 (1983)) and decreased type II cAMP levels (C. L. Marcelo & J. J. Voorhees (1980), supra; J. Mendelsohn et al. (1983), supra) have been reported to occur in psoriasis (S. Tournier et al., “Post-Translational Abnormality of the Type II Cyclic AMP-Dependent Protein Kinase in Psoriasis: Modulation by Retinoic Acid,” J. Cell. Biochem. 57: 647-654 (1995); S. Tournier et al., “Retinoylation of the Type II cAMP-Binding Regulatory Subunit of cAMP-Dependent Protein Kinase Is Increased in Psoriatic Human Fibroblasts,” J. Cell. Physiol. 167: 196-203 (1996)). Since type II cAMP-dependent protein kinase functions in deactivating phosphorylase kinase, the finding of low levels of Type II cyclic AMP-dependent protein kinase in psoriasis (M. C. Y. Heng et al. (1995), supra; S. Tournier et al. (1996), supra) provides support for the hypothesis that psoriatic activity may result from overactivity of phosphorylase kinase due to a defective deactivation or “switch off” mechanism.

In this study, the anti-psoriatic activity of curcumin is reported. Also known as diferuloylmethane, curcumin is a component in spices such as turmeric and ginger. This molecule has been reported to specifically inhibit phosphorylase kinase (S. Reddy & B. B. Aggarwal, “Curcumin Is a Non-Competitive and Selected Inhibitor of Phosphorylase Kinase,” FEBS Lett. 341: 19-22 (1994)). Comparing the anti-psoriatic effect of curcumin versus Dovonex, a drug with accepted clinical anti-psoriatic efficacy, the results reported in this Example show that curcumin is at least as effective as Dovonex in decreasing phosphorylase kinase activity, and in suppression of phosphorylase kinase-based functions, such as cell migration and cell proliferation. Based on clinical observations that untreated psoriatic plaques resolve within 4-6 weeks with curcumin and between 6-18 months or more with Dovonex, the results of this Example lead to the conclusion that curcumin is at least as effective as Dovonex in producing clinical resolution of psoriatic plaques.

The data in this Example also support the premise that the antipsoriatic effect of curcumin, as well as Dovonex, may be achieved through phosphorylase kinase inhibition. The molecular structure of phosphorylase kinase, described above, is relevant to a better understanding of the action of these drugs. The enzyme is activated by binding of Ca++ to the A subunit (calmodulin) through rises in intracellular Ca²⁺ (C. O. Brostrom et al., “The Relation of Skeletal Muscle Phosphorylase Kinase Activity to Ca²⁺,” J. Biol. Chem. 246:1961-1967 (1971)), or through a phosphorylation reaction catalyzed by protein kinase C (D. A. Walsh et al., “Catalysis of the Phosphorylase Kinase Activation Reaction,” J. Biol. Chem. 246: 1968-1976 (1971)). The enzyme is under both hormonal and neuronal control (P. Cohen, “The Role of Protein Phosphorylation in Neural and Hormonal Control of Cellular Activity,” Nature 296: 613-620; J. J. Davidson et al. (1992), supra). The α and β subunits contain phosphorylation sites for protein kinase A, i.e. these sites are phosphorylated by separate cAMP-dependent enzymes. These cAMP-dependent enzymes are crucial for the activity of phosphorylase kinase, since phosphorylase kinase is also activated by phosphorylation of the β subunit, a reaction catalyzed by Type I cAMP-dependent protein kinase, and deactivated by phosphorylation of its I subunit, a reaction catalyzed by Type II cyclic AMP-dependent protein kinase.

The δ subunit is the functional catalytic unit (M. Dasgupta & D. K. Blumenthal, “Characterization of the Regulatory Domain of the γ Subunit of Phosphorylase Kinase: the Two Noncontiguous Calmodulin-Binding Subdomains Are Also Autoinhibitory,” J. Biol. Chem. 270: 22283-22289 (1995)). Since curcumin has been shown to be a selective phosphorylase kinase inhibitor (S. Reddy & B. B. Aggarwal (1994), supra), it is possible that the curcumin molecule could serve as a pseudosubstrate by directly binding to the regulatory subdomains of the catalytic δ subunit (M. Dasgupta & D. K. Blumenthal (1995), supra). The molecular structure of curcumin suggests that it may overlap the phosphorylation site on the β subunit, i.e. the site of action of Type I cAMP-dependent protein kinase (C. O. Brostrom et al. (1971), supra). This conclusion is supported by observations that the inhibitory effect of curcumin on phosphorylase kinase is achieved, at least in part, through the action of curcumin on Type I cAMP-dependent protein kinase (M. Hasmeda & G. M. Polya, “Inhibition of Cyclic AMP-Dependent Protein Kinase by Curcumin,” Phytochemistry 42: 599-605 (1996)).

The data in this Example support observations by others that phosphorylase kinase may have wider substrate specificity than previously appreciated (C. J. Yuan et al., “Phosphorylase Kinase, a Metal Ion-Dependent Dual Specificity Kinase,” J. Biol. Chem. 268: 17683-17686 (1993); T. S. Huang et al., “The Amino Acid Sequences of the Phosphorylated Sites in Troponin-I from Rabbit Skeletal Muscle,” FEBS Lett. 42: 249-252 (1974); T. G. Sotiroudis & T. P. Geladopoulos (1992), supra). Thus, phosphorylase kinase phosphorylates key molecules in various pathways, leading to various functional effects on psoriatic activity. Phosphorylation of serine residues on glycogen phosphorylase and phosphorylase b generates ATP through glycogenolysis (P. Cohen, “The Role of cAMP-Dependent Protein Kinase in the Regulation of Glycogen Metabolism in Mammalian Skeletal Muscle,” Curr. Top. Cell. Regul. 14: 117-196 (1978); P. Cohen (1982), supra; B. Harmann et al., “Isoform Diversity of Phosphorylase Kinase α and β Subunits Generated by Alternative RNA Splicing,” J. Biol. Chem. 266: 15631 (1991)), thus supplying ATP for various cellular activities. In addition, by phosphorylating inositol in phosphatidylinositol (Z. Georgeoussi & L. M., Jr. (1986), supra), and by binding of Ca²⁺ to its calmodulin A subunit (C. O. Brostrom et al. (1971), supra), phosphorylase kinase links calcium-calmodulin-dependent and inositol-dependent signaling pathways, such as those triggered by extrinsic stimuli (trauma, allergens, and infectious organisms) to signaling pathways involved in gene transcription. Phosphorylase linase is also involved in phosphorylating myosin to expose actin binding sites to form acto-myosin contractile fibers in migration of non-muscle cells (M. F. Carlier (1991), supra), such as inflammatory cells and epidermal keratinocytes. This premise is supported by previous observations of rapid migration of CD8+ lymphocytes into the epidermis as early as 2-5 mins following tape-stripping (M. C. Y. Heng et al. (1995), supra; M. C. Y. Heng et al. (1991), supra). By also phosphorylating tyrosine kinase (C. J. Yuan et al. (1993), supra), a crucial enzyme in tyrosine kinase-based growth factor receptors, phosphorylase kinase links extrinsic signaling pathways to pathways modulated by cytokines and growth factors. In psoriasis, phosphorylase kinase thus integrates glycogenolysis and ATP production (M. C. Y. Heng et al. (1994), supra) to energy-dependent processes such as (a) T-cell activation, (c) inflammatory and non-inflammatory cell migration, and (c) growth factor-dependent proliferation of T cells and keratinocytes. The data in this Example showing the correlation of phosphorylase kinase activity to keratinocyte cycling (TRR+) population, compartmentalized (epidermal) T-cell (CD8+) population in active and treated/resolving psoriasis supports the above concepts.

The current evidence support the premise that defective deactivation of phosphorylase kinase, perhaps due to low levels of Type II cAMP-dependent protein kinase, may be the fundamental abnormality in psoriasis (S. Tournier et al. (1995), supra; S. Tournier et al. (1996), supra). Low levels of Type II cyclic AMP protein kinase levels, attributed to a post-translational abnormality of the enzyme that is partially reversible by drug therapy, have been observed in psoriasis (S. Tournier et al. (1995), supra; S. Tournier et al. (1996), supra). By showing that the anti-psoriatic effect of Dovonex (vitamin D3 analogue), a putative type II cAMP-dependent protein kinase stimulator (M. Sikorsia & J. F. Whitfield, “The Regulatory and Catalytic Subunits of Rat Liver Cyclic AMP-Dependent Protein Kinases Respond Differently to Thyroparathyroidectomy and 1α,25-Dihydroxyvitamin D₃,” Biochem. Biophys. Res. Commun. 129: 766-772 (1985)), may be achieved also through inhibition of phosphorylase kinase activity, the data in this Example provides support for the phosphorylase kinase inhibitory role of Type II cAMP-dependent protein kinase. The observed protection of calcium channel blockers against the development of psoriasis induced by the Koebner phenomenon (M. C. Y. Heng & S. G. Allen (1992), supra) support a calmodulin-containing molecule involved in psoriatic activity, as does the antipsoriatic properties of calmodulin inhibitors, such as cyclosporin A and anthralin (W. F. G. Tucker et al., “Biological Active Calmodulin Levels Are Elevated in Both Involved and Uninvolved Epidermis in Psoriasis,” J. Invest. Dermatol. 82: 298-299 (1984); S. MacNeil et al., “Antiproliferative Effects on Keratinocytes of a Range of Clinically Used Drugs with Calmodulin Antagonist Activity,” Br. J. Dermatol. (1993)). Evidence also suggest that retinoids act by stimulating cAMP-dependent protein kinase (O. Holian & R. Kumar, “Cyclic AMP and Cyclic AMP-Dependent Protein Kinase in Mouse Skin. II. In Vitro Effects of Isotrenitoin and Etretinate,” Arch. Derm. Res. 287: 161-164 (1985)). The putative sites of action of these antipsoriatic drugs relative to phosphorylase kinase activity is summarized in FIG. 1.

It is interesting to speculate whether the elevated phosphorylase kinase activity is the result of intrinsic/genetic abnormalities of the phosphorylase kinase subunits in psoriatic patients, or secondary to intrinsic abnormalities of the cAMP-dependent protein kinase regulatory enzymes. Genetic studies in psoriatic individuals show linkage of susceptibility loci encoding HLA genes mapped to chromosome 6, and chromosome 17 (17q) (J. Tomfohrde et al. (1994), supra; R. P. Nair et al. (1997), supra), with candidate loci also found on 16q (R. P. Nair et al. (1997), supra). Of interest is the observation that the regulatory subunit (RIA) of Type II cAMP-dependent protein kinase is encoded by genes residing on chromosome 17 (G. Sozzi et al., “A t(10:17) Translocation Creates the RET/PTC2 Chimeric Transforming Sequence in Papillary Thyroid Carcinoma,” Genes Chromosomes & Cancer 9: 244-250 (1994)). The incrimination of susceptibility loci on chromosome 16 in psoriasis may also be relevant since genes encoding phosphorylase kinase l subunits have been mapped also to the distal end of chromosome 16 (U. Francke et al., “Assignment of Human Genes for Phosphorylase Kinase Subunits α (PHKA) to Xq12-q13 and β (PHKB) to 16q12-13,” Am. J. Hum. Genet. 45: 276-282 (1989)).

The results of this Example indicate the role of phosphorylase kinase in increased migratory activity of inflammatory cells into uninvolved psoriatic skin and also in the increased migratory activity of keratinocytes. Thus, this Example supports the use of curcumin, curcumin derivatives, or curcuminoids to control or block the increased migratory activity of these cells, both in psoriasis and in other conditions in which such increased migratory activity occurs.

### ADVANTAGES OF THE PRESENT INVENTION

The present invention provides a more efficient way to administer curcumin, curcuminoids, and curcumin derivatives in active form to treat a number of conditions and diseases. The conditions and diseases treatable by the method of the present invention include:

(1) dermatological and mucosal inflammatory diseases, such as psoriasis, periodontal disease, gingivitis, sinusitis, hay fever, periodontitis, neuritis, skin wounds, bums and scalds, chemical-, radiation-, and sun-induced injury to the skin, inflammation of the ear, nose, or throat, vaginitis, proctitis, allergic and hypersensitive reactions, smoking-induced premature skin aging, eczemas, and skin infections (bacterial, viral, fungal, or mycoplasmal);

(2) inflammatory diseases such as arthritis, systemic lupus erythematosus (SLE), connective tissue diseases, atherosclerosis, the inflammatory process that occurs during partial or complete blockage of an artery such as a coronary artery, Alzheimer's Disease, gastritis, chronic hepatitis, chronic diverticulitis, osteomyelitis, inflammatory bowel diseases such as colitis and Crohn's disease, pelvic inflammatory disease, chronic prostatitis, sinusitis, and radiation- and smoking-induced injury, including premature atherosclerosis;

(3) benign and malignant tumors, including metastatic tumors (breast, prostate, lung, skin, melanomas, brain, liver, pancreas, gastric, intestinal, colonic, kidney, bladder, cervix, ovary, uterus, central nervous system, sinuses, eye, ear, bone, or thyroid) or lymphomas and leukemias; and

(4) infections, such as infections caused by bacteria, superficial and deep fungi (dermatophytes, sporotrichium, histoplasma, blastomyces), mycoplasmas, viruses (including herpes simplex virus, varicella zoster virus, adenovirus, and human immunodeficiency virus), and parasites (nematodes, other worms, and other pathogenic parasites, such as organisms causing filariasis, schistosomiasis, and malaria).

The methods and compositions according to the present invention are usable with other therapeutic agents and methods and are well tolerated by patients.

Although the present invention has been described with considerable detail, with reference to certain preferred versions thereof, other versions and embodiments are possible. Therefore, the scope of the invention is determined by the following claims.

I claim:

1. A method for treating inflammation in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal.

2. The method of claim 1 wherein the at least one alcohol is selected from the group consisting of alcohols with from 1 to 6 carbon atoms.

3. The method of claim 2 wherein the at least one alcohol is selected from the group consisting of alcohols with from 1 to 3 carbon atoms.

4. The method of claim 1 wherein the at least one alcohol is saturated.

5. The method of claim 1 wherein the at least one alcohol is monohydric.

6. The method of claim 5 wherein the at least one alcohol is selected from the group consisting of ethanol, 1-propanol, and 2-propanol.

7. The method of claim 6 wherein the at least one alcohol is ethanol.

8. The method of claim 1 wherein the mammal is a human being.

9. The method of claim 1 wherein the mammal is a socially or economically important animal selected from the group consisting of a cow, a horse, a sheep, a pig, a goat, a dog, and a cat.

10. The method of claim 1 wherein at least one of the following stages of inflammation is inhibited by the administration of soluble curcumin: (1) the migration of γ/δ T cells occurring at about 30 minutes to about 4 hours after the inflammatory stress; (2) the migration of neutrophils beginning at about 18-24 hours after the inflammatory stress;(3) the migration of macrophages beginning at about 24 hours after the inflammatory stress; and (4) the migration of α/β T cells and other cells such as eosinophils beginning at about 48 hours to 72 hours after the inflammatory stress.

11. The method of claim 1 wherein the curcumin is administered as a boron complex.

12. The method of claim 11 wherein the boron complex is selected from the group consisting of
(a) a difluoroboron complex;
(b) a mixed complex in which the two fluorine atoms of a difluoroboron complex are replaced with the carboxyl oxygens of oxalic acid;
(c) a mixed complex in which the two fluorine atoms of a difluoroboron complex are replaced with a carboxyl group and a hydroxyl group of citric acid;
(d) a mixed complex in which the two fluorine atoms of a difluoroboron complex are replaced with the two hydroxyl groups of dibenzyl tartramide; and
(e) a mixed complex in which the two fluorine atoms of a difluoroboron complex are replaced with a second molecule of curcumin.

13. The method of claim 1 wherein the curcumin is administered in a liposome.

14. The method of claim 13 wherein the curcumin administered in a liposome is administered in a preparation selected from the group consisting of a skin preparation, an eye drop preparation, a nasal drop preparation, an oral preparation, a pharyngeal preparation, a rectal preparation, a vaginal preparation, a bladder preparation, a urethral preparation, a bronchial preparation, and a parenteral preparation.

15. A method for treating inflammation in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin, a soluble curcuminoid, or a soluble curcumin derivative in a solution containing at least one alcohol to a mammal to detectably inhibit the activity of phosphorylase kinase in the blood of the mammal or in a tissue of the mammal.

16. The method of claim 15 wherein the curcumin, curcuminoid, or curcumin derivative is selected from the group consisting of:
(a) curcumin;
(b) a curcuminoid of formula (I) in which:
(i) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;
(ii) R₁ is —H; R₂ is —OH; R₃ is —H or —OH; R₄ is —H, R₅ is —H; R₆ is —OH; and R₇ is —H or —OH;
(iii) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;
(iv) R₁ is —OH, R₂ is —OH, R₃ is —OH, R₄ is —H or —OH, R₅ is —OH, R₆ is —OH; and R₇ is —OH;
(v) R₁ is —OCH₃; R₂ is —OCH₃; R₃ is —OCH₃; R₄ is —H or —OH; R₅ is —OCH₃; R₆ is —OCH₃; and R₇ is —OCH₃;
(vi) R₁ is —H; R₂ is —OCH₃; R₃ is —OCH₃; R₄ is —H or —OH; R₅ is —H; R₆ is —OCH₃; and R₇ is —OCH₃;
(vii) R₁ is —H; R₂ is —OH; R₃ is —H; R₄ is —H; R₅ is —H or —OH; R₆ is —OH; and R₇ is —H;
(viii) R₁ is —H; R₂ is —OCH₃; R₃ is —H; R₄ is —H; R₅ is —H or —OH; R₆ is —OCH₃; and R₇ is —H; or
(ix) R₁ is —OH; R₂ is —OCH₃; R₃ is —H or —OH; R₄ is H or —OH; R₅ is —OH; R₆ is —OCH₃; and R₇ is —H or —OH; 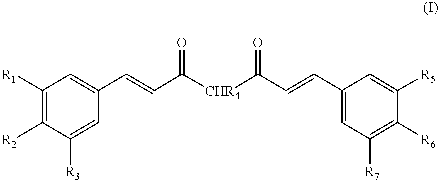
(c) a curcuminoid of formula (II) in which the alternatives for R₁ through R₇ are the same as those recited in paragraph (b); 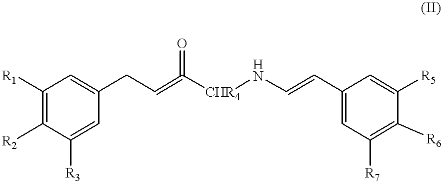
(d) a curcuminoid of formula (III) in which the alternatives for R₁ through R₇ are the same as those recited in paragraph (b); 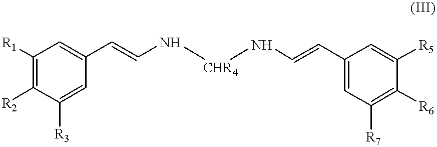
(e) the compound of formula (IV) in which X is —H, the compound being designated furfural curcuminoid;
(f) an analogue of furfural curcuminoid in which X is —OH, ethyl, methyl, or acetyl; 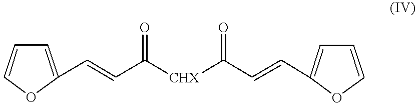
(g) the compound of formula (V) in which X is —H, the compound being designated salicyl curcuminoid;
(h) an analogue of salicyl curcuminoid in which X is —OH, ethyl, methyl, or acetyl; 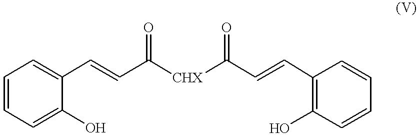
(i) the compound of formula (VI) in which X is —H, the compound being designated veratryl curcuminoid;
(j) an analogue of veratryl curcuminoid in which X is —OH, ethyl, methyl, or acetyl; 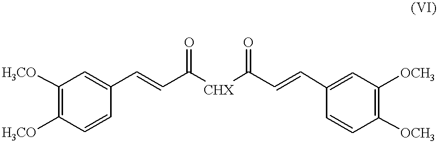
(k) the compound of formula (VII) in which X is —H, the compound being designated p-anisyl curcuminoid;
(l) an analogue of p-anisyl curcuminoid in which X is —OH, ethyl, methyl, or acetyl; 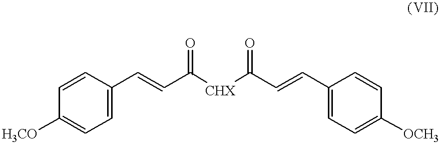
(m) the compound of formula (VIII) in which X is —H, the compound being designated piperonal curcuminoid;
(n) an analogue of piperonal curcuminoid in which X is —OH, ethyl, methyl, or acetyl; 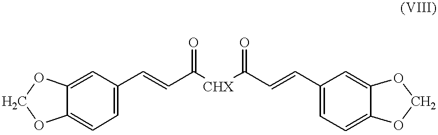
(o) a tetrahydrocurcuminoid of formula (IX) in which the alternatives for R₁ through R₇ are the same as those recited in paragraph (b); 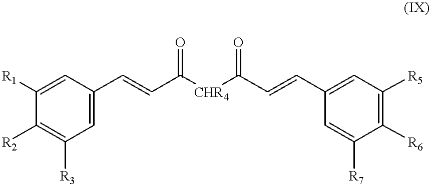
(p) a curcuminoid of formula (X) in which the alternatives for R₁ through R₇ are the same as those recited in paragraph (b); 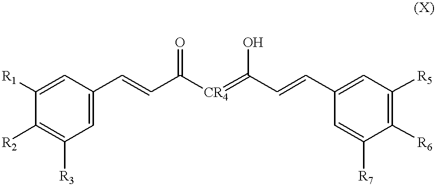
(q) a curcuminoid of formula (XI) in which the alternatives for R₁ through R₇ are the same as those recited in paragraph (b); 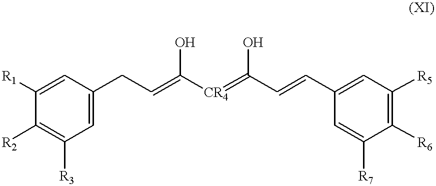
(r) a reduced curcuminoid of formula (XII) in which the alternatives for R₁ through R₇ are the same as those recited in paragraph (b); 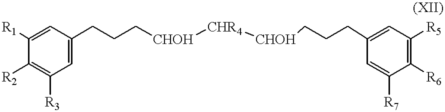
(s) derivatives of the compounds recited in (b) through (r) in which any of the methoxy groups are replaced with lower alkoxy groups selected from the group consisting of ethoxy, n-propoxy, and isopropoxy;
(t) derivatives of the compounds recited in (b) through (r) in which any of the hydroxy groups of the phenolic moieties are substituted with an acyl group selected from the group consisting of acetyl, propionyl, butyryl, and isobutyryl;
(u) analogues of the compounds recited in (b), (c), and (e) through (p) in which one or both of the carbonyl (CO) groups are replaced by amino (NH) groups in analogy with formulas II and III; and
(v) analogues of the compounds recited in (b), (c), and (e) through (p) in which one or both of the oxygens of the carbonyl groups are replaced by sulfur to form thiocarbonyl groups.

17. The method of claim 15 wherein the at least one alcohol is selected from the group consisting of alcohols with from 1 to 6 carbon atoms.

18. The method of claim 17 wherein the at least one alcohol is selected from the group consisting of alcohols with from 1 to 3 carbon atoms.

19. The method of claim 15 wherein the at least one alcohol is saturated.

20. The method of claim 15 wherein the at least one alcohol is monohydric.

21. The method of claim 18 wherein the at least one alcohol is selected from the group consisting of ethanol, 1-propanol, and 2-propanol.

22. The method of claim 21 wherein the at least one alcohol is ethanol.

23. The method of claim 15 wherein the mammal is a human.

24. The method of claim 15 wherein the mammal is a socially or economically important animal selected from the group consisting of a cow, a horse, a sheep, a pig, a goat, a dog, and a cat.

25. The method of claim 15 wherein at least one of the following stages of inflammation is inhibited by the administration of soluble curcumin, a soluble curcumin derivative, or a soluble curcuminoid: (1) the migration of γ/δ T cells occurring at about 30 minutes to about 4 hours after the inflammatory stress; (2) the migration of neutrophils beginning at about 18-24 hours after the inflammatory stress; (3) the migration of macrophages beginning at about 24 hours after the inflammatory stress; and (4) the migration of α/β T cells and other cells such as eosinophils beginning at about 48 hours to 72 hours after the inflammatory stress.

26. The method of claim 15 wherein the soluble curcumin, a soluble curcuminoid, or a soluble curcumin derivative is administered as a boron complex.

27. The method of claim 26 wherein the boron complex is selected from the group consisting of
(a) a difluoroboron complex;
(b) a mixed complex in which the two fluorine atoms of a difluoroboron complex are replaced with the carboxyl oxygens of oxalic acid;
(c) a mixed complex in which the two fluorine atoms of a difluoroboron complex are replaced with a carboxyl group and a hydroxyl group of citric acid;
(d) a mixed complex in which the two fluorine atoms of a difluoroboron complex are replaced with the two hydroxyl groups of dibenzyl tartramide; and
(e) a mixed complex in which the two fluorine atoms of a difluoroboron complex are replaced with a second molecule of curcumin, a curcumin derivative, or a curcuminoid.

28. The method of claim 15 wherein the soluble curcumin, a soluble curcuminoid, or a soluble curcumin derivative is administered in a liposome.

29. The method of claim 28 wherein the curcumin, curcumin derivative, or curcuminoid administered in a liposome is administered in a preparation selected from the group consisting of a skin preparation, an eye drop preparation, a nasal drop preparation, an oral preparation, a pharyngeal preparation, a rectal preparation, a vaginal preparation, a bladder preparation, a urethral preparation, a bronchial preparation, and a parenteral preparation.

30. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal, the condition or disease being selected from the group consisting of: psoriasis, skin wounds, bums and scalds, scars, chemical-, radiation-, and sun-induced injury to the skin, smoking-induced injury to the skin, allergic and hypersensitive reactions, hay fever, periodontal disease, gingivitis, eczemas, and skin infections (bacterial, viral, fungal, or mycoplasmal).

31. The method of claim 30 wherein the mammal is a human.

32. The method of claim 30 wherein the mammal is a socially or economically important animal selected from the group consisting of a cow, a horse, a sheep, a goat, a pig, a dog, and a cat.

33. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal, the condition or disease being selected from the group consisting of: arthritis, systemic lupus erythematosus (SLE), connective tissue diseases, atherosclerosis, Alzheimer's Disease, the inflammatory process that occurs during partial or complete blockage of an artery such as a coronary artery, gastritis, chronic hepatitis, chronic diverticulitis, osteomyelitis, inflammatory bowel diseases, pelvic inflammatory disease, chronic prostatitis, sinusitis, neuritis, neuropathies, and radiation- and smoking-induced injury.

34. The method of claim 33 wherein the mammal is a human.

35. The method of claim 33 wherein the mammal is a socially or economically important animal selected from the group consisting of a cow, a horse, a sheep, a goat, a pig, a dog, and a cat.

36. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal, the condition or disease being selected from the group consisting of benign and malignant tumors, including metastatic tumors, of a tissue selected from the group consisting of breast, prostate, lung, skin, melanomas, brain, liver, pancreas, gastric, intestinal, colon, kidney, bladder, cervix, ovary, uterus, central nervous system, sinuses, eye, ear, bone, and thyroid, lymphomas and leukemias.

37. The method of claim 36 wherein the mammal is a human.

38. The method of claim 36 wherein the mammal is a socially or economically important animal selected from the group consisting of a cow, a horse, a sheep, a goat, a pig, a dog, and a cat.

39. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal, the condition or disease being selected from the group consisting of infections caused by bacteria, superficial fungi, deep fungi, viruses, mycoplasmas, and parasites.

40. The method of claim 39 wherein the mammal is a human.

41. The method of claim 39 wherein the mammal is a socially or economically important animal selected from the group consisting of a cow, a horse, a sheep, a goat, a pig, a dog, and a cat.

42. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal, the condition or disease being diabetes.

43. The method of claim 42 wherein the mammal is a human.

44. The method of claim 42 wherein the mammal is a socially or economically important animal selected from the group consisting of a cow, a horse, a sheep, a goat, a pig, a dog, and a cat.

45. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin in a solution containing at least one alcohol to a mammal to detectably inhibit the activity of phosphorylase kinase in the blood of the mammal or in a tissue of the mammal, the condition or disease being a neurodegenerative condition.

46. The method of claim 45 wherein the mammal is a human.

47. The method of claim 45 wherein the mammal is a socially or economically important animal selected from the group consisting of a cow, a horse, a sheep, a goat, a pig, a dog, and a cat.

48. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin, a soluble curcuminoid, or a soluble curcumin derivative in a solution containing at least one alcohol to a mammal to detectably inhibit the activity of phosphorylase kinase in the blood of the mammal or in a tissue of the mammal, the condition or disease being selected from the group consisting of: psoriasis, skin wounds, burns and scalds, scars, chemical-, radiation-, and sun-induced injury to the skin, smoking-induced injury to the skin, allergic and hypersensitive reactions, hay fever, periodontal disease, gingivitis, eczemas, and skin infections (bacterial, viral, fungal, or mycoplasmal).

49. The method of claim 48 wherein the curcumin, curcuminoid, or curcumin derivative is selected from the group consisting of:
(a) curcumin;
(b) a curcuminoid of formula (I) in which:
(i) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;
(ii) R₁ is —H; R₂ is —OH; R₃ is —H or —OH; R₄ is —H, R₅ is —H; R₆ is —OH; and R₇ is —H or —OH;
(iii) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;
(iv) R₁ is —OH, R₂ is —OH, R₃ is —OH, R₄ is —H or —OH, R₅ is —OH, R₆ is —OH; and R₇ is —OH;
(v) R₁ is —OCH₃; R₂ is —OCH₃; R₃ is —OCH₃; R₄ is —H or —OH; R₅ is —OCH₃; R₆ is —OCH₃; and R₇ is —OCH₃;
(vi) R₁ is —H; R₂ is —OCH₃; R₃ is —OCH₃; R₄ is —H or —OH; R₅ is —H; R₆ is —OCH₃; and R₇ is —OCH₃;
(vii) R₁ is —H; R₂ is —OH; R₃ is —H; R₄ is —H; R₅ is —H or —OH; R₆ is —OH; and R₇ is —H;
(viii) R₁ is —H; R₂ is —OCH₃; R₃ is —H; R₄ is —H; R₅ is —H or —OH; R₆ is —OCH₃; and R₇ is —H; or
(ix) R₁ is —OH; R₂ is —OCH₃; R₃ is —H or —OH; R₄ is H or —OH; R₅ is —OH; R₆ is —OCH₃; and R₇ is —H or —OH;
(c) a curcuminoid of formula (II) in which the alternatives for R₁ through R₇ are the same as those recited in paragraph (b);
(d) a curcuminoid of formula (III) in which the alternatives for R₁ through R₇ are the same as those recited in paragraph (b);
(e) the compound of formula (IV) in which X is —H, the compound being designated furfural curcuminoid;
(f) an analogue of furfural curcuminoid in which X is —OH, ethyl, methyl, or acetyl;
(g) the compound of formula (V) in which X is —H, the compound being designated salicyl curcuminoid;
(h) an analogue of salicyl curcuminoid in which X is —OH, ethyl, methyl, or acetyl;
(i) the compound of formula (VI) in which X is —H, the compound being designated veratryl curcuminoid;
(j) an analogue of veratryl curcuminoid in which X is —OH, ethyl, methyl, or acetyl;
(k) the compound of formula (VII) in which X is —H, the compound being designated p-anisyl curcuminoid;
(l) an analogue of p-anisyl curcuminoid in which X is —OH, ethyl, methyl, or acetyl;
(m) the compound of formula (VIII) in which X is —H, the compound being designated piperonal curcuminoid;
(n) an analogue of piperonal curcuminoid in which X is —OH, ethyl, methyl, or acetyl;
(o) a tetrahydrocurcuminoid of formula (IX) in which the alternatives for R₁ through R₇ are the same as those recited in paragraph (b);
(p) a curcuminoid of formula (X) in which the alternatives for R₁ through R₇ are the same as those recited in paragraph (b);
(q) a curcuminoid of formula (XI) in which the alternatives for R₁ through R₇ are the same as those recited in paragraph (b);
(r) a reduced curcuminoid of formula (XII) in which the alternatives for R₁ through R₇ are the same as those recited in paragraph (b);
(s) derivatives of the compounds recited in (b) through (r) in which any of the methoxy groups are replaced with lower alkoxy groups selected from the group consisting of ethoxy, n-propoxy, and isopropoxy;
(t) derivatives of the compounds recited in (b) through (r) in which any of the hydroxy groups of the phenolic moieties are substituted with an acyl group selected from the group consisting of acetyl, propionyl, butyryl, and isobutyryl;
(u) analogues of the compounds recited in (b), (c), and (e) through (p) in which one or both of the carbonyl (CO) groups are replaced by amino (NH) groups in analogy with formulas II and III; and
(v) analogues of the compounds recited in (b), (c), and (e) through (p) in which one or both of the oxygens of the carbonyl groups are replaced by sulfur to form thiocarbonyl groups.

50. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin, a soluble curcuminoid, or a soluble curcumin derivative in a solution containing at least one alcohol to a mammal to detectably inhibit the activity of phosphorylase kinase in the blood of the mammal or in a tissue of the mammal, the condition or disease being selected from the group consisting of: arthritis, systemic lupus erythematosus (SLE), connective tissue diseases, atherosclerosis, Alzheimer's Disease, the inflammatory process that occurs during partial or complete blockage of an artery such as a coronary artery, gastritis, chronic hepatitis, chronic diverticulitis, osteomyelitis, inflammatory bowel diseases, pelvic inflammatory disease, chronic prostatitis, sinusitis, neuritis, neuropathies, and radiation- and smoking-induced injury.

51. The method of claim 50 wherein the curcumin, curcuminoid, or curcumin derivative is selected from the group consisting of:
(a) curcumin;
(b) a curcuminoid of formula (I) in which:
(i) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;
(ii) R₁ is —H; R₂ is —OH; R₃ is —H or —OH; R₄ is —H, R₅ is —H; R₆ is —OH; and R₇ is —H or —OH;
(iii) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;
(iv) R₁ is —OH, R₂ is —OH, R₃ is —OH, R₄ is —H or —OH, R₅ is —OH, R₆ is —OH; and R₇ is —OH;
(v) R₁ is —OCH₃; R₂ is —OCH₃; R₃ is —OCH₃; R₄ is —H or —OH; R₅ is —OCH₃; R₆ is —OCH₃; and R₇ is —OCH₃;
(vi) R₁ is —H; R₂ is —OCH₃; R₃ is —OCH₃; R₄ is —H or —OH; R₅ is —H; R₆ is —OCH₃; and R₇ is —OCH₃;
(vii) R₁ is —H; R₂ is —OH; R₃ is —H; R₄ is —H; R₅ is —H or —OH; R₆ is —OH; and R₇ is —H;
(viii) R₁ is —H; R₂ is —OCH₃; R₃ is —H; R₄ is —H; R₅ is —H or —OH; R₆ is —OCH₃; and R₇ is —H; or
(ix) R₁ is —OH; R₂ is —OCH₃; R₃ is —H or —OH; R₄ is H or —OH; R₅ is —OH; R₆ is —OCH₃; and R₇ is —H or —OH;
(c) a curcuminoid of formula (II) in which the alternatives for R₁ through R₇ are the same as those recited in paragraph (b);
(d) a curcuminoid of formula (III) in which the alternatives for R₁ through R₇ are the same as those recited in paragraph (b);
(e) the compound of formula (IV) in which X is —H, the compound being designated furfural curcuminoid;
(f) an analogue of furfural curcuminoid in which X is —OH, ethyl, methyl, or acetyl;
(g) the compound of formula (V) in which X is —H, the compound being designated salicyl curcuminoid;
(h) an analogue of salicyl curcuminoid in which X is —OH, ethyl, methyl, or acetyl;
(i) the compound of formula (VI) in which X is —H, the compound being designated veratryl curcuminoid;
(j) an analogue of veratryl curcuminoid in which X is —OH, ethyl, methyl, or acetyl;
(k) the compound of formula (VII) in which X is —H, the compound being designated p-anisyl curcuminoid;
(l) an analogue of p-anisyl curcuminoid in which X is —OH, ethyl, methyl, or acetyl;
(m) the compound of formula (VIII) in which X is —H, the compound being designated piperonal curcuminoid;
(n) an analogue of piperonal curcuminoid in which X is —OH, ethyl, methyl, or acetyl;
(o) a tetrahydrocurcuminoid of formula (IX) in which the alternatives for R₁ through R₇ are the same as those recited in paragraph (b);
(p) a curcuminoid of formula (X) in which the alternatives for R₁ through R₇ are the same as those recited in paragraph (b);
(q) a curcuminoid of formula (XI) in which the alternatives for R₁ through R₇ are the same as those recited in paragraph (b);
(r) a reduced curcuminoid of formula (XII) in which the alternatives for R₁ through R₇ are the same as those recited in paragraph (b);
(s) derivatives of the compounds recited in (b) through (r) in which any of the methoxy groups are replaced with lower alkoxy groups selected from the group consisting of ethoxy, n-propoxy, and isopropoxy;
(t) derivatives of the compounds recited in (b) through (r) in which any of the hydroxy groups of the phenolic moieties are substituted with an acyl group selected from the group consisting of acetyl, propionyl, butyryl, and isobutyryl;
(u) analogues of the compounds recited in (b), (c), and (e) through (p) in which one or both of the carbonyl (CO) groups are replaced by amino (NH) groups in analogy with formulas II and III; and
(v) analogues of the compounds recited in (b), (c), and (e) through (p) in which one or both of the oxygens of the carbonyl groups are replaced by sulfur to form thiocarbonyl groups.

52. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin, a soluble curcuminoid, or a soluble curcumin derivative in a solution containing at least one alcohol to a mammal to detectably inhibit the activity of phosphorylase kinase in the blood of the mammal or in a tissue of the mammal, the condition or disease being selected from the group consisting of benign and malignant tumors, including metastatic tumors, of a tissue selected from the group consisting of breast, prostate, lung, skin, melanomas, brain, liver, pancreas, gastric, intestinal, colon, kidney, bladder, cervix, ovary, uterus, central nervous system, sinuses, eye, ear, bone, and thyroid, lymphomas and leukemias.

53. The method of claim 52 wherein the curcumin, curcuminoid, or curcumin derivative is selected from the group consisting of:
(a) curcumin;
(b) a curcuminoid of formula (I) in which:
(i) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;
(ii) R₁ is —H; R₂ is —OH; R₃ is —H or —OH; R₄ is —H, R₅ is —H; R₆ is —OH; and R₇ is —H or —OH;
(iii) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;
(iv) R₁ is —OH, R₂ is —OH, R₃ is —OH, R₄ is —H or —OH, R₅ is —OH, R₆ is —OH; and R₇ is —OH;
(v) R₁ is —OCH₃; R₂ is —OCH₃; R₃ is —OCH₃; R₄ is —H or —OH; R₅ is —OCH₃; R₆ is —OCH₃; and R₇ is —OCH₃;
(vi) R₁ is —H; R₂ is —OCH₃; R₃ is —OCH₃; R₄ is —H or —OH; R₅ is —H; R₆ is —OCH₃; and R₇ is —OCH₃;
(vii) R₁ is —H; R₂ is —OH; R₃ is —H; R₄ is —H; R₅ is —H or —OH; R₆ is —OH; and R₇ is —H;
(viii) R₁ is —H; R₂ is —OCH₃; R₃ is —H; R₄ is —H; R₅ is —H or —OH; R₆ is —OCH₃; and R₇ is —H; or
(ix) R₁ is —OH; R₂ is —OCH₃; R₃ is —H or —OH; R₄ is H or —OH; R₅ is —OH; R₆ is —OCH₃; and R₇ is —H or —OH;
(c) a curcuminoid of formula (II) in which the alternatives for R₁ through R₇ are the same as those recited in paragraph (b);
(d) a curcuminoid of formula (III) in which the alternatives for R₁ through R₇ are the same as those recited in paragraph (b);
(e) the compound of formula (IV) in which X is —H, the compound being designated furfural curcuminoid;
(f) an analogue of furfural curcuminoid in which X is —OH, ethyl, methyl, or acetyl;
(g) the compound of formula (V) in which X is —H, the compound being designated salicyl curcuminoid;
(h) an analogue of salicyl curcuminoid in which X is —OH, ethyl, methyl, or acetyl;
(i) the compound of formula (VI) in which X is —H, the compound being designated veratryl curcuminoid;
(j) an analogue of veratryl curcuminoid in which X is —OH, ethyl, methyl, or acetyl;
(k) the compound of formula (VII) in which X is —H, the compound being designated p-anisyl curcuminoid;
(l) an analogue of p-anisyl curcuminoid in which X is —OH, ethyl, methyl, or acetyl;
(m) the compound of formula (VIII) in which X is —H, the compound being designated piperonal curcuminoid;
(n) an analogue of piperonal curcuminoid in which X is —OH, ethyl, methyl, or acetyl;
(o) a tetrahydrocurcuminoid of formula (IX) in which the alternatives for R₁ through R₇ are the same as those recited in paragraph (b);
(p) a curcuminoid of formula (X) in which the alternatives for R₁ through R₇ are the same as those recited in paragraph (b);
(q) a curcuminoid of formula (XI) in which the alternatives for R₁ through R₇ are the same as those recited in paragraph (b);
(r) a reduced curcuminoid of formula (XII) in which the alternatives for R₁ through R₇ are the same as those recited in paragraph (b);
(s) derivatives of the compounds recited in (b) through (r) in which any of the methoxy groups are replaced with lower alkoxy groups selected from the group consisting of ethoxy, n-propoxy, and isopropoxy;
(t) derivatives of the compounds recited in (b) through (r) in which any of the hydroxy groups of the phenolic moieties are substituted with an acyl group selected from the group consisting of acetyl, propionyl, butyryl, and isobutyryl;
(u) analogues of the compounds recited in (b), (c), and (e) through (p) in which one or both of the carbonyl (CO) groups are replaced by amino (NH) groups in analogy with formulas II and III; and
(v) analogues of the compounds recited in (b), (c), and (e) through (p) in which one or both of the oxygens of the carbonyl groups are replaced by sulfur to form thiocarbonyl groups.

54. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin, a soluble curcuminoid, or a soluble curcumin derivative in a solution containing at least one alcohol to a mammal to detectably inhibit the activity of phosphorylase kinase in the blood of the mammal or in a tissue of the mammal, the condition or disease being selected from the group consisting of infections caused by bacteria, superficial fungi, deep fungi, viruses, mycoplasmas, and parasites.

55. The method of claim 54 wherein the curcumin, curcuminoid, or curcumin derivative is selected from the group consisting of:
(a) curcumin;
(b) a curcuminoid of formula (I) in which:
(i) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;
(ii) R₁ is —H; R₂ is —OH; R₃ is —H or —OH; R₄ is —H, R₅ is —H; R₆ is —OH; and R₇ is —H or —OH;
(iii) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;
(iv) R₁ is —OH, R₂ is —OH, R₃ is —OH, R₄ is —H or —OH, R₅ is —OH, R₆ is —OH; and R₇ is —OH;
(v) R₁ is —OCH₃; R₂ is —OCH₃; R₃ is —OCH₃; R₄ is —H or —OH; R₅ is —OCH₃; R₆ is —OCH₃; and R₇ is —OCH₃;
(vi) R₁ is —H; R₂ is —OCH₃; R₃ is —OCH₃; R₄ is —H or —OH; R₅ is —H; R₆ is —OCH₃; and R₇ is —OCH₃;
(vii) R₁ is —H; R₂ is —OH; R₃ is —H; R₄ is —H; R₅ is —H or —OH; R₆ is —OH; and R₇ is —H;
(viii) R₁ is —H; R₂ is —OCH₃; R₃ is —H; R₄ is —H; R₅ is —H or —OH; R₆ is —OCH₃; and R₇ is —H; or
(ix) R₁ is —OH; R₂ is —OCH₃; R₃ is —H or —OH; R₄ is H or —OH; R₅ is —OH; R₆ is —OCH₃; and R₇ is —H or —OH;
(c) a curcuminoid of formula (II) in which the alternatives for R₁ through R₇ are the same as those recited in paragraph (b);
(d) a curcuminoid of formula (III) in which the alternatives for R₁ through R₇ are the same as those recited in paragraph (b);
(e) the compound of formula (IV) in which X is —H, the compound being designated furfural curcuminoid;
(f) an analogue of furfural curcuminoid in which X is —OH, ethyl, methyl, or acetyl;
(g) the compound of formula (V) in which X is —H, the compound being designated salicyl curcuminoid;
(h) an analogue of salicyl curcuminoid in which X is —OH, ethyl, methyl, or acetyl;
(i) the compound of formula (VI) in which X is —H, the compound being designated veratryl curcuminoid;
(j) an analogue of veratryl curcuminoid in which X is —OH, ethyl, methyl, or acetyl;
(k) the compound of formula (VII) in which X is —H, the compound being designated p-anisyl curcuminoid;
(l) an analogue of p-anisyl curcuminoid in which X is —OH, ethyl, methyl, or acetyl;
(m) the compound of formula (VIII) in which X is —H, the compound being designated piperonal curcuminoid;
(n) an analogue of piperonal curcuminoid in which X is —OH, ethyl, methyl, or acetyl;
(o) a tetrahydrocurcuminoid of formula (IX) in which the alternatives for R₁ through R₇ are the same as those recited in paragraph (b);
(p) a curcuminoid of formula (X) in which the alternatives for R₁ through R₇ are the same as those recited in paragraph (b);
(q) a curcuminoid of formula (XI) in which the alternatives for R₁ through R₇ are the same as those recited in paragraph (b);
(r) a reduced curcuminoid of formula (XII) in which the alternatives for R₁ through R₇ are the same as those recited in paragraph (b);
(s) derivatives of the compounds recited in (b) through (r) in which any of the methoxy groups are replaced with lower alkoxy groups selected from the group consisting of ethoxy, n-propoxy, and isopropoxy;
(t) derivatives of the compounds recited in (b) through (r) in which any of the hydroxy groups of the phenolic moieties are substituted with an acyl group selected from the group consisting of acetyl, propionyl, butyryl, and isobutyryl;
(u) analogues of the compounds recited in (b), (c), and (e) through (p) in which one or both of the carbonyl (CO) groups are replaced by amino (NH) groups in analogy with formulas II and III; and
(v) analogues of the compounds recited in (b), (c), and (e) through (p) in which one or both of the oxygens of the carbonyl groups are replaced by sulfur to form thiocarbonyl groups.

56. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin, a soluble curcuminoid, or a soluble curcumin derivative in a solution containing at least one alcohol to a mammal to detectably inhibit the activity of phosphorylase kinase in the blood of the mammal or in a tissue of the mammal, the condition or disease being diabetes.

57. The method of claim 56 wherein the curcumin, curcuminoid, or curcumin derivative is selected from the group consisting of:
(a) curcumin;
(b) a curcuminoid of formula (I) in which:
(i) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;
(ii) R₁ is —H; R₂ is —OH; R₃ is —H or —OH; R₄ is —H, R₅ is —H; R₆ is —OH; and R₇ is —H or —OH;
(iii) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;
(iv) R₁ is —OH, R₂ is —OH, R₃ is —OH, R₄ is —H or —OH, R₅ is —OH, R₆ is —OH; and R₇ is —OH;
(v) R₁ is —OCH₃; R₂ is —OCH₃; R₃ is —OCH₃; R₄ is —H or —OH; R₅ is —OCH₃; R₆ is —OCH₃; and R₇ is —OCH₃;
(vi) R₁ is —H; R₂ is —OCH₃; R₃ is —OCH₃; R₄ is —H or —OH; R₅ is —H; R₆ is —OCH₃; and R₇ is —OCH₃;
(vii) R₁ is —H; R₂ is —OH; R₃ is —H; R₄ is —H; R₅ is —H or —OH; R₆ is —OH; and R₇ is —H;
(viii) R₁ is —H; R₂ is —OCH₃; R₃ is —H; R₄ is —H; R₅ is —H or —OH; R₆ is —OCH₃; and R₇ is —H; or
(ix) R₁ is —OH; R₂ is —OCH₃; R₃ is —H or —OH; R₄ is H or —OH; R₅ is —OH; R₆ is —OCH₃; and R₇ is —H or —OH;
(c) a curcuminoid of formula (II) in which the alternatives for R₁ through R₇ are the same as those recited in paragraph (b);
(d) a curcuminoid of formula (III) in which the alternatives for R₁ through R₇ are the same as those recited in paragraph (b);
(e) the compound of formula (IV) in which X is —H, the compound being designated furfural curcuminoid;
(f) an analogue of furfural curcuminoid in which X is —OH, ethyl, methyl, or acetyl;
(g) the compound of formula (V) in which X is —H, the compound being designated salicyl curcuminoid;
(h) an analogue of salicyl curcuminoid in which X is —OH, ethyl, methyl, or acetyl;
(i) the compound of formula (VI) in which X is —H, the compound being designated veratryl curcuminoid;
(J) an analogue of veratryl curcuminoid in which X is —OH, ethyl, methyl, or acetyl;
(k) the compound of formula (VII) in which X is —H, the compound being designated p-anisyl curcuminoid;
(l) an analogue of p-anisyl curcuminoid in which X is —OH, ethyl, methyl, or acetyl;
(m) the compound of formula (VIII) in which X is —H, the compound being designated piperonal curcuminoid;
(n) an analogue of piperonal curcuminoid in which X is —OH, ethyl, methyl, or acetyl;
(o) a tetrahydrocurcuminoid of formula (IX) in which the alternatives for R₁ through R₇ are the same as those recited in paragraph (b);
(p) a curcuminoid of formula (X) in which the alternatives for R₁ through R₇ are the same as those recited in paragraph (b);
(q) a curcuminoid of formula (XI) in which the alternatives for R₁ through R₇ are the same as those recited in paragraph (b);
(r) a reduced curcuminoid of formula (XII) in which the alternatives for R₁ through R₇ are the same as those recited in paragraph (b);
(s) derivatives of the compounds recited in (b) through (r) in which any of the methoxy groups are replaced with lower alkoxy groups selected from the group consisting of ethoxy, n-propoxy, and isopropoxy;
(t) derivatives of the compounds recited in (b) through (r) in which any of the hydroxy groups of the phenolic moieties are substituted with an acyl group selected from the group consisting of acetyl, propionyl, butyryl, and isobutyryl;
(u) analogues of the compounds recited in (b), (c), and (e) through (p) in which one or both of the carbonyl (CO) groups are replaced by amino (NH) groups in analogy with formulas II and III; and
(v) analogues of the compounds recited in (b), (c), and (e) through (p) in which one or both of the oxygens of the carbonyl groups are replaced by sulfur to form thiocarbonyl groups.

58. A method for treating a condition or disease in a mammal by inhibiting the breakdown of glycogen and the generation of ATP through phosphorylase kinase inhibition in order to inhibit the energy supply for at least one cellular activity selected from the group consisting of cell migration, cell proliferation, cytokine secretion, growth factor secretion and gene transcription, the method comprising administering soluble curcumin, a soluble curcuminoid, or a soluble curcumin derivative in a solution containing at least one alcohol to a mammal to detectably inhibit the activity of phosphorylase kinase in the blood of the mammal or in a tissue of the mammal, the condition or disease being a neurodegenerative condition.

59. The method of claim 58 wherein the curcumin, curcuminoid, or curcumin derivative is selected from the group consisting of:
(a) curcumin;
(b) a curcuminoid of formula (I) in which:
(i) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;
(ii) R₁ is —H; R₂ is —OH; R₃ is —H or —OH; R₄ is —H, R₅ is —H; R₆ is —OH; and R₇ is —H or —OH;
(iii) each of R₁, R₂, and R₃ is —H, —OCH₃, —OH, —ONa, acetyl, methyl, or ethyl; R₄ is —H, —OH, ethyl, methyl, or acetyl; and each of R₅, R₆, and R₇ is —H, —OCH₃, —OH, —ONa, acetyl, methyl, or ethyl, wherein if R₄ is —H or —OH, at least one of R₂ and R₆ is other than —H or —OH;
(iv) R₁ is —OH, R₂ is —OH, R₃ is —OH, R₄ is —H or —OH, R₅ is —OH, R₆ is —OH; and R₇ is —OH;
(v) R₁ is —OCH₃; R₂ is —OCH₃; R₃ is —OCH₃; R₄ is —H or —OH; R₅ is —OCH₃; R₆ is —OCH₃; and R₇ is —OCH₃;
(vi) R₁ is —H; R₂ is —OCH₃; R₃ is —OCH₃; R₄ is —H or —OH; R₅ is —H; R₆ is —OCH₃; and R₇ is —OCH₃;
(vii) R₁ is —H; R₂ is —OH; R₃ is —H; R₄ is —H; R₅ is —H or —OH; R₆ is —OH; and R₇ is —H;
(viii) R₁ is —H; R₂ is —OCH₃; R₃ is —H; R₄ is —H; R₅ is —H or —OH; R₆ is —OCH₃; and R₇ is —H; or
(ix) R₁ is —OH; R₂ is —OCH₃; R₃ is —H or —OH; R₄ is H or —OH; R₅ is —OH; R₆ is —OCH₃; and R₇ is —H or —OH;
(c) a curcuminoid of formula (II) in which the alternatives for R₁ through R₇ are the same as those recited in paragraph (b);
(d) a curcuminoid of formula (III) in which the alternatives for R₁ through R₇ are the same as those recited in paragraph (b);
(e) the compound of formula (IV) in which X is —H, the compound being designated furfural curcuminoid;
(f) an analogue of furfural curcuminoid in which X is —OH, ethyl, methyl, or acetyl;
(g) the compound of formula (V) in which X is —H, the compound being designated salicyl curcuminoid;
(h) an analogue of salicyl curcuminoid in which X is —OH, ethyl, methyl, or acetyl;
(i) the compound of formula (VI) in which X is —H, the compound being designated veratryl curcuminoid;
(j) an analogue of veratryl curcuminoid in which X is —OH, ethyl, methyl, or acetyl;
(k) the compound of formula (VII) in which X is —H, the compound being designated p-anisyl curcuminoid;
(l) an analogue of p-anisyl curcuminoid in which X is —OH, ethyl, methyl, or acetyl;
(m) the compound of formula (VIII) in which X is —H, the compound being designated piperonal curcuminoid;
(n) an analogue of piperonal curcuminoid in which X is —OH, ethyl, methyl, or acetyl;
(o) a tetrahydrocurcuminoid of formula (IX) in which the alternatives for R₁ through R₇ are the same as those recited in paragraph (b);
(p) a curcuminoid of formula (X) in which the alternatives for R₁ through R₇ are the same as those recited in paragraph (b);
(q) a curcuminoid of formula (XI) in which the alternatives for R₁ through R₇ are the same as those recited in paragraph (b);
(r) a reduced curcuminoid of formula (XII) in which the alternatives for R₁ through R₇ are the same as those recited in paragraph (b);
(s) derivatives of the compounds recited in (b) through (r) in which any of the methoxy groups are replaced with lower alkoxy groups selected from the group consisting of ethoxy, n-propoxy, and isopropoxy;
(t) derivatives of the compounds recited in (b) through (r) in which any of the hydroxy groups of the phenolic moieties are substituted with an acyl group selected from the group consisting of acetyl, propionyl, butyryl, and isobutyryl;
(u) analogues of the compounds recited in (b), (c), and (e) through (p) in which one or both of the carbonyl (CO) groups are replaced by amino (NH) groups in analogy with formulas II and III; and
(v) analogues of the compounds recited in (b), (c), and (e) through (p) in which one or both of the oxygens of the carbonyl groups are replaced by sulfur to form thiocarbonyl groups.

60. The method of claim 1 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone.

61. The method of claim 60 wherein the additional compound is selected from the group consisting of vitamin D₃ and a vitamin D₃ analogue selected from the group consisting of calcitriol, calcipotriene, calcipotriol, and tacalcitol.

62. The method of claim 60 wherein the additional compound is selected from the group consisting of vitamin A, a vitamin A derivative, and a vitamin A analogue.

63. The method of claim 60 wherein the additional compound is a calmodulin inhibitor selected from the group consisting of zinc, cyclosporin A, anthralin, and trifluoroperazine.

64. The method of claim 60 wherein the additional compound is an anti-inflammatory drug selected from the group consisting of a corticosteroid, a substance P inhibitor, a capsaicin-sensitive vanilloid receptor inhibitor, a cyclo-oxygenase inhibitor, and another non-steroidal anti-inflammatory agent.

65. The method of claim 60 wherein the additional compound is a calcium channel blocker selected from the group consisting of diltiazem, nifedepine, isradipine, and verapamil.

66. The method of claim 60 wherein the additional compound is a H1 histamine blocker or a H2 histamine blocker, wherein the H1 histamine blocker is selected from the group consisting of carbinoxamine maleate, clemastine fumarate, diphenhydramine hydrochloride, dimenhydrinate, pyrilamine maleate, tripelennamine hydrochloride, tripelennamine citrate, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, acrivastine, cetirizine hydrochloride, astemizole, levocabastine hydrochloride, loratadine, and terfenadine, and wherein the H2 histamine blocker is selected from the group consisting of cimetidine, ranitidine, famotidine, and nizatidine.

67. The method of claim 60 wherein the additional compound is an antioxidant or free radical scavenger selected from the group consisting of α-tocopherol, β-carotene, reduced glutathione, catalase, and superoxide dismutase.

68. The method of claim 60 wherein the additional compound is a polyphenolic compound selected from the group consisting of (−)epigallocatechin-3-gallate, epigallocatechin, rutin, catechin, epicatechin, naringin, naringenin, and gallotanin.

69. The method of claim 60 wherein the additional compound is a monoterpene selected from the group consisting of d-limonene and perillyl alcohol.

70. The method of claim 60 wherein the additional compound is genistein.

71. The method of claim 60 wherein the additional compound is the soybean derived lectin soybean agglutinin.

72. The method of claim 60 wherein the additional compound is dehydrozingerone.

73. The method of claim 15 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and.
(l) dehydrozingerone.

74. The method of claim 73 wherein the additional compound is selected from the group consisting of vitamin D₃ and a vitamin D₃ analogue selected from the group consisting of calcitriol, calcipotriene, calcipotriol, and tacalcitol.

75. The method of claim 73 wherein the additional compound is selected from the group consisting of vitamin A, a vitamin A derivative, and a vitamin A analogue.

76. The method of claim 73 wherein the additional compound is a calmodulin inhibitor selected from the group consisting of zinc, cyclosporin A, anthralin, and trifluoroperazine.

77. The method of claim 73 wherein the additional compound is an anti-inflammatory drug selected from the group consisting of a corticosteroid, a substance P inhibitor, a capsaicin-sensitive vanilloid receptor inhibitor, a cyclo-oxygenase inhibitor, and another non-steroidal anti-inflammatory agent.

78. The method of claim 73 wherein the additional compound is a calcium channel blocker selected from the group consisting of diltiazem, nifedepine, isradipine, and verapamil.

79. The method of claim 73 wherein the additional compound is a H1 histamine blocker or a H2 histamine blocker, wherein the H1 histamine blocker is selected from the group consisting of carbinoxamine maleate, clemastine fumarate, diphenhydramine hydrochloride, dimenhydrinate, pyrilamine maleate, tripelennamine hydrochloride, tripelennamine citrate, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, acrivastine, cetirizine hydrochloride, astemizole, levocabastine hydrochloride, loratadine, and terfenadine, and wherein the H2 histamine blocker is selected from the group consisting of cimetidine, ranitidine, famotidine, and nizatidine.

80. The method of claim 73 wherein the additional compound is an antioxidant or free radical scavenger selected from the group consisting of α-tocopherol, β-carotene, reduced glutathione, catalase, and superoxide dismutase.

81. The method of claim 73 wherein the additional compound is a polyphenolic compound selected from the group consisting of (−)epigallocatechin-3-gallate, rutin, catechin, epicatechin, naringin, naringenin, epigallocatechin, and gallotanin.

82. The method of claim 73 wherein the additional compound is a monoterpene selected from the group consisting of d-limonene and perillyl alcohol.

83. The method of claim 73 wherein the additional compound is genistein.

84. The method of claim 73 wherein the additional compound is the soybean derived lectin soybean agglutinin.

85. The method of claim 73 wherein the additional compound is dehydrozingerone.

86. The method of claim 30 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone.

87. The method of claim 33 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone.

88. The method of claim 36 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone.

89. The method of claim 39 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone.

90. The method of claim 42 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(1) dehydrozingerone

91. The method of claim 45 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone

92. The method of claim 48 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and.
(l) dehydrozingerone

93. The method of claim 50 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone

94. The method of claim 52 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone.

95. The method of claim 54 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone.

96. The method of claim 56 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone

97. The method of claim 58 further comprising administering to the mammal at least one additional compound, the additional compound being selected from the group consisting of:
(a) vitamin D₃ and vitamin D₃ analogues;
(b) vitamin A, vitamin A derivatives, and vitamin A analogues
(c) a calmodulin inhibitor;
(d) an anti-inflammatory drug;
(e) a calcium channel blocker;
(f) a H1 or H2 histamine blocker;
(g) an antioxidant or free radical scavenger;
(h) a polyphenolic compound;
(i) a monoterpene;
(j) genistein;
(k) a soybean derived lectin; and
(l) dehydrozingerone

98. A pharmaceutical composition comprising:
(a) curcumin, a curcuminoid, or a curcumin derivative in a solution containing at least one alcohol, the curcumin, curcuminoid, or curcumin derivative being present in a quantity sufficient to detectably inhibit the activity of phosphorylase kinase in the blood of the mammal or in a tissue of the mammal to which the composition is administered;
(b) at least one additional compound, the additional compound being selected from the group consisting of:
(1) vitamin D₃ and vitamin D₃ analogues;
(2) vitamin A, vitamin A derivatives, and vitamin A analogues
(3) a calmodulin inhibitor;
(4) an anti-inflammatory drug;
(5) a calcium channel blocker;
(6) a H1 or H2 histamine blocker;
(7) an antioxidant or free radical scavenger;
(8) a polyphenolic compound;
(9) a monoterpene;
(10) genistein;
(11) a soybean derived lectin; and
(12) dehydrozingerone; and
(c) a pharmaceutically acceptable carrier.

99. The pharmaceutical composition of claim 98 wherein the curcumin, curcuminoid, or curcumin derivative is selected from the group consisting of:
(a) curcumin;
(b) a curcuminoid of formula (I) in which:
(i) R₁ is —H or —OCH₃; R₂ is —OH; R₃ is —H; R₄ is H; R₅ is —H or OCH₃; R₆ is —OH, and R₇ is —H, wherein only one of R₁ and R₅ is —OCH₃;
(ii) R₁ is —H; R₂ is —OH; R₃ is —H or —OH; R₄ is —H, R₅ is —H; R₆ is —OH; and R₇ is —H or —OH;
(iii) R₁ is —OCH₃; R₂ is —OH, —ONa, acetyl, methyl, or ethyl; R₃ is —H; R₄ is —H, —OH, ethyl, methyl, or acetyl; R₅ is —OCH₃; R₆ is —OH, —ONa, acetyl, methyl, or ethyl; and R₇ is —H; wherein, if R₄ is —H, at least one of R₂ and R₆ is other than —OH;
(iv) R₁ is —OH, R₂ is —OH, R₃ is —OH, R₄ is —H, R₅ is —OH, R₆ is —OH; and R₇ is —OH;
(v) R₁ is —OCH₃; R₂ is —OCH₃; R₃ is —OCH₃; R₄ is —H; R₅ is —OCH₃; R₆ is —OCH₃; and R₇ is —OCH₃;
(vi) R₁ is —H; R₂ is —OCH₃; R₃ is —OCH₃; R₄ is —H; R₅ is —H; R₆ is —OCH₃; and R₇ is —OCH₃;
(vii) R₁ is —H; R₂ is —OH; R₃ is —H; R₄ is —H; R₅ is —H; R₆ is —OH; and R₇ is —H;
(viii) R₁ is —H; R₂ is —OCH₃; R₃ is —H; R₄ is —H; R₅ is —H; R₆ is OCH₃; and R₇ is —H; or
(ix) R₁ is —OH; R₂ is —OCH₃; R₃ is —H or —OH; R₄ is H; R₅ is —OH; R₆ is —OCH₃; and R₇ is —H or —OH;
(c) a curcuminoid of formula (II) in which the alternatives for R₁ through R₇ are the same as those recited in paragraph (b);
(d) a curcuminoid of formula (III) in which the alternatives for R₁ through R₇ are the same as those recited in paragraph (b);
(e) the compound of formula (IV) in which X is —H, the compound being designated furfural curcuminoid;
(f) an analogue of furfural curcuminoid in which X is —OH, ethyl, methyl, or acetyl;
(g) the compound of formula (V) in which X is —H, the compound being designated salicyl curcuminoid;
(h) an analogue of salicyl curcuminoid in which X is —OH, ethyl, methyl, or acetyl;
(i) the compound of formula (VI) in which X is —H, the compound being designated veratryl curcuminoid;
(j) an analogue of veratryl curcuminoid in which X is —OH, ethyl, methyl, or acetyl;
(k) the compound of formula (VII) in which X is —H, the compound being designated p-anisyl curcuminoid;
(l) an analogue of p-anisyl curcuminoid in which X is —OH, ethyl, methyl, or acetyl;
(m) the compound of formula (VIII) in which X is —H, the compound being designated piperonal curcuminoid;
(n) an analogue of piperonal curcuminoid in which X is —OH, ethyl, methyl, or acetyl;
(o) a tetrahydrocurcuminoid of formula (IX) in which the alternatives for R₁ through R₇ are the same as those recited in paragraph (b);
(p) a curcuminoid of formula (X) in which the alternatives for R₁ through R₇ are the same as those recited in paragraph (b);
(q) a curcuminoid of formula (XI) in which the alternatives for R₁ through R₇ are the same as those recited in paragraph (b);
(r) a reduced curcuminoid of formula (XII) in which the alternatives for R₁ through R₇ are the same as those recited in paragraph (b);
(s) derivatives of the compounds recited in (b) through (r) in which either or both of the methoxy groups are replaced with lower alkoxy groups selected from the group consisting of ethoxy, n-propoxy, and isopropoxy;
(t) derivatives of the compounds recited in (b) through (r) in which either or both of the hydroxy groups of the phenolic moieties are substituted with an acyl group selected from the group consisting of acetyl, propionyl, butyryl, and isobutyryl;
(u) analogues of the compounds recited in (d) through (r) in which one or both of the carbonyl (CO) groups are replaced by amino (NH) groups in analogy with formulas II and III; and
(v) analogues of the compounds recited in (b), (c), and (d) through (r) in which one or both of the oxygens of the carbonyl groups are replaced by sulfur to form thiocarbonyl groups.

100. The pharmaceutical composition of claim 98 wherein the additional compound is selected from the group consisting of vitamin D₃ and a vitamin D₃ analogue selected from the group consisting of calcitriol, calcipotriene, calcipotriol, and tacalcitol.

101. The pharmaceutical composition of claim 98 wherein the additional compound is selected from the group consisting of vitamin A, a vitamin A derivative, and a vitamin A analogue.

102. The pharmaceutical composition of claim 98 wherein the additional compound is a calmodulin inhibitor selected from the group consisting of zinc, cyclosporin A, anthralin, and trifluoroperazine.

103. The pharmaceutical composition of claim 98 wherein the additional compound is an anti-inflammatory drug selected from the group consisting of a corticosteroid, a substance P inhibitor, a capsaicin-sensitive vanilloid receptor inhibitor, a cyclo-oxygenase inhibitor, and another non-steroidal anti-inflammatory agent.

104. The pharmaceutical composition of claim 98 wherein the additional compound is a calcium channel blocker selected from the group consisting of diltiazem, nifedepine, isradipine, and verapamil.

105. The pharmaceutical composition of claim 98 wherein the additional compound is a H1 histamine blocker or a H2 histamine blocker, wherein the H1 histamine blocker is selected from the group consisting of carbinoxamine maleate, clemastine fumarate, diphenhydramine hydrochloride, dimenhydrinate, pyrilamine maleate, tripelennamine hydrochloride, tripelennamine citrate, chlorpheniramine maleate, brompheniramine maleate, hydroxyzine hydrochloride, hydroxyzine pamoate, cyclizine hydrochloride, cyclizine lactate, meclizine hydrochloride, promethazine hydrochloride, acrivastine, cetirizine hydrochloride, astemizole, levocabastine hydrochloride, loratadine, and terfenadine, and wherein the H2 histamine blocker is selected from the group consisting of cimetidine, ranitidine, famotidine, and nizatidine.

106. The pharmaceutical composition of claim 98 wherein the additional compound is an antioxidant or free radical scavenger selected from the group consisting of α-tocopherol, β-carotene, reduced glutathione, catalase, and superoxide dismutase.

107. The pharmaceutical composition of claim 98 wherein the additional compound is a polyphenolic compound selected from the group consisting of (−)epigallocatechin-3-gallate, rutin, catechin, epicatechin, naringin, naringenin, epigallocatechin, and gallotanin.

108. The pharmaceutical composition of claim 98 wherein the additional compound is a monoterpene selected from the group consisting of d-limonene and perillyl alcohol.

109. The pharmaceutical composition of claim 98 wherein the additional compound is genistein.

110. The pharmaceutical composition of claim 98 wherein the additional compound is the soybean derived lectin soybean agglutinin.

111. The pharmaceutical composition of claim 98 wherein the additional compound is dehydrozingerone.

112. The pharmaceutical composition of claim 98 wherein the curcumin, curcumin derivative, or curcuminoid is present as a boron complex.

113. The pharmaceutical composition of claim 112 wherein the boron complex is selected from the group consisting of
(a) a difluoroboron complex;
(b) a mixed complex in which the two fluorine atoms of a difluoroboron complex are replaced with the carboxyl oxygens of oxalic acid;
(c) a mixed complex in which the two fluorine atoms of a difluoroboron complex are replaced with a carboxyl group and a hydroxyl group of citric acid;
(d) a mixed complex in which the two fluorine atoms of a difluoroboron complex are replaced with the two hydroxyl groups of dibenzyl tartramide; and
(e) a mixed complex in which the two fluorine atoms of a difluoroboron complex are replaced with a second molecule of curcumin, the curcumin derivative, or the curcuminoid.

114. The pharmaceutical composition of claim 98 wherein the curcumin, curcumin derivative, or curcuminoid is present in a liposome.

115. The pharmaceutical composition of claim 114 wherein the pharmaceutical composition is a preparation selected from the group consisting of a skin preparation, an eye drop preparation, a nasal drop preparation, an oral preparation, a pharyngeal preparation, a rectal preparation, a vaginal preparation, a bladder preparation, a urethral preparation, a parenteral preparation, and a bronchial preparation.
